(19) United States
(12) Patent Application Publication
(10) Pub. No.: US 2007/0212677 A1
(43) Pub. Date: Sep. 13, 2007

(54) Identifying off-target effects and hidden phenotypes of drugs in human cells

(75) Inventors: Marnie L. MacDonald, Pleasanton, CA (US); Jane Lamerdin, Livermore, CA (US); Stephen Owens, Martinez, CA (US); Brigitte Keon, Castro Valley, CA (US); Graham K. Bilter, Pleasanton, CA (US); Zhidi Shang, Fremont, CA (US); Zhengping Huang, Pleasanton, CA (US); Helen Yu, Mountain View, CA (US); Jennifer Dias, Dublin, CA (US); Tomoe Minami, Dublin, CA (US); Stephen W. Michnick, Montreal (CA); John K. Westwick, San Ramon, CA (US)

Correspondence Address: Isaac A. Angres, Suite 301, 2001 Jefferson Davis Highway, Arlington, VA, 22202, US

(73) Assignee: Odyssey Thera, Inc., San Ramon, CA (US)

(21) Appl. No.: 11/513,068

(22) Filed: Aug. 31, 2006

Related U.S. Application Data
(63) Continuation-in-part of application No. 11/282,745, filed on Nov. 21, 2005.
(60) Provisional application No. 60/712,812, filed on Sep. 1, 2005.
(60) Provisional application No. 60/629,558, filed on Nov. 22, 2004.

(51) Int. Cl. C12Q 1/00; C12Q 1/68; G01N 33/53; G06F 19/00

(52) U.S. Cl. 435/4; 435/6; 435/7.1; 702/19

(57) ABSTRACT

This invention provides principles, methods and compositions for ascertaining the mechanism of action of pharmacologically important compounds in the context of network biology, across the entire scope of the complex pathways of living cells. Importantly, the principles, methods and compositions provided allow a rapid assessment of the on-pathway and off-pathway effects of lead compounds and drug candidates in living cells, and comparisons of lead compounds with well-characterized drugs and toxicants to identify patterns associated with efficacy and toxicity. The invention will be useful in improving the drug discovery process, in particular by identifying drug leads with desired safety and efficacy and in effecting early attrition of compounds with potential adverse effects in man.

This application claims the priority benefit under 35 U.S.C. section 119 of U.S. Provisional Patent Application No. 60/712,812 entitled “Identifying Off-Target Effects And Hidden Phenotypes Of Drugs In Human Cells” filed Sep. 1, 2005, which is in its entirety herein incorporated by reference. This application is also a continuation-in-part application of U.S. Ser. No. 11/282,745 filed Nov. 21, 2005, which application claims priority from U.S. Provisional Patent Application No. 60/629,558 filed Nov. 22, 2004.

### BACKGROUND OF THE INVENTION

The central challenge of the pharmaceutical industry is to develop drugs that are both safe and effective in man. Even an exquisitely selective chemical compound that binds to a therapeutic target may have completely unexpected or ‘off-pathway’ effects in living cells, leading to expensive pre-clinical and clinical failures. Regardless of whether a drug or drug candidate is an agonist, antagonist, inhibitor or activator of a target, drugs exert their actions by binding to a target protein and altering the function of that protein. For the purposes of this invention, we define ‘off pathway’ activity as any activity of a compound on a cellular target or pathway other than the intended target of the compound.

As evidenced by the 75% failure rate of drugs in clinical trials, the development of new drugs is a costly and unpredictable process, despite the number of research tools available to the pharmaceutical industry. The US Food and Drug Administration has estimated that even a 10% improvement in identifying adverse effects of compounds, prior to clinical trials, could save $100 Million in development costs per drug (reference white paper). Our central premise is that the off-pathway effects of new drugs are responsible for many if not all of the failures in new drug development. An understanding of the full spectrum of biological activity of any new chemical entity would help to identify potentially adverse effects of drugs prior to clinical trials. Therefore, we sought to establish a rapid method to assess the activity of any new chemical entity in the context of the complex networks of living cells.

Numerous in vivo and in vitro approaches are aimed at assessing the selectivity of lead compounds. Typical methods are briefly described here.

The selectivity of a compound can be assessed by constructing panels of in vitro assays to measure the activity of the compound against individual proteins in the target class. An example is the target class comprised of protein (tyrosine and serine/threonine) kinases. There are over 500 distinct protein kinases in the mammalian genome, making the development of selective inhibitors particularly challenging. A variety of companies (e.g. PanLabs, Kinexus) have established kinase inhibitor profiling products and services designed to assess the selectivity of lead compounds by testing each compound in vitro against panels of individual, purified kinases. The completion of the mapping of the ‘kinome’ and the availability of full-length genes encoding human kinases has aided in the development of such assay panels.

Although such assay panels exist for kinases, as well as for many other common drug target classes such as G-protein-coupled receptors (GPCRs), such panels are only capable of assessing drug activity against the proteins that are directly assayed. Even if it were possible to construct an assay for every kinase in the kinome, the approach would be limited in its ability to identify off-pathway effects of kinase leads. The most significant limitation is that even a highly selective inhibitor of a kinase may be capable of binding, activating, or inhibiting a plethora of other proteins that are not even in the same target class. Such off-target/off-pathway activities are unpredictable, and cannot be assessed in a comprehensive way with in vitro assays. Since the number of proteins in the proteome exceeds 30,000, a comprehensive analysis would require testing every compound of interest against over 30,000 proteins. First, it would be necessary to purify each of the thousands of proteins in the human proteome; and then to construct a biochemical assay to measure the activity of that particular protein; and finally, to assay each chemical compound of interest in 30,000 discrete assays. This is not practical or even feasible in the near future.

Other profiling approaches involve panning biological extracts or lysates for proteins that are capable of binding to a compound of interest. Such approaches typically involve contacting a cell lysate or tissue extract with a test compound that is bound to a bead or other solid surface and then analyzing the proteins bound to the bead. The proteins bound to the bead can be analyzed by mass spectroscopy, immunoprecipitation, or flow cytometry. Unfortunately, such methods often require concentrations of compound that are far higher than the physiological levels of any drug. In addition, artifacts can occur as a result of removing the proteins from their cellular milieu and subcellular context. For example, when a cell is lysed, proteins are released from their normal subcellular compartments and a particular protein may be capable of binding to a compound on a bead; whereas, in the living cell, the drug would never ‘see’ that protein.

To date, in vivo approaches to pharmacological profiling involve treating living cells, tissues or whole organisms with a test compound; then measuring changes in one or more phenomenological, functional or gene expression patterns of the cells, tissues or organisms in response to the test compound. Each of these is discussed below.

Phenomenological and functional assays allow an assessment of the spectrum of functional consequences of drug activity in living cells and a comparison of those responses to that of agents with known characteristics. For example, Dunnington et al. described methods for studying the function patterns of pharmacologically important compounds by measuring the effects of compounds on a plethora of physiological measurements in a variety of cell types (US 20030100997). They specified assays for cellular membrane potential, gene expression, physiological transport, cell proliferation, secretion, apoptosis, toxicity, light scattering, morphology, chemotaxis, adhesion, and similar parameters which represent measurable parameters of cell behavior or response. Importantly, the majority of these parameters are not molecular parameters per se. The phenomenological approach has the advantage of determining a broad scope of desired and undesired functional properties of compounds. However, it has the disadvantage of being purely descriptive, not allowing the determination of the biochemical mechanism of action of any undesired properties or of identifying properties that may not have immediate functional consequences. Also, unlike molecular parameters which number in the tens of thousands, there is a limited number and variety of phenomenological parameters that can be measured in any particular cell.

The use of gene microarrays for pharmacological profiling has become routine in the pharmaceutical industry. For this purpose, cells—or even whole animals—are treated with the drug or compound of interest. Following a period of time (usually 24-48 hours) messenger RNA is isolated from the cell or tissue. The pattern of expression of thousands of individual mRNAs in the absence and presence of the drug are compared. Microarrays have been used to predict which compounds have the greatest risk of side effects, and also to identify specific kinds of therapeutic activity. Gunther et al. (Proc Natl Acad Sci USA 100: 9608-9613) first produced a series of gene expression profiles by treating cells with chemicals known to act on the CNS and found that a small number of genes were good markers of antipsychotic, antidepressant, or opiate effects. U.S. Pat. No. 6,372,431 describes the use of gene microarrays to test samples treated with drug candidates in order to elucidate the gene expression pattern associated with drug treatment. This gene pattern can be compared with gene expression patterns associated with compounds which produce known metabolic and toxicological responses. Similarly, U.S. Pat. No. 5,569,588 discloses methods for drug screening by providing a plurality of separately isolated cells, each having an expression system with a different transcriptional regulatory element. Contacting this plurality of cells with a drug candidate and detecting reporter gene product signals from each cell provides a profile of response to the drug with regard to this multiplicity of regulatory elements.

In sum, high-throughput gene expression measurements using DNA microarrays provide global snapshots of the dynamics of gerie networks at the RNA level. However, transcription experiments only reveal the ultimate consequences of pathway perturbation, rather than the cause or mechanism. Gene network reconstruction from microarray data suffers from the so-called ‘dimensionality problem’ because the number of genes is much greater than the number of microarray experiments. Thus, simply identifying all of the mRNA species present and the levels at which they are present at a particular time, may not yield a complete picture of a particular drug. Moreover, changes in the level of individual mRNA molecules do not always correlate directly with the level or activity of the corresponding protein at a single point in time. Many proteins and other macromolecules undergo post-translational modifications and macromolecular interactions, which may affect the functions and activities of proteins within a tissue or cell independent of gene expression.

An alternative approach to pharmacological profiling is to analyze proteins that regulate the signaling pathways that, in turn, control gene expression and cell behavior. For example, cells or whole animals can be treated with the test compound, a cell lysate or tissue extract prepared, and the post-translational modification status of proteins assessed in the lysate or extract. The proteins in the cell lysates are typically either separated by 2-dimensional gel electrophoresis and then probed using Western blotting techniques, or are analyzed by multiplexed arrays of phospho-specific antibodies on beads or on antibody arrays (e.g. Nielsen et al., 2003, PNAS100: 9330-9335). Janes et al. (Molecular & Cellular Proteomics 2: 463-473, 2003) used this approach to develop a microtiter-plate-based assay panel for multiple kinase activities. Following treatment with agonists, cell lysates were assayed in microtiter wells precoated with anti-kinase antibodies and kinase activity measured with [32]P-ATP. These and similar biochemical methods provide information on what types of proteins are involved in a given pathway, their level of expression, and the way they interact with each other; but rarely can resolve where and when such proteins are activated within a cell. Traditional biochemical techniques are laborious, difficult to automate, and may require the use of radioactive reagents. Importantly, such techniques are not amenable to multiplexing with other types of assays, or to assaying thousands of drug candidates simultaneously.

Cells are complex systems in which a multitude of biochemical reactions and molecular events take place at one time and need to be finely orchestrated to preserve the cell homeostasis and direct the cell-specific functions. In particular, the flow of information from many different cellular inputs to a diverse set of physiological functions must rely on a precise organization of the intracellular signaling networks and on their timely and coordinated activation. In recent years, as a result of the development of new technologies based on real-time imaging of fluorescent indicators, the direct visualization of individual molecular events taking place in intact cells has become possible. The ability to work with living cells opens a new path to obtaining basic information critical to understanding the cell's molecular processes. These tools have been successfully applied to individual targets and high-throughput screening campaigns. The high spatial and temporal resolution that such methodologies provide opens the possibility to accurately measure quantitative and dynamic parameters of signaling networks in their complex cellular environment. However, with the exception of our own work and a single study of protein localization with respect to toxicology (see References) the prior art is silent on the application of network biology to pharmacological profiling of lead compounds or drug candidates. U.S. Pat. No. 6,673,554 provided protein localization assays for toxicity based on a single phenomenon, namely, intracellular translocation of proteins, in particular protein kinase C isozymes. While this reference embodies the use of whole cell assays it is limited to a single phenomenon that is restricted to a relatively small proportion of all the macromolecules of the cell.

The principles of network biology in living cells have never before been applied to drug discovery on a large scale. There are a number of factors that may have prevented its application until now. First, it is generally believed that it will take 20-30 years to solve the problem. In particular, ‘systems biology’ is perceived as a computational challenge which can only be solved when masses of descriptive information are in hand some years in the future. Second, current dogma holds that cell signaling events occur within seconds or even milliseconds, suggesting that dynamic events are difficult to capture except in rare circumstances and with the most sophisticated techniques. Third, biomolecular interactions that control pathways—such as protein-protein interactions—are generally perceived as events that can only be identified with static methods such as yeast two-hybrid screens. Fourth, the vast majority of small molecule drugs do not themselves disrupt protein-protein interactions; which means that attempts to study drug action by studying biomolecular complexes are often perceived as misguided attempts to perturb the interactions themselves. Finally, because of budget limitations, the majority of biochemical researchers studying drug action in cells do not utilize high throughput instrumentation to do so.

We sought to apply network biology to drug discovery in living cells on a large scale. The present invention provides a comprehensive rationale, principles, strategy, compositions and methodology for investigating the mechanism of action of any molecule in any living cell, including the identification of unintended or ‘off-pathway’ effects of the molecule of interest. The invention enables the creation of quantitative and predictive pharmacological profiles of lead compounds, drugs, and toxicants regardless of their intended mechanisms of action; and an assessment of unintended, off-pathway and/or adverse effects of molecules of pharmacological interest. The invention enables early attrition of lead compounds with undesirable properties, potentially saving millions of dollars spent on compounds with subsequent unintended or adverse effects in the clinical setting.

### OBJECTS AND ADVANTAGES OF THE INVENTION

It is an object of the present invention to provide principles for pharmacological profiling of chemical compounds, drug candidates, established drugs and toxicants on a global scale.

It is a further object of the invention to provide methods for assessing the activity, specificity, potency, time course, dose response and mechanism of action of chemical compounds in living cells.

It is also an object of the invention to allow determination of the selectivity of a chemical compound within the biological context of any cell.

It is an additional object of the present invention to allow detection of the potential off-pathway effects, adverse effects, or toxic effects of a chemical compound within the biological context of a particular cell type of interest.

It is an additional object of the invention to enable lead optimization, by performing pharmacological profiling of a collection or a series of lead compounds in an iterative manner until a desired pharmacological profile is obtained.

A further object of the invention is to enable attrition of drug candidates with undesirable or toxic properties.

It is a further object of the invention to establish pre-clinical safety profiles for new drug candidates.

It is a further object of the present invention to improve the efficiency of the drug discovery process by identifying unintended effects of lead compounds prior to clinical trials.

It is a further object of the present invention to improve the safety of first-in-class drugs by identifying adverse, toxic or other off-pathway effects prior to clinical trials.

It is an additional object of the present invention to identify positive or negative effects of drug excipients, carriers or drug delivery agents.

It is a further object of the present invention to provide methods suitable for the development of ‘designer drugs’ with predetermined properties.

An additional object of the invention is to enable the identification of new therapeutic indications for known drugs.

Another object of this invention is to provide a method for analyzing the activity of any class of pharmacological agent on any biochemical pathway.

A further object of this invention is to enable the identification of the biochemical pathways underlying drug toxicity.

A further object of this invention is to enable the identification of the biochemical pathways underlying drug efficacy for a broad range of diseases.

A further object of this invention is to provide methods, assays and compositions useful for drug discovery and development.

An additional object of the invention is to provide panels of assays suitable for pharmacological profiling.

The present invention has the advantage of being broadly applicable to any disease, pathway, gene, gene library, drug, drug target class, synthetic or natural product, chemical entity, assay format, detection mode, instrumentation, and cell type of interest.

### SUMMARY OF THE INVENTION

The present invention seeks to fulfill the above-mentioned needs for pharmaceutical discovery.

### BRIEF DESCRIPTION OF THE DRAWINGS

FIG. 1. Principle of the invention. Drugs, when administered to patients, enter the circulation and—if they have adequate pharmacokinetic properties—reach various organs, tissues and cells of the body. Drugs act upon the networks of living cells, which are the smallest living components of the human body. The scale-free networks that control cellular behavior are represented here as a circuit diagram within a cell. Cellular circuits are made up of molecules that form physical connections and undergo transitions in response to drugs and other extrinsic factors.

FIG. 2. Objective of the invention. Unknown effects of compounds of pharmacological interest can be either desirable (contributing to efficacy) or undesirable (contributing to toxicity). Pharmacologically active compounds increase or decrease the flux through pathways that are physically connected to the intended or unintended target of the compound. These effects can be detected by assessing intracellular events downstream of the target of the compound. In this way a single measurement is capable of reporting on a large number of upstream events.

FIG. 3. Cellular networks are controlled by molecules that undergo transitions. A molecule may have any number of states within a cell. A molecule starts its life cycle by being synthesized from its precursors or transported into the cell; then it undergoes a series of transitions such as those shown here.

FIG. 4 Examples of transitions which a molecule may undergo in a cell. A transition of a molecule is not associated with a specific subcellular compartment; instead, its compartment is determined by its interacting states (see FIG. 5). Drugs and toxicants affect transitions within those pathways that are either directly or indirectly affected by the drug or toxicant. Transitions of molecules are often measurable within cells.

FIG. 5. An illustration of the basic features of the ontology used herein. States and transitions (represented by circles and rectangles) can be used to assess pharmacological effects according to the present invention. Interactions (represented by lines) are also measurable transitions; alternatively, the complexes that result from biomolecular interactions represent new states that can be measured in cells.

FIG. 6. Ontology of a canonical pathway. In this diagram, one ‘module’ within a cellular network is shown, along with its interacting molecules and their associated states. Modules can range from individual molecules or genes, to a set of genes or proteins, or to functional subnetworks with definable cellular functions. Thus, a pathway may contain other pathways, and in turn may be a subset of another pathway. The context of this abstraction can therefore be extended to a complete network of all the interactions in a cell.

FIG. 7. A canonical pathway: EGF receptor signaling. Such pathway diagrams can be used in designing assay panels and in identifying potential molecular parameters suitable for pharmacological profiling. Examples of states and transitions, representing molecular parameters for which assays can be constructed in living cells, are provided.

FIG. 8. Steps in pharmacological profiling in cells. Key steps of the present invention are shown. The pharmacological profiles can be depicted in a variety of ways, for example, using a histogram; a matrix; a contour plot; or other suitable display method. In the matrix shown in the lower right, different individual drugs are on the x-axis and different assay/time/pretreatment conditions are on the y-axis. Green represents an increase in signal and red represents a decrease in signal in an assay. Such profiles are useful in comparisons, for example, in comparing a lead compound with a known drug or known toxicant or a compound previously shown to have adverse effects.

FIG. 9. Sample results for test compounds and reference compounds. Examples of pharmacological profiles for four different test compounds are shown, in addition to pharmacological profiles for ‘reference’ compounds: in this case, two known drugs and two known toxicants. Reference compounds can be selected whose biochemical, functional, cellular, physiological and/or clinical effects are well-characterized.

FIG. 10. Example of a high-throughput process for pharmacological profiling.

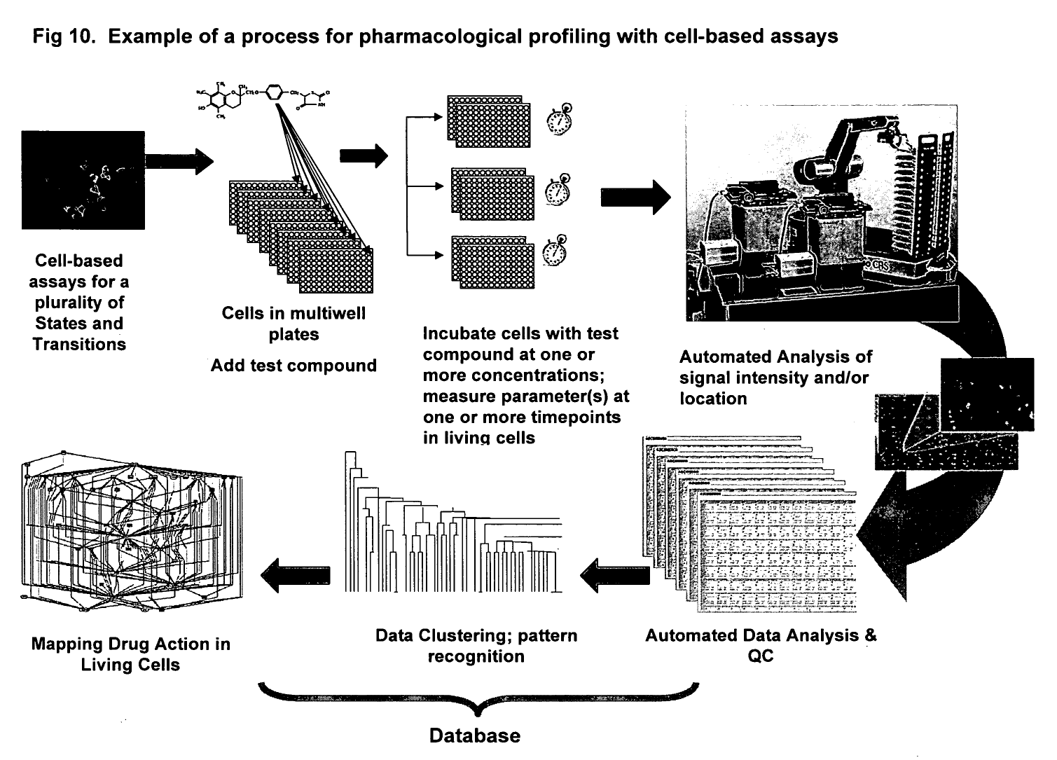

FIG. 11. Differential effects of EGF and drugs on signaling nodes in human cells. Representative photomicrographs show differential effects of EGF, EGF+PD98059, and EGF+SB203580 in human cells. Pathway activity of the test agents was assessed by measuring the subcellular compartmentation and quantity of phospho-ERK, phospho-Hsp27, and phospho-CREB. Values are presented as a ratio of total signal relative to the untreated control.

FIG. 12. Pharmacological profiles for EGF and two different kinase inhibitors based on their cellular activities. Histograms are shown representing quantitative results based on the images in FIG. 8. Unique profiles were generated for the different compounds reflecting their different mechanisms of action.

FIG. 13. Drug effects on network nodes in live cells. Representative fluorescence images are shown for 21 assays (from a total of 49 assays screened) in the presence of vehicle alone (left panels) and following treatment with the indicated drugs (right panel). Drugs and treatment times (in minutes) were as indicated in the right-hand image of each pair of images.

FIG. 14. Known drugs and toxicants have a range of stimulatory and inhibitory activities across network nodes in human cells. Two-dimensional matrix of drug effects on protein complex dynamics. Rows, 49 PCAs at individual time points; columns, 107 drugs at specified doses (ref table 2). Quantitative results are displayed clustered in both directions. For visualization purposes, only the 2 greatest levels of decrease or increase were represented as 2 shades of red or green, respectively. The brightest shade represents the most pronounced changes.

FIG. 15. Two-dimensional hierarchical clustering based on network activity. (A) Cluster dendrograms reveal drugs that have similar patterns of cellular activity. 107 commercially available drugs were clustered based upon their cellular activities in 49 distinct assays at multiple time points. Effects were measured as differences in fluorescence intensity within the cell or within defined cellular subregions according to the nature of the interaction. Functional drug classes are color coded as follows:

| | | | |
|---|---|---|---|
| cox-2 inhibitors | antipsychotics | statins | PDE-5 inhibitors |
| steroid receptor | proteasome | GPCR | Hsp90 inhibitors |
| related compounds | inhibitors | modulators | |
| PPAR-gamma | beta-adrenergic | HDAC | |
| agonists | receptor agonists | inhibitors | |

(B) Chemical structures of compounds illustrating examples of functional similarities among structurally similar and divergent drugs.

FIG. 16. Assay activity histograms highlighted drug similarities and differences. Each bar represents the activity of the drug on the indicated PCA at a single time point. Similar patterns of activity can be seen between Hsp90 inhibitors, 17-AAG (17-allylamino-17-demethoxy-geldanamycin) and geldanamycin, at 480 minutes.

FIG. 17. Off-pathway effects of β-adrenergic receptor agonists and the TNFα-family ligand TRAIL, at 480 minutes. Agonist-specific effects on the GPCR assays are highlighted with boxes.

FIG. 18. On-pathway verses off-pathway effects of the PPARγ ligands, GW1929, rosiglitazone, and pioglitazone, at 480 minutes; ligand-specific activity was detected on the RXRα:PPARγ assay, while only GW1929 had an effect on the Pin1:Jun assay.

FIG. 19. Assay activity histograms highlighted similarities and differences between the HMG-CoA reductase inhibitors. Histograms represent changes, relative to control, in the measured fluorescence signal. Each bar represents the activity of the indicated drug at a single time point. (A) Similar inhibitory effects of statins following isoproterenol stimulation of the βArr2:β2AR assay (as described in FIG. 1). The geranyl-geranyl transferase inhibitor, GGTI-2133, had no effect, while the farnesylation inhibitor, L-744,832, had similar activity to the statins. (B) Effects of statins on the Pin:Jun assay. Only cerivastatin and fluvastatin inhibited this network node.

FIG. 20. Results of 25-assay panel demonstrating hierarchical clustering of siRNAs based on their network activity in human cells. (A) Each column in the matrix corresponds to an individual siRNA pool, each row is a distinct assay representing a network node. Each data point within the matrix is color coded to illustrate relative differences within an assay. For each row, the dynamic range of the values (reported as log ratio of sample/control) is separated into 9 levels. An increase relative to the control value is displayed as green and a decrease is displayed as red. Each color is further divided into four levels: level 1 (>75%), level 2 (>=50% and <75%), level 3 (>=25% and <50%), level 4 (>0 and <25%). Level one is displayed as the brightest hue and level 2 as the darker. Levels 3 and 4 are shaded in black. (B) Selected clusters for siRNAs demonstrating expected ‘on-pathway’ effects are shown.

FIG. 21. Effects of 107 targeted siRNA pools on a single network node (Akt1:Hsp90-beta) in human cells. Inhibition of the PCA relative to the control is displayed to the left of the y-axis, while stimulation is displayed to the right. The siRNAs were grouped by common pathway or function and are color coded as follows (from bottom to top): ▪ PI3K/Akt, ▪ Hsp90 complex chaperones, U apoptotic regulators, ▪ NFkB pathway components, a nuclear hormone receptors and co-activators, a cell cycle and DNA damage response, ▪ Ras/MAPK signaling, ▪ RhoA family members and effectors, ▪ JNK/SAPK pathway, ▪ Wnt pathway, GPCR/G-proteins, ▪ PP2A phosphatase subunits, and ▪ PKA/PKC signaling. The 107 siRNAs represented in this figure are listed in order in Table S1 (B-E). Representative images of the effects of four siRNA SMART pools on the Akt1:Hsp90□ assay are shown. (B) control siRNA IX, (C) siCHEK2, (D) siHSPCB (Hsp90-beta) and (E) siAKT1.

FIG. 22. Effects of silencing Cdc37 on 25-assay panel. (A). Quantitation of the effect of siCDC37 on fluorescence intensity for each of 25 assays is represented as percent of control. Results for assays inhibited by ≧50% are depicted in magenta. (B-D). Representative images for the effects of siCdc37 on three assays relative to a control siRNA: (B) H-Ras:Raf, (C) Chk1:Cdc25C (+CPT), and (D) Akt1:p70S6K. (E) Representative images for the effects of the Cdc37 SMART pool components on the Akt1:p70S6K assay, where (C) represents treatment with the siRNA control, (P) shows the effect of the SMART pool, and (1-4) indicate the four component Cdc37 siRNAs.

FIG. 23. Illustration of the effect of H-Ras siRNA on cellular signaling nodes. The relationships of a subset of protein-protein interactions assayed in the siRNA screen are displayed in the context of known signaling pathways. Block arrows between proteins indicate the protein-protein interactions interrogated. Red arrows represent those PCAs whose fluorescence intensity was reduced by ≧50% by co-transfection with H-Ras siRNA. Representative images of specific PCAs inhibited by H-Ras siRNA (S) are shown relative to the corresponding siRNA control treatment (C). The Akt assays (Akt1:Hsp90-beta and Akt1:p70S6K) fell just below the cut-off, at 51% and 53% inhibition, respectively. Images from the Stat1:Stat1 assay, which was unaffected by H-Ras siRNA, are shown for comparison.

FIG. 24. Link between c-src and PPAR-gamma in human cells. (A) Co-transfection of c-src siRNA increases the PPAR-gamma:SRC-1 signal, both in the presence (right) and absence (left) of stimulation with 15 micromolar rosiglitazone for 90 minutes. (B and C) A chemical inhibitor of c-Src family kinases (PP2) produces an effect comparable to c-src siRNA. Representative images of drug effects are shown. (C) Data plotted for each drug treatment represent the mean (PPM) and standard error from 4 replicate wells in a minimum of two independent experiments. The effect of PP2 was highly significant (p<0.0001) relative to the DMSO control. (D) Quantitation of inhibition of each target mRNA (PPAR-gamma, EGFR, and c-Src) was performed with bDNA probes (GenoSpectra) designed for each target gene. Percent inhibition is normalized to the effects of the pooled negative control siRNAs. (E) Western blot of phosphorylation status of p44/42 MAPK/ERK in HEK293 cells stimulated with EGF (Lane 1) or rosiglitazone (Lanes 2-6), in combination with PP2, PP3, PD 153035 or PD 98059. (F) Hep3B cells were treated with PPAR-gamma agonists rosiglitazone, troglitazone and ciglitazone (50 micromolar each) for the indicated times. The phosphorylation status of p44/42 MAPK/ERK was compared to that of unstimulated (basal) or vehicle-treated (DMSO) cell extracts.

### DETAILED DESCRIPTION OF THE INVENTION

Principles of the Invention

Late-stage attrition of drug candidates costs the pharmaceutical industry billions of dollars, as evidenced by the withdrawals or recalls of many marketed drugs including Vioxx, Baycol, and Rezulin, due to adverse effects in man; and the failure of many otherwise-promising lead candidates due to toxicity in the clinical setting. An understanding of the full spectrum of biological activity of a new chemical entity would help to identify potentially adverse effects of drugs prior to clinical trials.

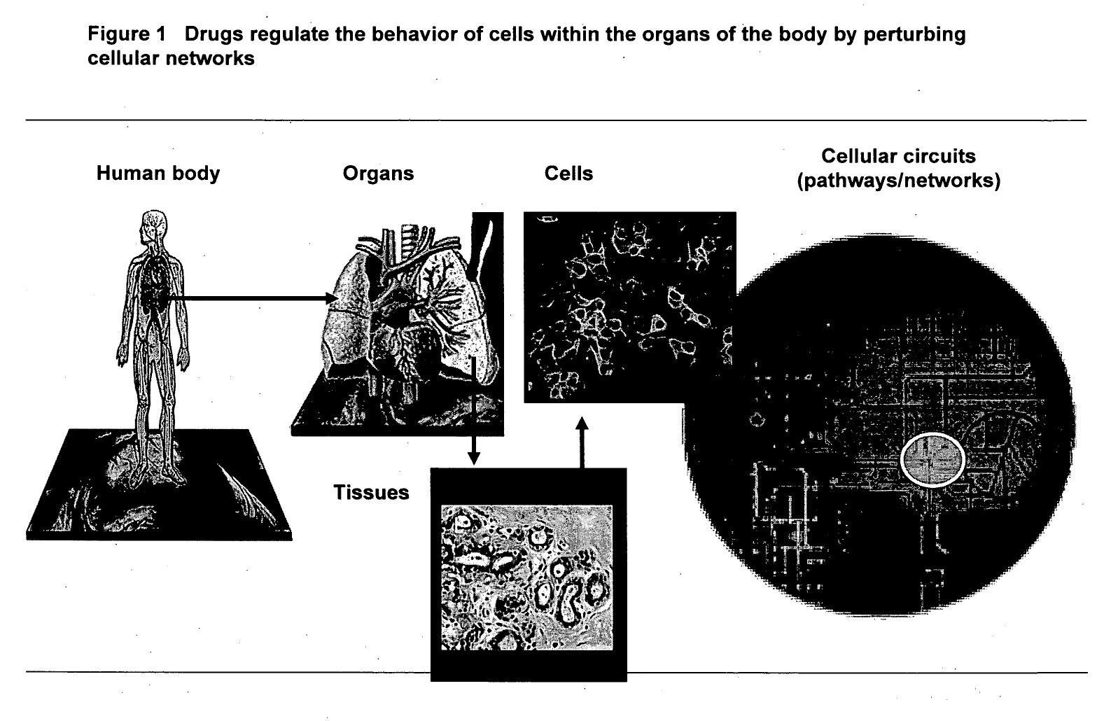

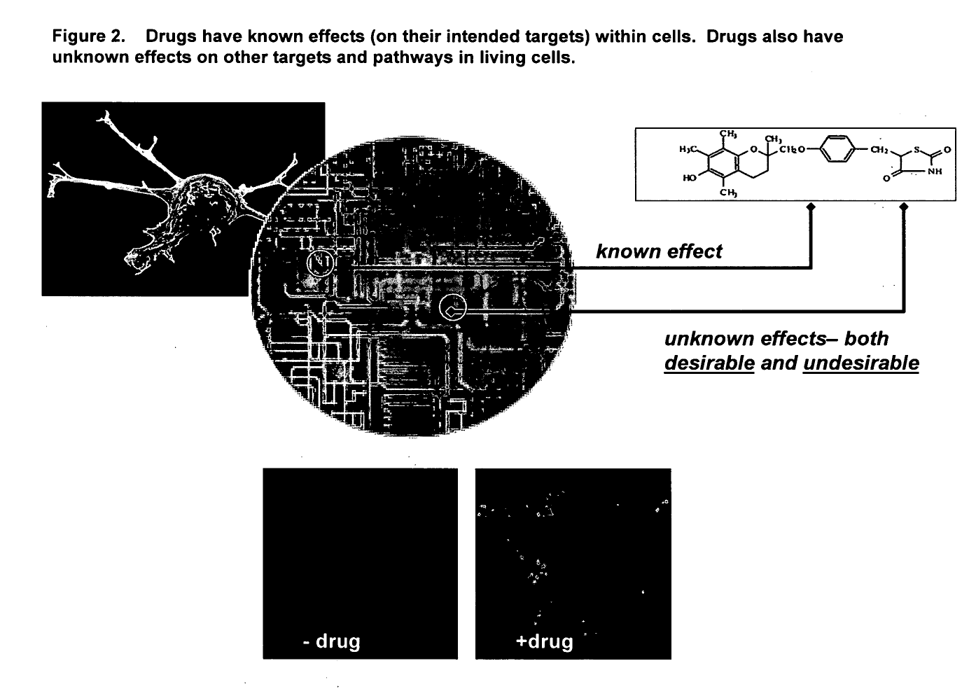

Drugs, when administered to patients, enter the circulation and—if they have adequate pharmacokinetic properties—reach various organs and tissues and cells of the body (FIG. 1). Ultimately, drugs act upon cells, which are the smallest living components of the human body. Regardless of whether a drug or drug candidate is an agonist, antagonist, inhibitor or activator of a target, drugs exert their actions by binding to a target protein and altering the function of that protein. For the purposes of this invention, we define ‘off pathway’ activity as any activity of a compound on a cellular target or pathway other than the intended target of the compound (see FIG. 2). These off-pathway effects can be desirable or undesirable. Desirable off-pathway effects are those that contribute to the efficacy of a drug or have positive secondary consequences; for example, strengthening bones while also lowering cholesterol; or, ameliorating chronic pain and also preventing cancer. Undesirable off-pathway effects are those that contribute to the toxicity or long-term adverse effects of a drug. In drug discovery it would be useful to be able to engineer-in desirable properties, and engineer-out any undesirable ones.

To address this problem on a large scale, we have developed a network biology-based platform for drug discovery and pharmacological profiling.

In general terms, a network is a system of components, or nodes, connected by physical or functional interactions (A. L. Barabasi and A. N. Oltvai, 2004, “Network Biology: Understanding the cell's functional organization”, in: Nature Reviews Genetics 5: 101-113). Networks of cells are controlled by physical interactions of various molecules that control specific aspects of cell behavior, metabolism and/or gene expression. Recent studies suggest that most networks within the cell approximate a ‘scale-free’ topology. Scale-free networks, which include the world-wide web as well as protein interaction networks, show remarkable robustness in that they remain functioning even if a large number of nodes have been inactivated. This is due to a considerable degree of redundancy within these networks which means that a given node can be reached or activated via more than one pathway. As a result, inactivation of an upstream node is likely to be compensated by the use of an alternative pathway, i.e. by the functional rewiring of the network. Importantly, cellular networks have a disproportionate number of highly connected nodes which means that a single node can, in principle, report out the activity of a plethora of upstream events.

We show that a single node can report out the activity of a drug on any number of upstream targets that are physically linked to the node. Moreover, we show that these events can be monitored in real time in intact cells. Therefore, by analyzing the effects of a test compound on diverse nodes representing a plurality of pathways across the cell, the entire spectrum of drug activity can be identified. The resulting profile or fingerprint of activity can be compared with that of well-characterized drugs and toxicants, enabling unintended, desirable or undesirable effects can be seen. Lead compounds can be prioritized, optimized (by chemical modification to remove undesired properties) or shelved (‘attrited’) based on their activity profiles. Through iterative cycles of lead synthesis and profiling, the invention can be used to optimize lead compounds by engineering-in desirable properties and engineering-out undesirable properties. Importantly, the process is sufficiently scalable to allow profiling of thousands of compounds across an entire cell.

Gene, Interaction, and Pathway Ontologies

The current state of knowledge on cellular regulatory pathways and larger networks is still fragmented, incomplete, and uncertain in many respects. Bioinformatics scientists are moving towards the direction of creating tools, languages and software for the integration of heterogeneous biological data and their analysis at the level of cellular systems and beyond. This direction requires establishing appropriate ‘ontologies’ to annotate the various parts and events occurring in the system. An ontology is a set of controlled and unambiguous vocabulary for describing objects and concepts. Pathway ontologies are useful in describing and applying the principles and methods of the present invention. These tools can be used to select pathways, nodes, and molecules for pharmacological profiling in cells. Many of these tools are known by those skilled in the art but will be described briefly here. It should be emphasized that the data underlying various databases have varied quality and accuracy; for complete information, and precise details on individual molecules and their functions, the biochemical literature should be consulted.

At the genome level, the Gene Ontology (GO) Consortium (www.geneontology.org) introduced a comprehensive ontology that is aimed to cover genes in all organisms. GO provides unique identifiers for each concept related to “molecular function”, “biological process” and “cellular component” searchable through the AmiGO tool (http://www.godatabase.org). Note that these three concepts (especially the concept of “biological process”) can be interpreted in terms of memberships of genes in cellular pathways; hence GO can be considered as part of a pathway ontology.

Among cellular pathways, metabolic pathways are generally more detailed and structured because of more advanced knowledge about metabolism in cells. In all of these databases, the proteins are classified according to the Enzyme Commission list of enzymes (EC numbers). These metabolic databases have strict ontologies which are focused on protein activities relevant to metabolic pathways.

There are a number of whole-cell modeling and simulation software environments (e.g. Virtual Cell, E-Cell and CellWare) with their own specific ontologies. These tools may be helpful in selecting pathway nodes and understanding pathway organization.

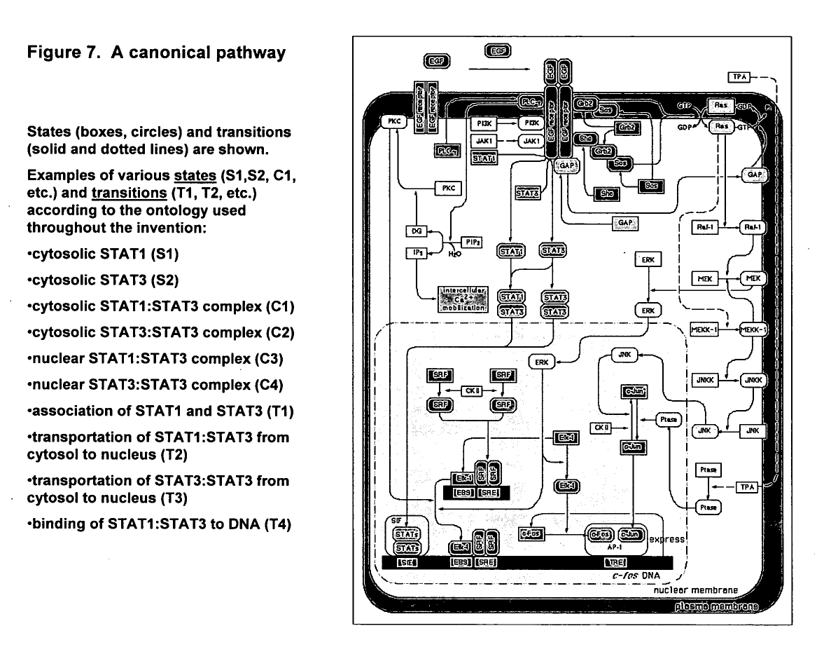

A conventional approach for representing cellular pathways is the use of diagrams or maps such as those found in the websites of ACSF, BioCarta and STKE (see Table 1). A map of a canonical pathway is also shown in FIG. 7. Pathway diagrams are not uniform and consistent among different websites; this is because the various representations carry implicit conventions rather than explicit rules as required by formal ontologies. Pathways databases can be classified into four groups as listed in Table 1. The first group of databases represents binary interaction databases that provide diverse amounts of data that can be used for selecting nodes to be tested within pathways. BIND, DIP, and MINT document experimentally determined protein-protein interactions from peer-reviewed literature or from other curated databases. BIND and MINT store experimental conditions used to observe the interaction, chemical action, kinetics and other information linked to the original research articles.

Databases of pathway diagrams provide a broad introductory view of cell regulatory pathways along with reviews and links. ACSF, STKE and Biocarta are comprehensive knowledge bases on signal transduction pathways and other regulatory networks. Metabolic signaling databases contain detailed information on metabolic pathways. These databases have well established data structures but have non-uniform ontologies. Enzyme catalyzed reactions, or the gene that encodes that enzyme or the structures of chemical compounds in pathways and reactions, can be displayed by BioCyc ontology based software for a given biochemical pathway. In addition BioCyc supports computational tools for simulation of metabolic pathways.

KEGG is a frequently-updated group of databases for the computerized knowledge representation of molecular interaction networks in metabolism, genetic information processing, environmental information processing, cellular processes and human diseases. The data objects in the KEGG databases are all represented as graphs and various computational methods for analyzing and manipulating these graphs are available.

The fourth category of the databases and software platforms listed in Table 1 is concerned with regulatory networks. GeneNet, aMAZE and PATIKA possess similar ontologies for representing and analyzing molecular interactions and cellular processes. PATIKA and GeneNet provide graphical user interfaces for illustrating signaling networks. The aMAZE tool called LightBench allows users to browse information stored in the database which covers chemical reactions, genes and enzymes involved in metabolic pathways, and transcriptional regulation. Another aMAZE tool called SigTrans is a database of models and information of signal transduction pathways. Cytoscape and PathwayAssist are similar software tools for automated analysis, integration and visualization of protein interaction maps. In these tools, automated methods for mining PubMed and other public literature databases are incorporated to facilitate the discovery of possible interactions or associations between genes or proteins. All of these resources may be useful in selecting pathways and nodes for pharmacological profiling according to our invention.

Application of Pathway Ontology to Pharmacological Profiling

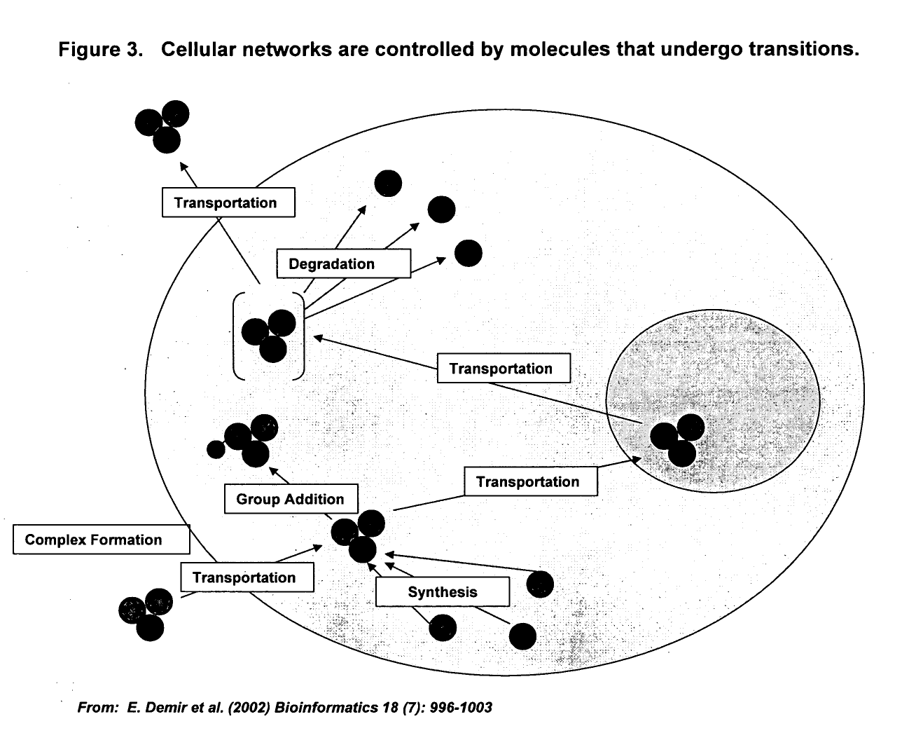

The use of a particular ontology is not intended to be limiting for the present invention, rather, it is intended as an aid in establishing a set of controlled and unambiguous vocabulary for describing concepts and methods of the invention. The ontology we have applied for this purpose has been recently implemented in a pathway database tool named PATIKA (Pathway Analysis Tool for Integration and Knowledge Acquisition) (www.patika.org). FIGS. 3-6 illustrate the basic features of the PATIKA ontology. Cellular networks are controlled by molecules, including macromolecules (e.g. DNAs, RNAs, proteins) and small molecules (e.g. ions, GTP, ATP), as well as physical events (e.g. heat, radiation, mechanical stress). The current invention is focused on molecular events; that is, changes that can be ascribed to a particular molecule or a set of molecules. Using the ontology described here, pathways are composed of components (states) and steps or processes (transitions). As shown in FIG. 3, a molecule starts its life cycle by being synthesized from its precursors or transported into the cell; then it undergoes a series of transitions. Each transition changes the information carried by the molecule, transforming it into a new state such as a phosphorylated state of a protein or a certain splice form of an RNA molecule. In this ontology, a state can be a macromolecule, a small molecule, or a physical complex. Moreover, a single molecule may have any number of states within a cell. For example, as depicted in FIG. 7, the well-characterized c-Jun protein exists in many different states including its native, phosphorylated, nuclear, Fos-associated, and DNA-bound forms. The transcription factors Stat1 and Stat3 also exist in various states, including their native states, cytosolic, nuclear, and complexed forms.

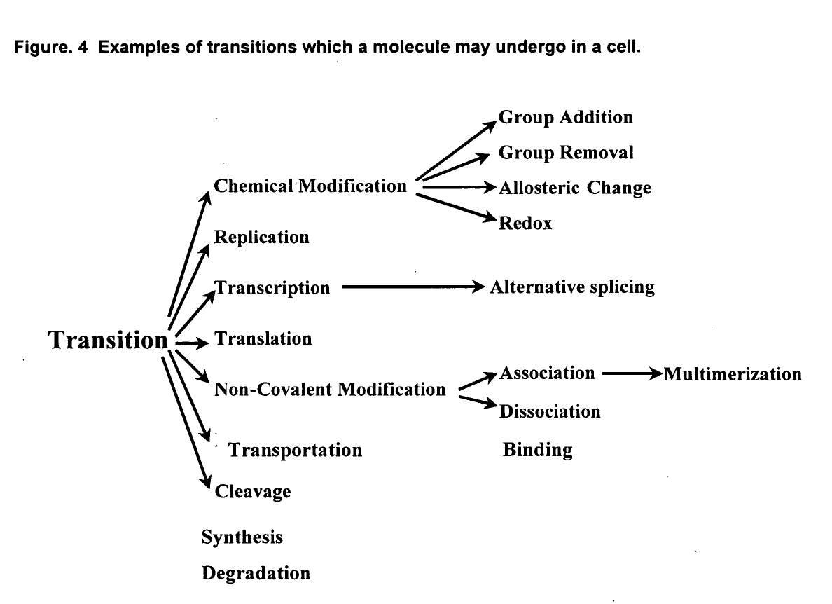

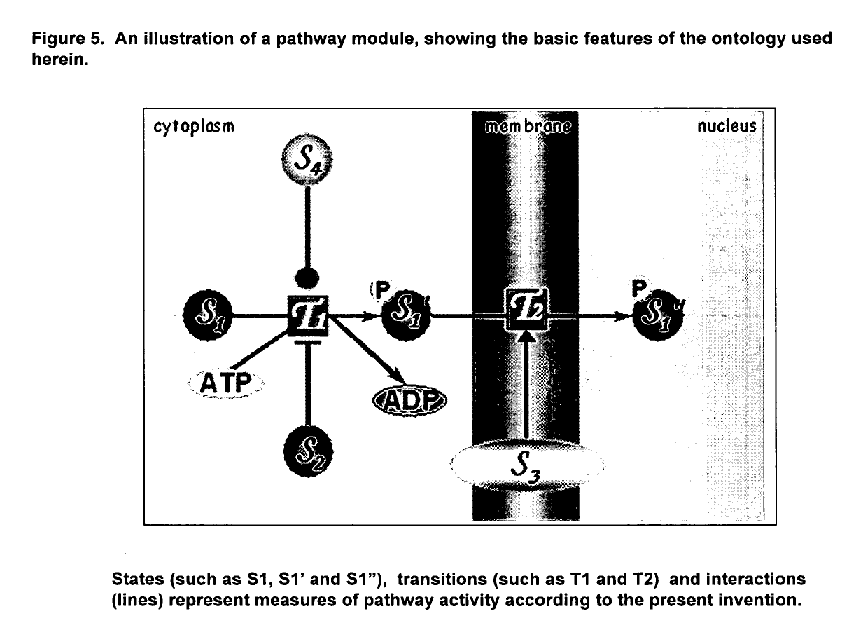

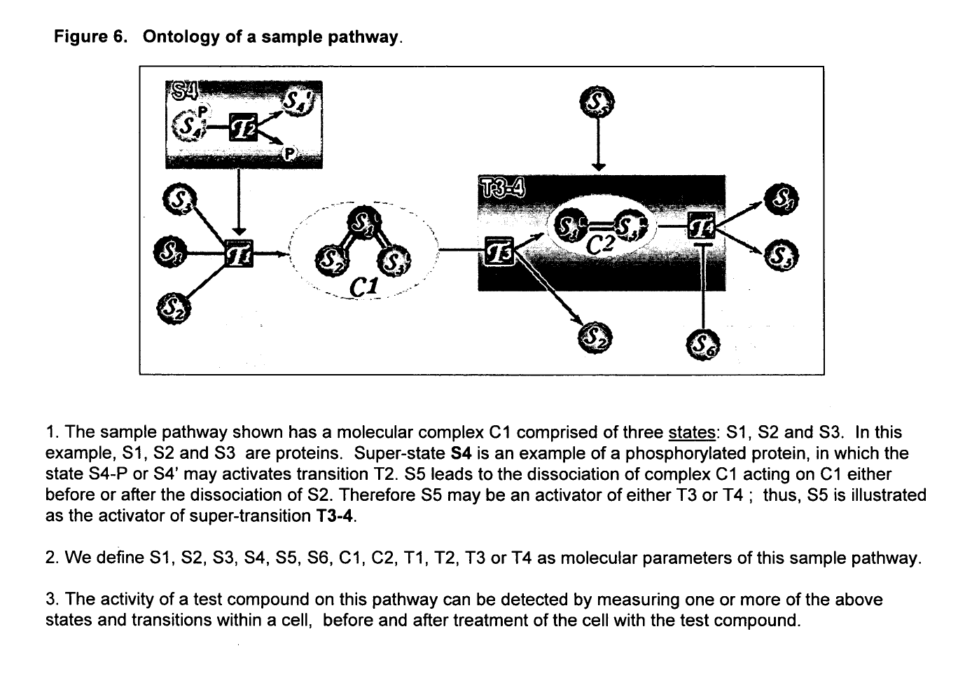

States are represented as nodes in a network (FIGS. 5-6) while maintaining their biological or chemical identities under a common molecule. A molecule may go through a certain set of possible transitions under a specified physiological condition—such as treatment of a cell with a drug or toxicant—and a totally different one under another condition, such as treatment of a cell with a different drug or toxicant. A state may go through a certain transition, may be affected by a transition, or may affect a transition as an activator or inhibitor. Thus, a transition has a number of associated states, which may be products, substrates or effectors of the transition. Transitions are depicted in the tree shown in FIG. 4. This model is very similar to the chemical equation: 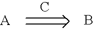
wherein A is a substrate, B is a product and C is an effector. This analogy is useful because most of the biological reactions of a cell are essentially chemical reactions. It is also an important concept because it is, in principle, possible to assess the occurrence or extent of the reaction by measuring some change in either substrate (A), product (B), or effector (C). Alternatively it may be possible to measure a change in the association or dissociation of an A-B-C complex. This flexibility in choice of measurement parameters is important because it provides flexibility in assay design to ensure optimal detection of the reaction.

This ontology can also be used to describe non-chemical phenomena such as transportation. In the example provided in FIG. 5, the molecule S1 (for example, a protein) has 3 states (namely, S₁, S₁′ and S₁″) located in two distinct subcellular compartments (cytoplasm and nucleus) which are separated by a third compartment, the nuclear membrane. S₁ and S₁′ are both in the cytoplasm. S₁ is phosphorylated through transition T₁ giving rise to a new state, the S₁′. S₁′ is translocated to the nucleus through transition T₂ and becomes S₁″. T₁ has two effector states, S₂ (inhibitor) and S₄ (unspecified effect). T₂ has an activator type of effector (S₃) representing, for example, the nuclear pore complex.

In biological systems, molecules often form complexes in order to perform certain tasks (FIG. 6). Each member of a molecular complex can be considered as a new state of its associated molecules. The intrinsic binding relationships affect the function of a molecular complex. Moreover, members of a molecular complex may independently participate in different transitions; thus one should be able to address each member individually. For example, one protein within a macromolecular complex may be post-translationally modified upon pathway activation; whereas another protein in the same complex may not be modified. The first protein then has unmodified and modified states when it is part of the complex. A molecular complex may also contain members from neighboring compartments (e.g. receptor-ligand complexes).

Transitions include transport of individual molecules as well as biomolecular complexes between cell compartments. The set of transitions that a state can be involved in is strictly related to its compartment; accordingly, the state's compartment is a part of the ontology. In order to reflect these changes a particular state is associated with exactly one compartment such as cell membrane, cytosol, nucleus or mitochondria. A transition is not associated with a specific subcellular compartment; instead, its compartment is determined by its interacting states as shown in FIG. 5. As the compartments are cell-type dependent, compartmental structure can be included in the ontology. For mammalian cells such well-known compartments include cytosol, nucleus, Golgi, lysosomes (endosomes) and mitochondria.

A set of transitions can be described as a single process (such as for the pathways listed in Table 2) and a set of related processes may be classified under one cellular mechanism (e.g. apoptosis). Some explicit examples of such ‘abstractions’ are shown in FIG. 6 where S₁, S₂ and S₃ are different proteins that undergo a transition (T1) to form a molecular complex C1. In this case the transition T1 is an interaction or association. State S4 is a phosphorylated protein, in which the State S₄-P or S₄′ may act as an activator of Transition T₂. S₅ leads to the dissociation of complex C1 acting on either before or after the dissociation of S₂. Therefore S₅ may be an activator of either T₃ or T₄ such that S₅ is illustrated as the activator of super-Transition T3-4. Such pathway modules can range from individual molecules or genes, to a set of genes or proteins, or to functional subnetworks with definable cellular functions. The context of these abstractions can therefore be extended to a complete network of all the interactions in a cell. A number of pathways that are well known to those skilled in the art are listed in Table 2. A pathway may contain other pathways, and in turn may be a subset of another pathway.

FIG. 7 is a diagram of the EGF receptor signaling pathway, which is a canonical pathway for which many of the participating proteins, their states, and their transitions, have been characterized. The diagram of depicts numerous states and transitions, where the participating states include macromolecules (proteins and DNA) and small molecules (guanine nucleotides, inositol trisphosphate, calcium, phosphate, EGF). Four cellular compartments are shown in the diagram: plasma membrane, cytosol, nuclear membrane, and nucleus. Different states are also shown for various components, for example, the different states of Stat1 include cytosolic Stat1; cytosolic Stat1:Stat3; nuclear Stat1:Stat3; and DNA-bound Stat1:Stat3. Jun and phospho-Jun are different states of the c-Jun protein. Transitions that are shown in FIG. 7 include binding (of EGF to its receptor); nucleotide hydrolysis (of GTP to GDP), second messenger release (of IP3, leading to calcium mobilization), associations (of Stat1 with Stat3, Stat 3 with Stat 3, JAK1 with EGF receptor, etc.), transportation (of ERK, Stat1:Stat3 and Stat3:Stat3 from cytosol to nucleus); phosphorylation and dephosphorylation (of c-Jun), protein:DNA binding (of Stats, Elk-1, and the AP-1 [Fos:Jun] complex), hydrolysis (of PIP2, by PLC-gamma) and many other examples of the transitions listed in FIG. 4. Many, if not all, of these events are dynamic which means the transitions occur in response to extrinsic signals such as treatment of the cell with EGF or with a drug or toxicant.

With this ontology in hand, the present invention provides for pharmacological profiling in living cells by measuring a plurality of states and transitions within a cell following treatment with the drug or other compound of interest. Collectively, we refer to states and transitions as ‘molecular parameters’ of pathways. If the molecular parameters represent dynamic network nodes they will report out the activity of numerous upstream events in the network. It should be emphasized that states and transitions of molecules that are useful in pharmacological profiling are those that respond dynamically to pathway modulators. So long as a molecular parameter is both dynamic (capable of being modulated by a cell treatment) and measurable (capable of being detected and quantified in an intact cell) then it may be used in pharmacological profiling according to this invention.

For a comprehensive picture of the potential activity of a compound, it would be ideal to be able to probe every pathway in a cell. The current state of our knowledge on cellular pathways is still fragmented, incomplete, and uncertain in many respects despite accumulating data. A general understanding of cellular pathways is taught to all biochemists, but that essential core knowledge can be supplemented by utilizing the vast biochemical literature to identify pathways that are considered to be distinct; constructing an assay(s) for that pathway based on a network node; identifying a molecular parameter in that pathway that is measurable using one or more of the methods provided herein; constructing a cell-based assay for that parameter; and then performing the assay in the absence and presence of a compound of interest to determine if the compound affects that parameter, which would indicate that the compound modulates the pathway. As a starting point in designing pathway-based panels for pharmacological profiling, there are a number of publicly available databases including those described in Tables 1, 3, 4, and 5 that can be used to aid in the selection of pathways and of potentially dynamic molecular parameters within the selected pathways.

Studies of pathways have traditionally focused either on the type of pathway (signal transduction pathways, biosynthetic pathways, processing pathways, etc.); the initiating stimulus (hormone, stress, growth factor, infectious agent, etc.); or on a series of molecules that are known to act in concert to transduce signals into the nucleus (JAK/STAT pathway, MAPK pathway, cAMP-dependent pathway, etc.) Some descriptions of mammalian cellular pathways are provided in Table 2. Since a compound of pharmacological interest may have an off-pathway effect on any of these pathways, assays for molecular parameters within these pathways may be constructed for pharmacological profiling according to this invention.

TABLE 1
Pathways databases and platforms

| | Database | Description | URL |
|---|---|---|---|
| Binary | BIND | Biomolecular interaction network database | http://www.bind.ca |
| interactions | BindingDB | Collection on experimental data on the | http://www.bindingdb.org |
| | | noncovalent association of molecules in solution | |
| | BRENDA | Enzyme Information System: sequence, | http://www.brenda.uni-koeln.de |
| | | structure, specificity, stability, reaction | |
| | | parameters, isolation data and molecular | |
| | | functions ontology | |
| | DIP | Database of interacting proteins | http://dip.doe-mbi.ucla.edu |
| | IntAct project | Public repository for annotated protein-protein | http://www.ebi.ac.uk/intact |
| | | interaction data | |
| | InterDom | Putative interacting protein domain database | http://interdom.lit.org.sg |
| | | derived from multiple sources | |
| | MINT | A molecular interaction database | http://mint.bio.uniroma2.it/mint/ |
| Static images | ACSF | Signaling resource for signal transduction | http://www.signaling-gateway.org/ |
| | | elements | |
| | BioCarta | Molecular relationship map pages from areas of | http://www.biocarta.com |
| | | active research | |
| | STKE | Signal transduction knowledge environment | http://stke.org/ |
| | HPRD | Human protein reference database | http://www.hprd.org/ |
| Metabolic | BRITE | Biomolecular relations in information | http://www.genome.ad.jp/brite |
| signaling | | transmission and expression | |
| | KEGG | Kyoto encyclopedia of genes and genomes: | http://www.genome.ad.jp/kegg |
| | | molecular interaction networks of metabolic and | |
| | | regulatory pathways | |
| | BioCyc | A collection of databases that describes the | http://biocyc.org/ |
| | | genome and metabolic pathways of a single | |
| | | organism | |
| | PathDB | A data repository and a system for building and | http://www.ncgr.org/pathdb |
| | | visualizing cellular networks | |
| Regulatory | aMAZE | A system for the representation, annotation, | http://www.amaze.ulb.ac.be/ |
| signaling | | management and analysis of biochemical and | |
| | | gene regulatory networks | |
| | Cytoscape | Software platform for visualizing molecular | http://www.cytoscape.org/ |
| | | interaction networks | |
| | GeneNet | Database on gene network components and a | http://wwwmgs.bionet.nsc.ru/mgs/gnw/genenet |
| | | program for the data visualization. | |
| | PATIKA | Software platform for pathway analysis tool for | http://www.patika.org/ |
| | | integration and knowledge acquisition | |
| | PathwayAssist | Tool for analysis, expansion and visualization of | http://www.ariadnegenomics.com/products/pathway.html |
| | | biological pathways, gene regulation networks | |
| | | and protein interaction maps | |
| | TRANSPATH | Gene regulatory network and microarray | http://www.biobase.de/pages/products/databases.html |
| | | analysis system. | |

TABLE 2
Examples of Cellular Processes and Associated Pathways

Acetylation and Deacetylation of RelA in The Nucleus
Actions of Nitric Oxide in the Heart
Activation of cAMP-dependent protein kinase, PKA
Activation of Csk by cAMP-dependent Protein Kinase Inhibits Signaling through the T Cell
Receptor
Activation of PKC through G protein coupled receptor
Activation of Src by Protein-tyrosine phosphatase alpha
Acute Myocardial Infarction
Adhesion and Diapedesis of Granulocytes
Adhesion and Diapedesis of Lymphocytes
Adhesion Molecules on Lymphocyte
ADP-Ribosylation Factor
Agrin in Postsynaptic Differentiation
Ahr Signal Transduction Pathway
AKAP95 role in mitosis and chromosome dynamics
AKT Signaling Pathway
ALK in cardiac myocytes
Alpha-synuclein and Parkin-mediated proteolysis in Parkinson's disease
Alternative Complement Pathway
Angiotensin II mediated activation of JNK Pathway via Pyk2 dependent signaling
Angiotensin-converting enzyme 2 regulates heart function
Anthrax Toxin Mechanism of Action
Antigen Dependent B Cell Activation
Antigen Processing and Presentation
Antisense Pathway
Apoptotic DNA fragmentation and tissue homeostasis
Apoptotic Signaling in Response to DNA Damage
Aspirin Blocks Signaling Pathway Involved in Platelet Activation
ATM Signaling Pathway
Attenuation of GPCR Signaling
B Cell Receptor Complex
B Cell Survival Pathway
B Lymphocyte Cell Surface Molecules
Basic mechanism of action of PPARa, PPARb(d) and PPARg and effects on gene expression
Basic Mechanisms of SUMOylation
BCR Signaling Pathway
Beta-Oxidation of Fatty Acids
Bioactive Peptide Induced Signaling Pathway
Biosynthesis of Arginine in Bacteria
Biosynthesis of Chorismate in Bacteria and Plants
Biosynthesis of Cysteine from serine in bacteria and plants
Biosynthesis of Cysteine in mammals
Biosynthesis of Glycine and Serine
Biosynthesis of isoleucine
Biosynthesis of leucine
Biosynthesis of Lysine
Biosynthesis of neurotransmitters
Biosynthesis of phenylalanine and tyrosine in bacteria and plants
Biosynthesis of Proline in Bacteria
Biosynthesis of spermidine and spermine
Biosynthesis of sphingolipids
Biosynthesis of threonine and methionine
Biosynthesis of Tryptophan in Bacteria and Plants
Biosynthesis of valine
Blockade of Neurotransmitter Relase by Botulinum Toxin
Bone Remodelling
BRCA1-dependent Ub-ligase activity
BTG family proteins and cell cycle regulation
Bystander B Cell Activation
Ca++/Calmodulin-dependent Protein Kinase Activation
Cadmium induces DNA synthesis and proliferation in macrophages
Calcium Signaling by HBx of Hepatitis B virus
Cardiac Protection Against ROS
CARM1 and Regulation of the Estrogen Receptor
Caspase Cascade in Apoptosis
Catabolic pathway for asparagine and asparate
Catabolic pathways for alanine, glycine, serine, cysteine, tryptophan, and threonine
Catabolic Pathways for Arginine, Histidine, Glutamate, Glutamine, and Proline
Catabolic Pathways for Methionine, Isoleucine, Threonine and Valine
CBL mediated ligand-induced downregulation of EGF receptors
CCR3 signaling in Eosinophils
CD40L Signaling Pathway
cdc25 and chk1 Regulatory Pathway in response to DNA damage
CDK Regulation of DNA Replication
Cell Cycle: G1/S Check Point
Cell Cycle: G2/M Checkpoint
Cell to Cell Adhesion Signaling
Cells and Molecules involved in local acute inflammatory response
Ceramide Signaling Pathway
Chaperones modulate interferon Signaling Pathway
ChREBP regulation by carbohydrates and cAMP
Chromatin Remodeling by hSWI/SNF ATP-dependent Complexes
Circadian Rhythms
Classical Complement Pathway
Comparison of Beta oxidation in mitochondria and peroxisomes and glyoxysomes
Complement Pathway
Control of Gene Expression by Vitamin D Receptor
Control of skeletal myogenesis by HDAC & calcium/calmodulin-dependent kinase (CaMK)
Corticosteroids and cardioprotection
CTCF: First Multivalent Nuclear Factor
CTL mediated immune response against target cells
CXCR4 Signaling Pathway
Cyclin E Destruction Pathway
Cycling of Ran in nucleocytoplasmic transport
Cyclins and Cell Cycle Regulation
Cystic fibrosis transmembrane conductance regulator and beta 2 adrenergic receptor pathway
Cytokine Network
Cytokines and Inflammatory Response
D4-GDI Signaling Pathway
Degradation of the RAR and RXR by the proteasome
Dendritic cells in regulating TH1 and TH2 Development
Deregulation of CDK5 in Alzheimers Disease
Dicer Pathway
Double Stranded RNA Induced Gene Expression
Downregulated of MTA-3 in ER-negative Breast Tumors
E2F1 Destruction Pathway
Effects of calcineurin in Keratinocyte Differentiation
EGF Signaling Pathway
Eicosanoid Metabolism
Electron Transport Reaction in Mitochondria
Endocytotic role of NDK, Phosphins and Dynamin
Eph Kinases and ephrins support platelet aggregation
EPO Signaling Pathway
ER-associated degradation (ERAD) Pathway
Erk and PI-3 Kinase Are Necessary for Collagen Binding in Corneal Epithelia
Erk1/Erk2 Mapk Signaling pathway
Erythrocyte Differentiation Pathway
Erythropoietin mediated neuroprotection through NF-kB
Estrogen-responsive protein Efp controls cell cycle and breast tumors growth
Eukaryotic protein translation
Extrinsic Prothrombin Activation Pathway
FAS signaling pathway (CD95)
Fc Epsilon Receptor I Signaling in Mast Cells
Feeder Pathways for Glycolysis
Fibrinolysis Pathway
fMLP induced chemokine gene expression in HMC-1 cells
Formation of Ketone Bodies from acetyl-CoA
FOSB gene expression and drug abuse
Free Radical Induced Apoptosis
Function of SLRP in Bone: An Integrated View
FXR and LXR Regulation of Cholesterol Metabolism
Gamma-aminobutyric Acid Receptor Life Cycle
GATA3 participate in activating the Th2 cytokine genes expression
Generation of amyloid b-peptide by PS1
Ghrelin: Regulation of Food Intake and Energy Homeostasis
Glycolysis Pathway
G-Protein Signaling Through Tubby Proteins
Granzyme A mediated Apoptosis Pathway
Growth Hormone Signaling Pathway
g-Secretase mediated ErbB4 Signaling Pathway
Hemoglobin's Chaperone
HIV Induced T Cell Apoptosis
HIV-1 defeats host-mediated resistance by CEM15
HIV-I Nef: negative effector of Fas and TNF
Hop Pathway in Cardiac Development
How does salmonella hijack a cell
How Progesterone Initiates the Oocyte Maturation
Human Cytomegalovirus and Map Kinase Pathways
Hypoxia and p53 in the Cardiovascular system
Hypoxia-Inducible Factor in the Cardiovascular System
IFN alpha signaling pathway
IFN gamma signaling pathway
IGF-1 Signaling Pathway
IL 17 Signaling Pathway
IL 18 Signaling Pathway
IL 2 signaling pathway
IL 3 signaling pathway
IL 4 signaling pathway
IL 5 Signaling Pathway
IL 6 signaling pathway
IL-10 Anti-inflammatory Signaling Pathway
IL12 and Stat4 Dependent Signaling Pathway in Th1 Development
IL-2 Receptor Beta Chain in T cell Activation
IL22 Soluble Receptor Signaling Pathway
IL-7 Signal Transduction
Inactivation of Gsk3 by AKT causes accumulation of b-catenin in Alveolar Macrophages
Induction of apoptosis through DR3 and DR4/5 Death Receptors
Influence of Ras and Rho proteins on G1 to S Transition
Inhibition of Cellular Proliferation by Gleevec
Inhibition of Huntington's disease neurodegeneration by histone deacetylase inhibitors
Inhibition of Matrix Metalloproteinases
Insulin Signaling Pathway
Integrin Signaling Pathway
Internal Ribosome entry pathway
Intrinsic Prothrombin Activation Pathway
Ion Channel and Phorbal Esters Signaling Pathway
Ion Channels and Their Functional Role in Vascular Endothelium
Keratinocyte Differentiation
Lactose Synthesis
Lck and Fyn tyrosine kinases in initiation of TCR Activation
Lectin Induced Complement Pathway
Leloir pathway of galactose metabolism
Links between Pyk2 and Map Kinases
Lissencephaly gene (LIS1) in neuronal migration and development
Low-density lipoprotein (LDL) pathway during atherogenesis
Malate-aspartate shuttle
Map Kinase Inactivation of SMRT Corepressor
MAPKinase Signaling Pathway
mCalpain and friends in Cell motility
Mechanism of Acetaminophen Activity and Toxicity
Mechanism of Gene Regulation by Peroxisome Proliferators via PPARa(alpha)
Mechanism of Protein Import into the Nucleus
Mechanisms of transcriptionalrepression by DNA methylation
Melanocyte Development and Pigmentation Pathway
Metabolism of Anandamide, an Endogenous Cannabinoid
METS affect on Macrophage Differentiation
Mitochondrial Carnitine Palmitoyltransferase (CPT) System
Monocyte and its Surface Molecules
Msp/Ron Receptor Signaling Pathway
mTOR Signaling Pathway
Multi-Drug Resistance Factors
Multiple antiapoptotic pathways from IGF-1R signaling lead to BAD phosphorylation
Multi-step Regulation of Transcription by Pitx2
Nerve growth factor pathway (NGF)
Neuropeptides VIP and PACAP inhibit the apoptosis of activated T cells
Neuroregulin receptor degredation protein-1 Controls ErbB3 receptor recycling
Neutrophil and Its Surface Molecules
NFAT and Hypertrophy of the heart (Transcription in the broken heart)
NFkB activation by Nontypeable Hemophilus influenzae
NF-kB Signaling Pathway
Nitric Oxide Signaling Pathway
Nitrogen-depedent regulation of Rtg1 and Rtg3 in TOR pathway
NO2-dependent IL 12 Pathway in NK cells
Nuclear receptors coordinate the activities of chromatin remodeling complexes and coactivators
Nuclear Receptors in Lipid Metabolism and Toxicity
Omega Oxidation
Opposing roles of AIF in Apoptosis and Cell Survival
Overview of telomerase protein component gene hTert Transcriptional Regulation
Overview of telomerase RNA component gene hTerc Transcriptional Regulation
OX40 Signaling Pathway
Oxidation of odd-numbered chain fatty acid, from Propionyl-CoA to Succinyl-CoA
Oxidation of Polyunsaturated Fatty Acid
Oxidative reactions of the pentose phosphate pathway
Oxidative Stress Induced Gene Expression Via Nrf2
p38 MAPK Signaling Pathway
p53 Signaling Pathway
PDGF Signaling Pathway
Pelp1 Modulation of Estrogen Receptor Activity
Pertussis toxin-insensitive CCR5 Signaling in Macrophage
Phosphatidylcholine Biosynthesis Pathway
Phosphoinositides and their downstream targets.
Phospholipase C d1 in phospholipid associated cell signaling
Phospholipase C Signaling Pathway
Phospholipase C-epsilon pathway
Phospholipid Biosynthesis in E. Coli Pathway
Phospholipids as signalling intermediaries
Phosphorylation of MEK1 by cdk5/p35 down regulates the MAP kinase pathway
PKC-catalyzed phosphorylation of inhibitory phosphoprotein of myosin phosphatase
Platelet Amyloid Precursor Protein Pathway
Polyadenylation of mRNA
Presenilin action in Notch and Wnt signaling
Prion Pathway
Proepithelin Conversion to Epithelin and Wound Repair Control
Proteasome Complex
Protein Kinase A at the Centrosome
Proteolysis and Signaling Pathway of Notch
PTEN dependent cell cycle arrest and apoptosis
Rab GTPases Mark Targets In The Endocytotic Machinery
Rac 1 cell motility signaling pathway
Ras Signaling Pathway
Ras-Independent pathway in NK cell-mediated cytotoxicity
RB Tumor Suppressor/Checkpoint Signaling in response to DNA damage
Reelin Signaling Pathway
Regulation of BAD phosphorylation
Regulation of cell cycle progression by Plk3
Regulation of ck1/cdk5 by type 1 glutamate receptors
Regulation of eIF2
Regulation of eIF4e and p70 S6 Kinase
Regulation of hematopoiesis by cytokines
Regulation of MAP Kinase Pathways Through Dual Specificity Phosphatases
Regulation of p27 Phosphorylation during Cell Cycle Progression
Regulation of PGC-1a
Regulation of Spermatogenesis by CREM
Regulation of Splicing through Sam68
Regulation of transcriptional activity by PML
Regulators of Bone Mineralization
Repression of Pain Sensation by the Transcriptional Regulator DREAM
Reversal of Insulin Resistance by Leptin
Rgt1 in Yeast Glucose Induction Pathway
Rho cell motility signaling pathway
Rho-Selective Guanine Exchange Factor AKAP13 Mediates Stress Fiber Formation
RNA polymerase III transcription
Role of BRCA1, BRCA2 and ATR in Cancer Susceptibility
Role of EGF Receptor Transactivation by GPCRs in Cardiac Hypertrophy
Role of ERBB2 in Signal Transduction and Oncology
Role of Erk5 in Neuronal Survival
Role of MAL in Rho-Mediated Activation of SRF
Role of MEF2D in T-cell Apoptosis
Role of Mitochondria in Apoptotic Signaling
Role of nicotinic acetylcholine receptors in the regulation of apoptosis
Role of Parkin in the Ubiquitin-Proteasomal Pathway
Role of PI3K subunit p85 in regulation of Actin Organization and Cell Migration
Role of PPAR-gamma Coactivators in Obesity and Thermogenesis
Role of Ran in mitotic spindle regulation
Role of β-arrestins in the activation and targeting of MAP kinases
Role of Tob in T-cell activation
Roles of β-arrestin-dependent Recruitment of Src Kinases in GPCR Signaling
SARS Coronavirus Protease
Segmentation Clock
Selective expression of chemokine receptors during T-cell polarization
Shuttle for transfer of acetyl groups from mitochondria to the cytosol
Signal Dependent Regulation of Myogenesis by Corepressor MITR
Signal transduction through IL1R
Signaling of Hepatocyte Growth Factor Receptor
Signaling Pathway from G-Protein Families
Skeletal muscle hypertrophy is regulated via AKT/mTOR pathway
Small Leucine-rich Proteoglycan (SLRP) molecules
Snf1 in Yeast Glucose Repression/Derepression
SODD/TNFR1 Signaling Pathway
Sonic Hedgehog (Shh) Pathway
Sonic Hedgehog (SHH) Receptor Ptc1 Regulates cell cycle
Spliceosomal Assembly
Sprouty regulation of tyrosine kinase signals
SREBP control of lipid synthesis
Beta-arrestins in GPCR Desensitization
Starch Synthesis
Stat3 Signaling Pathway
Stathmin and breast cancer resistance to antimicrotubule agents
Steps in the Glycosylation of Mammalian N-linked Oligosaccarides
Stress Induction of HSP Regulation
Sucrose Synthesis
SUMOylation as a mechanism to modulate CtBP-dependent gene responses
Sumoylation by RanBP2 Regulates Transcriptional Repression
Synaptic Proteins at the Synaptic Junction
Synthesis of Cardiolipin & phosphatidylinositol
Synthesis of plasmalogens
T Cell Receptor and CD3 Complex
T Cell Receptor Signaling Pathway
T Cytotoxic Cell Surface Molecules
T Helper Cell Surface Molecules
TACI and BCMA stimulation of B cell immune responses.
Telomeres, Telomerase, Cellular Aging, and Immortality
TGF beta signaling pathway
Th1/Th2 Differentiation
The 4-1BB-dependent immune response
The Citric Acid Cycle
The Co-Stimulatory Signal During T-cell Activation
The IGF-1 Receptor and Longevity
The information-processing pathway at the IFN-beta enhancer
The PRC2 Complex Sets Long-term Gene Silencing Through Modification of Histone Tails
The reactions that feed amino groups into the urea cycle
The Role of Eosinophils in the Chemokine Network of Allergy
The role of FYVE-finger proteins in vesicle transport
The salvage pathway from serine to phosphatidylcholine
Thrombin signaling and protease-activated receptors
TNF/Stress Related Signaling
TNFR1 Signaling Pathway
TNFR2 Signaling Pathway
Toll-Like Receptor Pathway
TPO Signaling Pathway
Transcription factor CREB and its extracellular signals
Transcription Regulation by Methyltransferase of CARM1
Transcriptional activation of dbpb from mRNA
Trefoil Factors Initiate Mucosal Healing
Trka Receptor Signaling Pathway
TSP-1 Induced Apoptosis in Microvascular Endothelial Cell
Tumor Suppressor Arf Inhibits Ribosomal Biogenesis
uCalpain and friends in Cell spread
VEGF, Hypoxia, and Angiogenesis
Visceral Fat Deposits and the Metabolic Syndrome
Visual Signal Transduction
Vitamin C in the Brain
West Nile Virus
WNT Signaling Pathway
Wnt/LRP6 Signalling
Y branching of actin filaments

Process of Pharmacological Profiling

Our invention teaches that, because of the physical connections of macromolecules within cellular networks, drug effects on a particular target will radiate to network nodes ‘downstream’ of the effect of the drug. These effects of drugs on pathways can be ‘captured’ at any specific point in time by measuring dynamic states and/or transitions within treated cells. If a panel is constructed comprising assays for a plurality of molecular parameters in whole cells, each of which faithfully reports out the activity of one or more signaling pathways at a particular point in time, then a profile of drug action in the cell can be assessed. We have found that these effects can indeed be readily and reliably captured in real time using modern instrumentation for cellular analysis. Virtually any state or transition will be suitable for application with this invention if it meets the following criteria: (a) a robust and quantitative assay can be constructed with an intact cell, at the molecular level, for that particular state or transition; and (b) there is a change in the state or transition in response to a compound of interest.

A very comprehensive panel of assays for pharmacological profiling may be constructed by using an assay for at least one parameter of each of the pathways listed in Table 2. However, this is neither practical nor necessary. Because pathways are often interconnected—exhibiting ‘cross-talk’—or sharing common signaling entities—it is not necessary to construct an assay for every parameter that may be directly or indirectly affected by a drug of interest. In one example provided herein we were able to distinguish between compounds acting on different pathways by using a panel of only three assays and measuring only three different states (phosphoproteins). In another example provided herein we distinguished and successfully grouped 98 different known drugs based on their activities in 49 assays. In the latter case the informativeness of the method was increased by performing the assays at different points in time following drug treatment such that drugs with short-term effects could be seen and could be distinguished from drugs with longer-term effects. In the latter study we also probed some network nodes under different pretreatment conditions, such as in the absence and presence of a known agonist of a particular pathway, such that either inhibition or activation of the node by a drug could be seen. Thus the use of time, dose, and pretreatment condition as variables can help to expand the informativeness of the results of the panels.

A panel can be comprised of as few as two distinct assays or as many as hundreds or thousands of distinct assays. The number of assays can be chosen by the investigator or determined empirically, and will depend on a number of factors, including the performance of any individual assay, the desired scope of the profile, and the identity of the compound of interest. The utility of the approach is based not on the number of parameters that are assayed but on the breadth of pathways covered. Adding more sentinels into the same pathways will help in defining novel mechanisms of action and in identifying potential new drug targets; but will not necessarily provide additional predictive power. Ultimately, a single informative sentinel for each cellular pathway is needed. It is possible that a completely predictive platform could be achieved with a panel of 200-500 assays. The biochemical literature, and our own experience, suggests that biochemical networks are highly ramified. For example, in 2003 we mapped all the interactions among over 160 cancer-related proteins, running approximately 56,000 interaction assays in the process. Our results showed an average hit rate of 5 interactions per protein; a number that is consistent with protein interaction maps of model organisms such as yeast. If one assumes 30,000 proteins in the human proteome (excluding splice variants, that is) then there may be around 6000 human protein-protein interactions that are physically separated by one or more degrees of separation (30,000/5). Finally, if we assume that each of 6000 non-redundant sentinels serves to report on the activity of 15 upstream events, then a collection of 400 sentinels would report out the activity of every pathway in the cell.

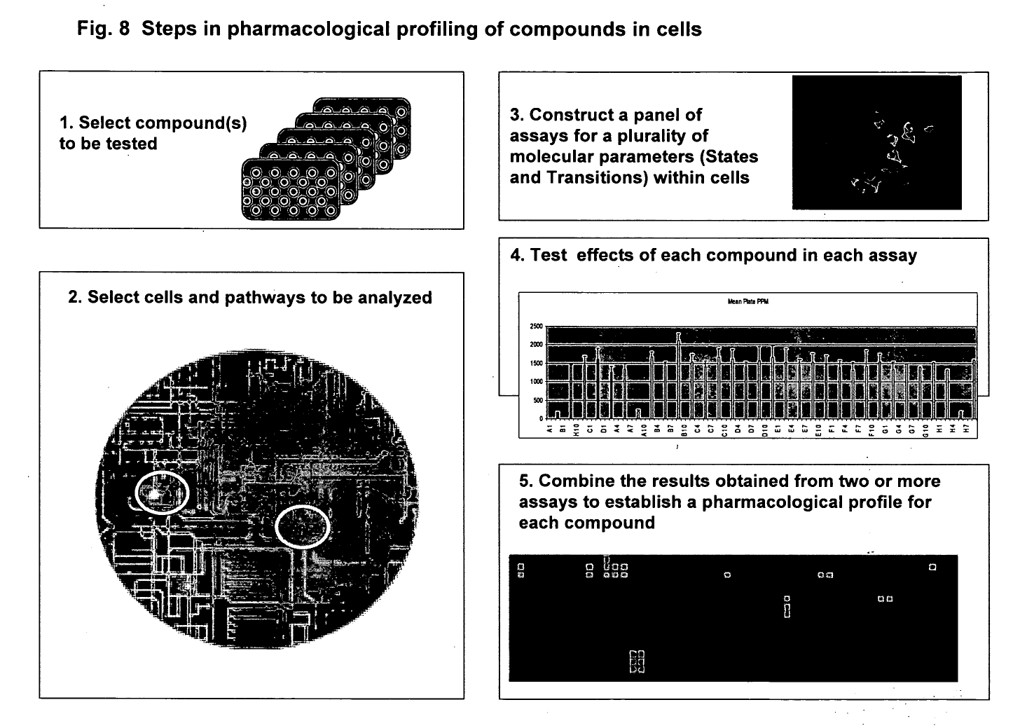

The steps in pharmacological profiling are shown in FIG. 8. First, a panel of cell-based assays for a plurality of states and transitions is constructed, wherein each assay is designed to measure a state or transition within a pathway of interest. The parameters for the assays in the panel can either be selected rationally—for example, by prior knowledge of a pathway or a protein—or empirically, through trial and error. Moreover, an unlimited number of assays can simply be constructed at random and tested empirically for their responsiveness to any number of drugs or chemical compounds and the results combined into a pharmacological profile. Second, for each assay the cells are contacted with a chemical compound of interest in a suitable vehicle, at a particular concentration, and for a pre-selected period of time. Preferably, positive and negative controls are run for each assay, at each time point and stimulus condition. Third, a molecular parameter (as described above and in the detailed description of the invention) is measured in the intact (live or fixed) cells. For each assay, the result for the test compound is compared to the result for untreated cells (vehicle alone) to establish the effect of the test compound on the test parameter. Finally, the results of a plurality of assays are combined to establish a pharmacological profile for the test compound. The resulting profiles may be displayed in a variety of ways. A simple histogram can be used to depict a pharmacological profile. In a preferred embodiment, the results of each screen are depicted in a color-coded matrix in which red denotes a decrease in signal intensity or location whereas green denotes an increase as shown here. Different shades of red and green can be used to depict the intensity of the change. A variety of visualization tools and third-party software can be used to display and analyze the profiles. The profiles, which serve as test compound ‘fingerprints’, can then be compared with the profiles established for reference compounds under identical conditions. These profiles can be used to identify compounds with desired functional profiles and to eliminate compounds with undesired profiles.

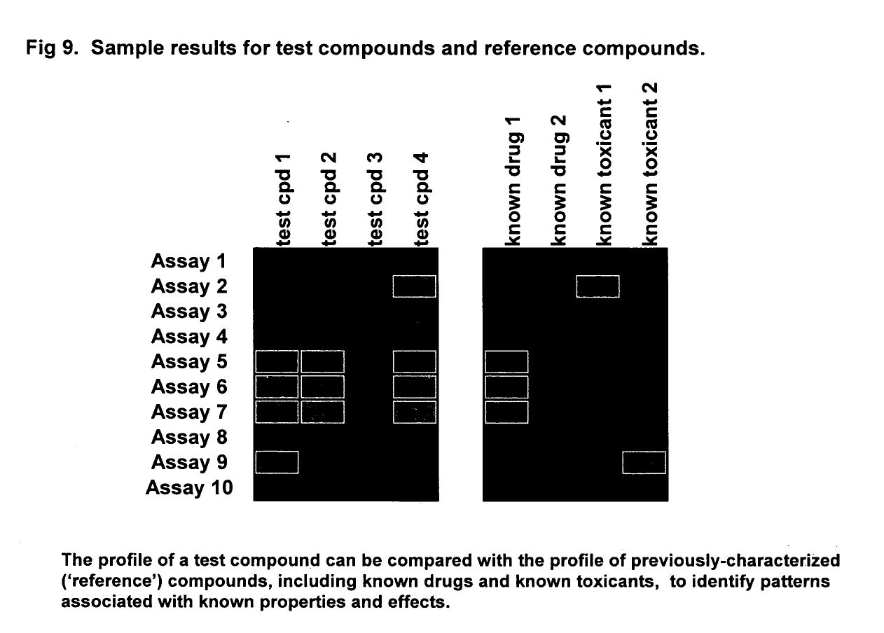

In the example shown in FIG. 9, each compound was assayed against a panel comprised of 10 different assays. Assay 1 represents a dynamic, measurable parameter of Pathway 1; Assay 2 represents a dynamic, measurable parameter of Pathway 2; etc. Black indicates no effect; green indicates a positive effect (increase or activation); and red indicates a negative effect (decrease or inhibition) of the measured parameter. Test compounds 1 and 2 are analogues, having the same core chemical structures. Test compound 1 has a positive effect on pathways 5, 6, and 7 as indicated by the green boxes in the matrix. Its profile is therefore similar to that of known drug 1, suggesting that—given satisfactory pharmacokinetic and pharmacodynamic properties—it may have desired properties similar to that of known drug 1. However, test compound 1 also has a negative effect on pathway 4 and a positive effect on pathway 9, a pattern similar to that of known toxicant 2, suggesting that test compound 1 may have similar toxic effects at the concentration tested. We sought to improve the properties of test compound 1 by synthesis of an analogue that has the desired properties of test compound 1 but not the undesired properties of known toxicant 2. Synthesis of an analogue of test compound 1 resulted in test compound 2 which has a profile of activity similar to known drug 1, but does not have the activity of known toxicant 2. Therefore, we are taking test compound 2 forward into drug development and shelving test compound 1. Test compounds 3 and 4 are from a different lead series and share a core chemical structure. Test compound 3 has the desired properties of known drug 2. Test compound 4 also shares those properties but is less specific, having activity on pathways that are targeted by known drug 1 and having the undesired activity of known toxicant 1. Consequently, we are taking test compound 3 forward into development and shelving test compound 4. The examples illustrate how pharmacological profiling can be used to guide drug discovery, in particular for lead optimization and attrition.

Any type of chemical compound, drug lead, known drug or toxicant of interest, target class or mechanism can be evaluated with the methods provided herein. In using the general term ‘compound’ or ‘test compound’ we include synthetic molecules, natural products, combinatorial libraries, known or putative drugs, ligands, antibodies, peptides, recombinant proteins, small interfering RNAs (siRNAs), toxicants, or any other chemical or biological agent whose activity is desired to be tested. Screening hits from combinatorial library screening or other high-throughput screening campaigns can be profiled in conjunction with the present invention.

Reference compounds may be chosen from a group of compounds that have established properties, such as well-characterized drugs, competitor compounds, known toxicants, or lead compounds from the same lead series as the test compound. In the example provided herein we used 98 different known drugs and toxicants in a reference set. In a preferred embodiment, reference compounds include known drugs and known toxicants. Known drugs can be obtained from commercial sources including (MicroSource) etc. Known toxicants can be obtained from chemical suppliers including Sigma Chemical Co. (St. Louis). These can be utilized as references at one or more concentrations in the assay panel; alternatively, a dose-response range can be constructed. The concentrations of known drugs can often be chosen by using the biochemical literature to identify a concentration that has an effect in a living cell or in an animal or human. Similarly it is an advantage to identify a concentration of a toxicant that has an effect in a living cell or organism. That dose, or a multiple of that dose, can than be used in the assay panel. For purposes of obtaining physiologically-relevant results it is an advantage to use physiologically-relevant concentrations of reference compounds. Usually these concentrations are in the high-nanomolar to low-micromolar range depending on the potency of the compound.

The invention can be used to identify those compounds with more desirable properties as compared with those compounds with less desirable properties. For example, the invention can be used to establish profiles or ‘fingerprints’ of activity for known toxicants. A compound of pharmacological interest can then be profiled using the same assay panel as for the known toxicant, and the profile compared to that of the known toxicant to determine if there is a similar pattern indicative of potential toxicity. In addition, pharmacological profiles or fingerprints can be established for drugs that are known to be safe and effective; and those fingerprints can be used as guidelines for the development of novel compounds with similar profiles. By comparing pharmacological profiles of a test compound with the pharmacological profiles of reference compounds, unintended and/or undesirable properties of a test compound can be identified. Therefore the present invention is suitable for use in the discovery and development of compounds with desired therapeutic profiles and without undesired adverse or toxic profiles. Those test compounds with the most desirable profiles can then be selected for further development. If this process is applied in an iterative process, such as during the lead optimization phase of drug discovery, leads can be gradually improved in order to achieve a desired pharmacological profile in the cell type that is studied.

The time-course of cellular activity of a compound can be established using this approach. That is, cells (tissues, animals or model organisms) can be treated with a compound of interest for minutes, hours or days, and both the short-term effects and the longer-term effects can be assessed. By short-term effects we mean effects occurring within minutes to hours; by long-term effects we mean effects occurring within hours to days. The approach allows elucidation of the immediate, dynamic consequences of drug action on the pathways of living cells; along with the secondary effects of drugs. Secondary effects may result from changes in the cell cycle, protein synthesis or degradation, gene expression, and other effects that are secondary to the direct action of the drug on its direct target(s). We often test novel compounds at multiple time points in order to capture both short term and long term effects—for example, for short times such as 20 minutes to capture the immediate effects of the compound on pathway flux; and for long times such as 16 hours, to detect effects on events such as DNA damage response pathways and the cell cycle.

Cellular potency of any compound can be determined, and detailed comparisons can be made—for example, between synthetic compounds within a lead series—in order to identify compounds with desired and/or optimal properties including potency, allowing comparisons between leads within a series. Dose-response curves can be established for each compound of interest by contacting the cells in the panel with successively increasing doses of compound. It is not necessary to use massive concentrations of a test compound to see an effect. Effects of pharmacologically active compounds can be observed at concentrations at or near the cellular IC50 of the compound as established in functional assays. For first-pass screening of test compounds we often start with a concentration three times the cellular IC50 and then perform a dose-response curve for those signaling nodes (proteins) that are affected by the test compound.

The present invention is not limited to the cell type used for the assay panels. The cell type can be a human cell, a mammalian cell (mouse, monkey, hamster, rat, rabbit or other species), a plant protoplast, yeast, fungus, or any other cell type of interest. The cell can also be a cell line or a primary cell. In a preferred embodiment of the invention for drug discovery, human cells are used; in an alternative embodiment, mammalian cells are used. The cell can be a component of an intact tissue or animal, or in the whole body; or can be isolated from a biological sample or organ. Any cell of interest can be used, including primary cells and cell lines of any type (epithelial, stromal, hematopoietic, etc.) and any origin (hepatic, cardiac, neural, etc.) The present invention could also be used in fungal cells to identify antifungal agents that block key pathways or in bacterial cells to identify antibiotics or in biowarfare agents to identify antidotes. The present invention can be used in intact cells or tissues in any milieu, context or system. This includes cells in culture, cells ex vivo, organs in culture, and in live organisms. For example, this invention can be used in model organisms such as Drosophila, C. Elegans or zebrafish. This invention can be used in preclinical studies, for example in mice. Mice can be treated with a drug and then a variety of cells or tissues can be harvested and the harvested cells can be used to measure the parameters of interest. This invention can also be used in nude mice, for example, human cells can be implanted as xenografts in nude mice, and a drug or other compound administered to the mouse. Cells can then be rescued from the implant, or the entire implant can be recovered, and the measurements made in the rescued cells or whole implant or tissue. The invention can be used in transgenic animals or organisms in which reporters of interest have been transgenically engineered to report out pathway activity.

The present invention can be used in conjunction with drug discovery for any disease of interest including cancer, diabetes, obesity, cardiovascular disease, inflammation, neurodegenerative diseases, and many other chronic or acute diseases afflicting mankind. Moreover, the invention is not limited to human drug discovery but can be used in the cosmetics or nutraceutical industries, agriculture, food science and/or animal health. For example the invention can be used in cells derived from higher plants to identify chemical agents that stimulate growth-related pathways or that block disease pathways or infections. The invention can be used in the cosmetics industry, for example in keratinocoytes or other cells, as a surrogate for animal testing of new formulations. The invention can be used in cells from food crops (corn, rice, potato, wheat) to identify agents that promote disease resistance, cold hardiness or other acquired or inducible traits.

Choice of Instrumentation and Detection Modes

With the aid of fluorescence technologies and cell imaging instrumentation and the principles and methods of the present invention, it is possible to construct an assay for a large number of dynamic states and transitions in intact cells. Many examples of these and methods for their construction, performance and detection are provided herein using a variety of techniques, reagents and instrumentation which are well known to those skilled in the art. It is an advantage from the standpoint of cost and efficiency to choose from methods and assays that can be automated and that can be performed with standard off-the-shelf robotics and instrumentation; although alternative, manual methodologies can also be employed for lower-throughput applications. Therefore we have focused in particular on methods that can be performed in conjunction with high-throughput instrumentation in intact cells, while recognizing that lysed cells can also be used with this invention.

The choices of assay formats, reagents and detection modes are often dictated by the biology of the process, pathway, states and transitions that are selected for analysis. On the other hand, it is entirely possible to carry out the invention with a single type of instrument system if the measurable states and transitions, and the corresponding assays, are selected to be compatible with the chosen instrumentation. Preferred embodiments involve the generation of fluorescent or luminescent signals that are easily detected in living cells and which can be quantified with any one of a variety of high-throughput instrumentation systems. Preferred embodiments of the invention involve the detection of signals with fluorescence or luminescence spectroscopy, flow cytometry, automated fluorescence microscopes, and cell-based imaging systems. Alternative embodiments include the use of near-infrared dyes. Each type of instrument produces different measurement artifacts and makes different demands on the fluorescent probe. For example, although photobleaching is often a significant problem in fluorescence microscopy, it is not a major impediment in flow cytometry because the dwell time of individual cells in the excitation beam is short. Fluorescence instruments are of four primary types, each providing distinctly different information:
Spectrofluorometers and microplate readers measure the average properties of bulk (μL to mL) samples.
Fluorescence microscopes resolve fluorescence as a function of spatial coordinates in two or three dimensions for microscopic objects (less than −0.1 mm diameter).
Fluorescence scanners, including microarray readers, resolve fluorescence as a function of spatial coordinates in two dimensions for macroscopic objects such as arrays.
Flow cytometers measure fluorescence per cell in a flowing stream, allowing subpopulations within a large sample to be identified and quantified.

Preferred embodiments of the invention utilize fluorescence microscopy and flow cytometry for signal detection. In principle, one type of fluorescence instrument will be better than any other instrument for any particular assay. New assays would ideally be evaluated with several instrumentation types to determine which instrument provides the best reproducibility, reliability, dynamic range, throughput, and signal relative to background.

In matching instrumentation to any particular molecular parameter or assay type, a key question is whether a high-content analysis is required or whether a high-throughput analysis is sufficient. Assays that are utilized in conjunction with fluorescence microscopy are often referred to as high-content assays due to the spatial resolution that can be achieved on the level of an individual cell. The need for high-content analysis is determined by the behavior of the molecular parameter that is to be measured; for example, measurement of the abundance of nuclear protein-protein complexes requires the ability to delineate the cell nucleus. In order to measure an increase or decrease in a state within a particular subcellular compartment, cells are imaged by fluorescence microscopy or confocal imaging and the sub-cellular location of the signal is detected and quantified; usually by co-staining one or more subcellular compartments with a dye or other compartment-specific label and using the signal from that label to define the compartment of interest. Numerous instrumentation systems have been developed to automate cell-based, high-content assays including those sold by Cellomics Inc., Amersham (GE Medical Systems), Q3DM (Beckman Coulter), Evotec GmbH, Universal Imaging (Molecular Devices), Atto (Becton Dickinson) and Zeiss. Proprietary and non-proprietary algorithms suitable for conversion of fluorescence pixel intensity to subcellular location have been described (Cellomics and Biolmage); such image analysis software is often sold in conjunction with the commercially available instrumentation systems. High-content instrumentation can be used to detect using protein tagging approaches; immunofluorescence of endogenous states; and interaction assays, including protein-fragment and enzyme-fragment complementation assays.

Fluorescence assays often use different fluorescent dyes to label different cellular molecules or structures, such as membrane antigens, DNA, intracellular proteins, ions, or organelles. With a flow cytometer, cells flow through one or more lasers, scattering light and perhaps emitting fluorescence. Light scatter provides some information regarding the morphology of the particles—large or small, granular or smooth, for example. The power of flow cytometry arises from the flexibility and sensitivity of fluorescence technology combined with its high speed (1000 cells/second or more) and ability to correlate quantitative data from many simultaneous measurements on each cell. Modern flow cytometry systems, such as the FACStar (Becton Dickinson) or the Agilent 2100 Bioanalyzer for on-chip flow cytometry, are particularly well suited to these analyses. Any of the commercial instruments permit cell-based analyses with multiwell formats and throughput suitable for large-scale drug discovery.

Amnis Corporation has created an imaging flow cytometer called ImageStream for the multispectral imaging of cells in flow. ImageStream generates up to six simultaneous images of each cell in brightfield, darkfield, and multiple colors of fluorescence. The ImageStream is equipped with a 488 nm laser for sensitive fluorescence imaging at rates of up to hundreds of cells per second. These capabilities enable multiparametric cellular assays. Applications include immunofluorescence, quantification of translocation events, and fluorescence in-situ hybridization (FISH).

Several FRET microscopy techniques are available, each with advantages and disadvantages. Wide-field microscopy is the simplest and most widely used technique. FRET is typically measured as the ratio of acceptor emission to donor emission on excitation of the donor, giving a value that is proportional to the degree of physical association between the 2 fluorophores. One of the major drawbacks of wide-field microscopy is the generation of out-of-focus signal. This can be a problem in cases in which relatively thick samples are inspected and when the goal of the experiment is to study molecular events that take place in restricted volumes within the cell. Laser scanning confocal microscopy solves this problem and, by collecting serial optical sections from thick specimens, allows resolving FRET signals in 3 dimensions. A major limitation of confocal microscopy is the availability of standard laser lines of defined wavelength that normally do not allow one to resolve FRET. A recent technological advance, however, has introduced multiphoton confocal microscopy that, by using a tunable laser in the 700- to 1000-nm range, allows the excitation of a wide variety of fluorophores with higher axial resolution, greater sample penetration, limited photobleaching of the chromophore, and reduced damage of the sample.

The intensity-based FRET techniques described above suffer from contamination of the FRET images with unwanted bleed-through components because of the incomplete separation of the donor and acceptor excitation and emission spectra. When using CFPNYFP, for example, excitation of CFP is associated with partial direct excitation of YFP, which therefore will emit independently of FRET. Even more important is the bleedthrough of CFP emission in the YFP channel, which can contribute to >50% of the FRET image. The degree of crosstalk between fluorophores must be assessed for each individual imaging system, and careful choice of filter sets can minimize bleedthrough. Moreover, once the degree of crosstalk has been measured, it can be accounted for in the offline image-processing phase. Recently, a new algorithm has been developed that removes both the donor and acceptor bleedthrough signals and corrects the variation in fluorophore expression level, generating a true FRET signal.

In a recent technical advance, by applying a spectral deconvolution approach, it has been possible to excite simultaneously several GFPs and record, pixel-by-pixel, the emission spectrum from each of them through a 32-channel spectrophotometer. By subsequent mathematical modeling, it has been possible to determine the contribution of each fluorophore to each pixel, and separation of the signal of FITC from the nearly identical signal of GFP has been reported.²⁸ Fluorophore crosstalk is a particularly serious problem when looking at steady-State, intermolecular FRET. In this situation, the intracellular molar ratio between donor and acceptor is difficult to control, and different concentrations of the 2 fluorophores may be misinterpreted as FRET. Such a problem is completely overcome if the intermolecular FRET sensor and the experimental set up allow monitoring of dynamic FRET. In this case, it is possible to establish whether a change in donor to acceptor fluorescence is a true change in FRET by monitoring donor and acceptor fluorescence intensity over time; a true FRET change corresponds to a symmetric change of donor and acceptor fluorescence intensity.

Another approach for imaging steady-state FRET consists in collecting the donor emission before and after photobleaching of the acceptor. If FRET is present, elimination of the acceptor by photodestruction releases the energy transferred from donor to acceptor with consequent brighter emission from the donor. This method is very simple and can be used in any laboratory equipped with a simple commercial fluorescence microscope. However, the correct interpretation of the results obtained is not always straightforward, especially if FRET efficiency is low.^{29,30} An alternative method consists of measuring FRET via donor photobleaching. This technique exploits the fact that photobleaching is proportional to the excited-State lifetime of the fluorophore. Because FRET reduces the lifetime of the donor's excited State, its photobleaching rate decreases proportionally.

Apart from the intensity-based methods described above, more sophisticated technologies for measuring FRET are also available. Fluorescence lifetime imaging microscopy (FLIM) takes advantage of the fact that FRET results in a shortening of the donor's lifetime; by subtracting the fluorescence lifetime of the donor alone from the lifetime of the donor in the presence of the acceptor, the efficiency of FRET can be measured. Another technique is fluorescence correlation spectroscopy (FCS), in which spontaneous fluorescence intensity fluctuations are measured in a microscopic volume and energy transfer efficiency of freely diffusing single molecules can be accurately measured.

Micro- and nano-technologies can be applied to the present invention. For example, Kasili et al. (J. Am. Chem. Soc. 2004 Mar. 10; 126(9):2799-806) have developed a nanobiosensor—a tiny fiber optic probe that has been drawn to a tip of only 40 nanometers (nm) across, which is small enough to be inserted into a cell. Immobilized at the nanotip is a molecule, such as an antibody, DNA or enzyme that can bind to target molecules of interest inside the cell. Because the 40-nm diameter of the fiber-optic probe is much narrower than the 400-nm wavelength of light, only target molecules bound to the bioreceptors at the tip are exposed to and excited by the evanescent field of a laser signal. This group recently performed measurements to investigate the application and utility of nanosensors for monitoring the onset of the mitochondrial pathway of apoptosis in a single living cell by detecting enzymatic activities of caspase-9. The nanosensors used a probe based on a caspase-9 specific substrate, tetrapeptide Leucine-GlutamicAcid-Histidine-AsparticAcid (LEHD), bound to a fluorescent molecule 7-amino-4-methyl coumarin (AMC). The LEHD-AMC covalently attached on the nanoprobe tip of the sensor was cleaved during apoptosis by caspase-9 generating free AMC. An evanescent field was used to excite cleaved AMC and the resulting fluorescence signal was detected. By quantitatively monitoring the changes in fluorescence signals, caspase-9 activity within a single living MCF-7 cell was detected. Additional assays can be constructed for other enzymes and intracellular antigens using a variety of enzyme substrates or antibodies, respectively. In this manner, these nanodevices could be used in conjunction with pharmacological profiling according to the present invention.

Near-infrared (IR) fluorophores (670-1100 nm) have a distinct advantage over visible dyes, in that very low background fluorescence at longer wavelengths provides an excellent signal-to-noise ratio. Furthermore, antibodies labeled with IR dyes at different wavelengths can be used for detection of multiple targets on membranes and in plates, a feature that cannot be accomplished by other technology such as electrochemiluminescence. LI-COR has developed an IR imaging system designed to image membranes and plates for protein application. The imager simultaneously detects two distinct wavelengths. A scanning optical assembly carries two laser diodes that generate excitation light at 680 and 780 nm, as well as two avalanche photodiodes, which detect emitted fluorescence at 720 and 820 nm. Two-color infrared fluorescent technology has been used for the analysis of signal transduction events by in vitro and in situ assays.

Fluorescence recovery after photobleaching (FRAP) and time lapse fluorescence microscopy may also be used in conjunction with the invention. NMR spectroscopy can also be used in conjunction with the measurement of suitable parameters such as allosteric changes of tagged proteins.

The methods and assays provided herein may be performed in multiwell formats, in microtiter plates, in multispot formats, or in arrays, allowing flexibility in assay formatting and miniaturization. Any of these methods can be applied in conjunction with the principles and methods of the present invention and can be combined in any number of ways. For example, a single well of a microtiter well plate can be dedicated to the measurement of (a) an individual parameter of an individual protein; (b) multiple parameters of an individual protein; (c) a single parameter of multiple proteins; or (d) multiple parameters of multiple proteins. Finally, it is possible to automate the entire process of assay construction, including cell plating and feeding, transfection (where necessary), drug or compound addition, fixation and co-staining (where used), sampling and detection. Most of the currently available instruments can be used in conjunction with automated cell culture systems, cell hotels, automated pipettors, and robotic handling systems.

Chemical transfection methods, electroporation, retroviral or adenoviral transfection and reverse transfection methods and arrays (ref. Akceli) can be used to introduce expression vector constructs. These methods are well known to those skilled in the art. Suitable expression vectors must of course be used and the choice of vectors, and vector elements such as promoters, will depend upon the cell of interest. The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes 1 and 11 (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Virtually any dynamic, cell-based assays can be adapted to the present invention. Most of these rely upon fluorescent probes; fluorescent proteins; reconstitution or production of a fluorescent, luminescent or enzymatic signal; immunocytochemical methods; and/or quantum dots. Preferred methods are those not requiring a washing step prior to detection. Preferred embodiments of the invention include methods that can be performed in multiwell or array formats, and instruments that allow automated processing and reading of the results. General methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Co. Inc. (1978), pp. 296-361. Some characteristics of fluorochromes useful for flow cytometry or fluorescence microscopy are shown in the table below; any of these are compatible with the present invention.

| | | | | |
|---|---|---|---|---|
| Probe | Ex (nm) | Em (nm) | MW | Notes |

Reactive and conjugated probes

| | | | | |
|---|---|---|---|---|
| Hydroxycoumarin | 325 | 386 | 331 | Succinimidyl ester |
| Aminocoumarin | 350 | 445 | 330 | Succinimidyl ester |
| Methoxycoumarin | 360 | 410 | 317 | Succinimidyl ester |
| Cascade Blue | 375; 400 | 423 | 596 | Hydrazide |
| Lucifer yellow | 425 | 528 | | |
| NBD | 466 | 539 | 294 | NBD-X |
| R-Phycoerythrin (PE) | 480; 565 | 578 | 240k | |
| PE-Cy5 conjugates | 480; 565; 650 | 670 | | aka Cychrome, R670, Tri-Color, Quantum Red |
| PE-Cy7 conjugates | 480; 565; 743 | 767 | | |
| APC-Cy7 conjugates | 650; 755 | 767 | | PharRed |
| Red 613 | 480; 565 | 613 | | PE-Texas Red |
| Fluorescein | 495 | 519 | 389 | FITC; pH sensitive |
| FluorX | 494 | 520 | 587 | (AP Biotech) |
| BODIPY-FL | 503 | 512 | | |
| TRITC | 547 | 572 | 444 | TRITC |
| X-Rhodamine | 570 | 576 | 548 | XRITC |
| Lissamine Rhodamine B | 570 | 590 | | |
| PerCP | 490 | 675 | | Peridinin chlorphyll protein |
| Texas Red | 589 | 615 | 625 | Sulfonyl chloride |
| Allophycocyanin (APC) | 650 | 660 | 104k | |
| TruRed | 490, 675 | 695 | | PerCP-Cy5.5 conjugate |
| Alexa Fluor 350 | 346 | 445 | 410 | (Molecular Probes) |
| Alexa Fluor 430 | 430 | 545 | 701 | (Molecular Probes) |
| Alexa Fluor 488 | 494 | 517 | 643 | (Molecular Probes) |
| Alexa Fluor 532 | 530 | 555 | 724 | (Molecular Probes) |
| Alexa Fluor 546 | 556 | 573 | 1079 | (Molecular Probes) |
| Alexa Fluor 555 | 556 | 573 | 1250 | (Molecular Probes) |
| Alexa Fluor 568 | 578 | 603 | 792 | (Molecular Probes) |
| Alexa Fluor 594 | 590 | 617 | 820 | (Molecular Probes) |
| Alexa Fluor 633 | 621 | 639 | 1200 | (Molecular Probes) |
| Alexa Fluor 647 | 650 | 668 | 1250 | (Molecular Probes) |
| Alexa Fluor 660 | 663 | 690 | 1100 | (Molecular Probes) |
| Alexa Fluor 680 | 679 | 702 | 1150 | (Molecular Probes) |
| Alexa Fluor 700 | 696 | 719 | | (Molecular Probes) |
| Alexa Fluor 750 | 752 | 779 | | (Molecular Probes) |
| Cy2 | 489 | 506 | 714 | (AP Biotech) |
| Cy3 | (512); 550 | 570; (615) | 767 | (AP Biotech) |
| Cy3.5 | 581 | 596; (640) | 1102 | (AP Biotech) |
| Cy5 | (625); 650 | 670 | 792 | (AP Biotech) |
| Cy5.5 | 675 | 694 | 1128 | (AP Biotech) |
| Cy7 | 743 | 767 | 818 | (AP Biotech) |

Nucleic acid probes

| | | | | |
|---|---|---|---|---|
| Hoechst 33342 | 343 | 483 | 616 | AT-selective |
| DAPI | 345 | 455 | | AT-selective |
| Hoechst 33258 | 345 | 478 | 624 | AT-selective |
| SYTOX Blue | 431 | 480 | ˜400 | DNA |
| Chromomycin A3 | 445 | 575 | | CG-selective |
| Mithramycin | 445 | 575 | | |
| YOYO-1 | 491 | 509 | 1271 | |
| SYTOX Green | 504 | 523 | ˜600 | DNA |
| SYTOX Orange | 547 | 570 | ˜500 | DNA |
| Ethidium Bromide | 493 | 620 | 394 | |
| 7-AAD | 546 | 647 | | 7-aminoactinomycin D, CG-selective |
| Acridine Orange | 503 | 530/640 | | DNA/RNA |
| TOTO-1, TO-PRO-1 | 509 | 533 | | Vital stain, TOTO: Cyanine Dimer |
| | | | | TO-PRO: Cyanine Monomer |
| Thiazole Orange | 510 | 530 | | |
| Propidium Iodide (PI) | 536 | 617 | 668.4 | |
| TOTO-3, TO-PRO-3 | [symbol] | [symbol] | | |
| LDS 751 | 543; 590 | [symbol] | 472 | DNA (543ex/712em), RNA (590ex/607em) |

Cell function probes

| | | | | |
|---|---|---|---|---|
| Indo-1 | 361/330 | 490/405 | 1010 | AM ester. Low/High Ca^{++,} |
| Fluo-3 | 506 | 526 | 855 | AM ester. pH > 6 |
| DCFH | 505 | 535 | 529 | 2′7′Dichorodihydrofluorescein, oxidized form |
| DHR | 505 | 534 | 346 | Dihydrorhodamine 123, oxidized form, |
| | | | | light catalyzes oxidation |
| SNARF | 548/579 | 587/635 | | pH 6/9 |

Fluorescent Proteins

| | | | |
|---|---|---|---|
| Y66F | 360 | 508 | |
| Y66H | 360 | 442 | |
| EBFP | 380 | 440 | (Clontech) Quantum yield 0.18 |
| Wild Type | 396, 475 | 508, 503 | |
| GFPuv | 385 | 508 | (Clontech) |
| ECFP | 434 | 477 | (Clontech) Quantum yield 0.40 |
| Y66W | 436 | 485 | |
| S65A | 471 | 504 | |
| S65C | 479 | 507 | |
| S65L | 484 | 510 | |
| S65T | 488 | 511 | |
| EGFP | 489 | 508 | (Clontech) Quantum yield 0.60 |
| EYFP | 514 | 527 | (Clontech) Quantum yield 0.61 |
| DsRed | 558 | 583 | (Clontech) Quantum yield 0.29 |

Other probes

| | | | | |
|---|---|---|---|---|
| Monochlorobimane | 380 | 461 | 226 | Glutathione probe |
| Calcein | 496 | 517 | 623 | pH > 5 |

Quantum dots (Qdots) are becoming increasingly useful in a growing list of applications including immunohistochemistry, flow cytometry, and plate-based assays, and may therefore be used in conjunction with this invention. Qdot nanocrystals have unique optical properties including an extremely bright signal for sensitivity and quantitation; high photostability for imaging and analysis. A single excitation source is needed, and a growing range of conjugates makes them useful in a wide range of cell-based applications. Qdot Bioconjugates are characterized by quantum yields comparable to the brightest traditional dyes available. Additionally, these quantum dot-based fluorophores absorb 10-1000 times more light than traditional dyes. The emission from the underlying Qdot quantum dots is narrow and symmetric which means overlap with other colors is minimized, resulting in minimal bleed through into adjacent detection channels and attenuated crosstalk, in spite of the fact that many more colors can be used simultaneously. Since each bioconjugate color is based upon the same underlying material (they differ only in size), the conjugation and use methods for one color are easily extrapolated to all of the different colors. Standard fluorescence microscopes are an inexpensive tool for the detection of Qdot Bioconjugates. Since Qdot conjugates are virtually photo-stable, time can be taken with the microscope to find regions of interest and adequately focus on the samples. Qdot conjugates are useful any time bright photo-stable emission is required and are particularly useful in multicolor applications where only one excitation source/filter is available and minimal crosstalk among the colors is required. Quantum dots have been used as conjugates of Streptavidin and IgG to label cell surface markers and nuclear antigens and to stain microtubules and actin (Wu, X., Liu, H., Liu, J., Haley, K. N., Treadway, J. A, Larson, J. P., Ge, N., Peale, F., and Bruchez, M. P. (2003) Immunofluorescent labeling of cancer marker Her2 and other cellular targets with semiconductor quantum dots. Nature Biotech. 21, 41-46). When conjugated to small molecules such as serotonin, direct interaction with native receptors can be characterized (Rosenthal, S. J., Tomlinson, I., Adkins, E. M., Schroeter, S., Adams, S., Swafford, L., McBride, J., Wang, Y., DeFelice, L. J., and Blakely, R. D. (2002) Targeting Cell Surface Receptors with Ligand-Conjugated Nanocrystals. J. Amer. Chem. Soc. 124, 4586-4594). Oligonucleotide conjugates have been used to demonstrate in situ hybridization assays (Pathak, S., Choi, S., Arnheim, N., and Thompson, M. E. (2001) Hydroxylated Quantum Dots as Luminescent Probes for in Situ Hybridization. J. Amer. Chem. Soc. 123, 4103-4104). Peptide conjugates have also been used to enable in vivo studies in mice (Akerman, M. E., Chan, W. C. W., Laakkonen, P., Bhatia, S, N., and Ruoslahti, E. (2002) Nanocrystal targeting in vivo. Proc. Nat Acad. Sci. 99, 12617-12621).

Molecular States and their Measurement in Intact Cells

Here we describe the types of molecular parameters that can be measured and provide examples of the assays suitable for their measurement in intact cells. It is not possible to list all the possible parameters that can be used with the invention, or all of the possible methods for their measurement in intact cells. One skilled in the art will be able to select the individual molecules from the resources provided above and from the scientific literature; use the principles and methods provided herein to design panels of assays for the performance of pharmacological profiling; and evaluate the utility of the panels through empirical testing.

As used herein, suitable states include macromolecules; small molecules; complexes; and the quantity, subcellular compartment(s), and products (of any transitions) of any of the foregoing. States often have the dimensions of space (within compartments of the cell) and time (of the effect that is measured). That is, any state can be measured at any point in time after treatment of a cell with a compound of interest. States can also be measured under various cellular environments or additional treatments, for example, in the absence and presence of a pathway agonist that boosts the signal through a particular pathway. This is shown in Example 2 of this invention, wherein a GPCR-related signaling node was probed in the absence and presence of isoproterenol, a known GPCR agonist.

Pathways, such as the pathways listed in Table 2, contain different macromolecules, each of which has its own identity. As used herein, the term ‘macromolecules’ includes proteins, nucleic acids, lipids, and carbohydrates; and portions, fragments, domains, or epitopes of any of these. Preferred embodiments of molecular parameters include: enzymes, enzyme substrates, products of transitions, antibodies, antigens, membrane proteins, nuclear proteins, cytosolic proteins, mitochondrial proteins, lysosomal proteins, scaffold proteins, lipid rafts, phosphoproteins, glycoproteins, membrane receptors, nuclear receptors, protein tyrosine kinases, protein serine/threonine kinases, phosphatases, proteases, hydrolases, lipases, phospholipases, ligases, calcium-binding proteins, chaperones, DNA binding proteins, RNA binding proteins, scaffold reductases, oxidases, synthases, transcription factors, ion channels, RNA, DNA, RNAse, DNAse, phospholipids, sphingolipids, nuclear receptors, ion channel proteins, nucleotide-binding proteins, proteins, tumor suppressors, cell cycle proteins, and histones. Detailed information about individual proteins can be found in the biochemical literature and in publicly and privately available databases, some of which are provided here. These resources are useful in the selection of network nodes within pathways, and in identifying associated gene sequences and sourcing clones (cDNAs) encoding the proteins in those pathways. The gene identities and related sequences are important for the construction of assays for pharmacological profiling, especially in cases where the assay must be constructed by making and expressing a fusion construct in an expression vector. Since it is difficult, even for one skilled in the art, to keep up with the rapidly increasing number of genomics, proteomics, and interactomics and metabolomics databases, the major sequence and structure repositories are important resources that are listed here (Table 3).

TABLE 3
Major sequence and structure repositories

| Database | Description | URL |
|---|---|---|
| GenBank | Repository of all publicly available annotated | http://www.ncbi.nlm.nih.gov/ |
| | nucleotide and protein sequences | |
| EMBL | Repository of all publicly available annotated | http://www.ebi.ac.uk/embl.html |
| Database | nucleotide and protein sequences | |
| DDBJ (DNA | Repository of all publicly available annotated | http://www.ddbj.nig.ac.jp |
| Data Bank of | nucleotide and protein sequences | |
| Japan) | | |
| PIR | Protein information resource: protein sequence | http://pir.georgetown.edu/ |
| | database | |
| Swiss-Prot | Highly annotated curated protein sequence | http://www.expasy.org/sprot |
| | database | |
| PDB | Protein structure databank: Collection of | http://www.rcsb.org/pdb |
| | publicly available 3D structures of proteins and | |
| | nucleic acids | |

Swiss-Prot is a manually curated protein sequence database with a high level annotation of protein function and protein modifications, including links to property, structure and pathways databases. PIR is similar to Swiss-Prot, with the former providing some options for sequence analysis. Some of the major protein sequence and structure property databases are listed in Table 4. An increasing number of integrated database retrieval and analysis systems tools are being developed for the purpose of data management, acquisition, integration, visualization, sharing and analysis. Table 5 lists examples of these tools. GeneCards is an integrated database of human genes, genomic maps, proteins, and diseases, with software that retrieves, combines, searches, and displays human genome information. GenomNet is of particular interest since its analytical tools are tightly linked with the KEGG pathways database (discussed in the next section). ToolBus comprises several data analysis software platforms such as multiple sequence alignment, phylogenetic trees, generic XML viewer, pathways and microarray analysis, which are linked to each other as well as to major databases. SRS and NCBI serve as general data retrieval portals as well as to provide links to specific analysis tools.

TABLE 4
Protein sequence and structure property databases

| Database | Description | URL |
|---|---|---|
| eMOTIF | Protein sequence motif database | http://motif.stanford.edu/emotif |
| InterPro | Integrated resource of protein families, domains | http://www.ebi.ac.uk/interpro |
| iProClass | Integrated protein classification database | http://pir.georgetown.edu/iproclass/ |
| ProDom | Protein domain families | http://www.toulouse.inra.fr/prodom.html |
| CDD | Conserved domain database: covers protein | http://www.biochem.ucl.ac.uk/bsm/cath/ |
| | domain information from Pfam, SMART and | |
| | COG databases | |
| CATH | Protein structure classification database | http://cl.sdsc.edu/ce.html |
| CE | Repository of 3D Protein structure alignments | |
| SCOP | Structural classification of proteins | http://scop.mrc-lmb.cam.ac.uk/scop |

TABLE 5
Integrated database retrieval and analysis systems

| Database | Description | URL |
|---|---|---|
| GeneCards | Database of human genes, proteins and their | http://bioinfo.weizmann.ac.il/cards |
| | involvement in diseases | |
| GenomeNet | Network of database and computational | http://www.genome.ad.jp/ |
| | services for genome research | |
| NCBI | Retrieval system for searching several linked | http://www.ncbi.nlm.nih.gov |
| | databases | |
| PathPort/ToolBus | Collection of web-services for gene prediction | https://www.vbi.vt.edu/pathport |
| | and multiple sequence alignment, along with | |
| | visualization tools | |
| SRS-EBI | Integration system for both data retrieval and | http://srs.ebi.ac.uk |
| | applications for data analysis | |

Measurements of the Amount of a State

The amount of an individual state within a cell reflects the balance between the rate of synthesis and the rate of degradation of the state at any point in time. For proteins, the processes of protein synthesis and degradation are often influenced by treatments of cells with agents that affect the protein synthetic machinery, the proteasome, and/or the cell cycle. For RNA, the expression of a particular gene is regulated by transcription of DNA and degradation of the resulting RNA. For DNA, the amount of a particular gene or chromosome is affected by the stage of the cell cycle and by processes that result in gene duplication or loss. These events can be assessed in real time and used to report on the activity of compounds on pathways of interest.

The abundance of virtually any endogenous protein can be measured in an intact cell by immunocytochemistry, so long as a sufficiently specific antibody for the measurement of the state of interest is available. By applying antibodies to fixed cells, one can measure the abundance of a particular protein or class of proteins, as well as specific post-translational modifications (e.g. phosphorylation, acetylation, ubiquitination) of a protein or class of proteins or other macromolecules. A preferred embodiment of the current invention uses immunofluorescence assays in human cells in combination with flow cytometry or high-content imaging systems. Many monoclonal and modification-state-specific antibodies are commercially available from commercial suppliers (UpState Biotechnology, Becton Dickinson, Cell Signaling Technologies) or can be generated using standard techniques known to one skilled in the art. We teach that these reagents and methods can be applied to pharmacological profiling in whole cells on a global scale, and provide further examples below. For example, post-translational modifications of proteins can be measured for endogenous proteins in intact cells if a suitably specific antibody is available for the modification. The tyramide signal amplification (TSA) and Enzyme-Labeled Fluorescence (ELF) technologies, are useful for detecting low-abundance targets in cells and tissues. TSA technology also permits far less antibody to be used in the detection scheme, saving the cost of expensive antibodies. The combination of mouse monoclonal antibody labeling with our Horseradish Peroxidase and detection with a fluorescent tyramide yields a very high signal from a very small amount of the primary antibody or a low copy number of the target. Chemiluminescence detection may also be adapted to immunocytochemistry and in situ hybridization protocols.

The invention can also be used in conjunction with phenomenological assays such as those provided by Dunnington et al. (ref). These and other antibodies or targeted probes can be used in conjunction with a wide variety of functional markers, biological dyes or stains, including stains of subcellular compartments (nucleus, membrane, cytosol, mitochondria, golgi, etc.); ion-sensitive dyes such as calcium-sensitive dyes; dyes that measure apoptosis or changes in cell cycle State; DNA intercalating dyes; and other commonly used biochemical and cell biological reagents. For example, co-staining of subcellular compartments would allow the fine details of the effects of drugs to be assessed, as we show below for cells co-stained with a nuclear dye) and/or a membrane stain. Such biochemical reagents and methods for their use are well known to those skilled in the art.

Incorporation of 5-bromo-2′-deoxyuridine (BrdU) into newly synthesized DNA permits indirect detection of rapidly proliferating cells with fluorescent dye-labeled anti-BrdU antibodies, thereby facilitating the identification of cells that have progressed through the S-phase of the cell cycle during the BrdU labeling period. Fluorescent conjugates of monoclonal anti-BrdU antibody labeled with photostable dyes such as Alexa Fluor 488, 532, 546, 594, 647 and 660 dyes are available. This anti-BrdU antibodies are also available as a biotin-XX conjugate in addition to its use for detecting BrdU-labeled DNA, monoclonal PRB-1 recognizes bromouridine (BrU) incorporated into RNA, which provides one of the few methods for specific localization of RNA in cells. It should be possible to amplify the detection of very low degrees of BrdU incorporation by using the biotin-XX conjugate of anti-BrdU in conjunction with streptavidin-based tyramide signal amplification (TSA).

A preferred embodiment of this invention utilizes the expression of tagged (chimeric) proteins. A cell can be transiently transfected with a fusion construct in a suitable expression vector, wherein a gene (such as a human cDNA encoding a protein that represents a signaling node) is fused in frame with a peptide or protein reporter. Given a suitable expression vector and transfection protocol, a chimeric protein is then expressed in the live cell. Different tags allow tracking of the abundance, subcellular location, and/or activity of an expressed protein. Many examples of this are provided below, including tagging with a fluorescent protein such as GFP to monitor activity, interactions, conformation, location, structure or stability of proteins; epitope tagging; and tagging with polypeptide fragments of reporters, such as for protein-fragment complementation and enzyme-fragment complementation assays. Epitope tagging is a versatile strategy for detecting proteins expressed by cloned genes; detection and purification of the epitope-tagged fusion-protein can be mediated by antibodies to the engineered peptide, thus eliminating the need for antibodies to proteins from each newly cloned gene. Fusion proteins may also encompass an antibody fragment to be detected. Anti-tag reagents can be applied to a broad variety of biomolecular interactions.

Homogeneous time-resolved fluorescence (HTRF), as well as most of the other methods used in drug screening, enables the labeling of biological entities for the direct observation of interactions. HTRF anti-tag reagents have been assembled to present a comprehensive toolbox of carefully selected antibodies offering many possibilities for the study of molecular mechanisms. All anti-tag antibodies are available as Europium-cryptate and XL665 conjugates. MAb GSS11 is a mouse IgG2a raised against GST from Schistosoma japonicum. This monoclonal was shown to react with GST-tagged fusion protein from a large number of expressing vectors. 2,4-dinitrophenyl (DNP) is a widely used organic motif for peptide and oligonucleotide tagging. Anti-DNP mouse MAb 265.5 exhibits a high affinity for DNP-derivatized compounds. Peptidic tags are also commonly represented in expression vectors. Corresponding immunodetection tools have been developed for the 6-histidine motif (HIS-1, mouse IgG2a), the c-myc EQKLISEEDL sequence (9E10, mouse IgG1), the FLAG® DYKDDDDK octapeptide (M2, mouse IgG1) and the hemagglutinin YPYDVPDYA motif (HAS01, mouse IgG1). For example, the association of a GST-tagged protein with another 6HIS fusion molecule may be visualized using anti-GST and anti-6HIS HTRF conjugates.

To report out the activity of an intracellular pathway, it is important to express fusion proteins at a low enough level that it does not perturb the normal behavior of the cell; thus, massive overexpression is to be avoided. Usually this can be accomplished by titrating the amount of the DNA construct that is transfected such that the amount of DNA used is just sufficient to allow signal detection. Generation of stable cell lines in which a tagged gene is integrated into the genome usually allows for low-level expression in an entire population of cells and provides a more homogeneous population of cells for analysis. For purposes of reporting the amount of a protein of interest, the tag can be either a peptide tag, such as an epitope tag or native epitope, or a fluorescent protein.

A strong contribution to the development of bioimaging techniques has come from the molecular cloning and subsequent engineering of green fluorescent protein (GFP) from the bioluminescent jellyfish Aequorea voctoria. GFP has several qualities that make it ideal for in vivo imaging. First, GFP can be expressed in a variety of cells, where it becomes spontaneously fluorescent without the need for cofactors. Second, because it is a protein, GFP can be tagged with an appropriate signaling peptide and expressed as such or fused to another protein in specific organelles, such as the mitochondria, the nucleus, or the endoplasmic reticulum. Finally, mutagenesis of GFP has generated many mutants with varying spectral properties, thus allowing imaging of several different fluorescent proteins simultaneously. Due to these properties, GFP has been successfully used as a marker for studying gene expression as well as protein folding, trafficking, and localization. Indeed, GFP-tagged proteins have been developed that can monitor activation of signaling components or generation of second messengers as the process happens within a living cell, allowing the dynamics of such events to be recorded in real time and space. Many of these examples have been published in the literature (Tavare). Fluorescent proteins can be applied to monitoring individual states and transitions in living cells; therefore, such methods can be readily applied to the construction of assay panels for pharmacological profiling. Simple translocation of such detectors can sometimes reflect the buildup of an activated protein or second messenger in a specific compartment. Antibodies against GFP facilitate the detection of native GFP, recombinant GFP and GFP-fusion proteins by immunofluorescence.

The various states of DNA and RNA can also be measured in intact cells using a variety of hybridization techniques. For example, the amount of a particular DNA or DNA region can be measured by fluorescence in situ hybridization which allows quantification of DNA copy number. The amount of a particular mRNA can be measured by hybridization with a sequence-specific probe such as a branched DNA probe or an oligonucleotide probe that is tagged so as to allow for in situ hybridization. Suitable reagents and instrumentation for their use for ISH and FISH are provided by Ventana Medical Systems, Inc. (Tucson, Ariz.); BioGenex, Inc. (San Ramon, Calif.) and by GenoSpectra (Fremont, Calif.) and Vysis, Inc. (Downers Grove, Ill.).

Many if not all macromolecules exist as components of macromolecular complexes which can vary in size, complexity and identity and can respond dynamically to a cell treatment by undergoing transitions. At a minimum, a complex is a binary complex between a first molecule and a second molecule. As a state, a binary complex is the product of a transition, wherein the transition is an interaction or association of two molecules. Thus in principle one could choose to measure either the interaction or association itself (the transition) or the product of the transition (the complex).

The amount of a macromolecular complex within a cell reflects the balance between the rate of synthesis and the rate of degradation of the associated components at any point in time. The processes of protein synthesis and degradation are often influenced by treatments of cells with agents that affect the protein synthetic machinery, the proteasome, and/or the cell cycle. In a binary complex, either the first or second molecules may be a macromolecule or a small molecule. Thus, complexes may form between and among proteins; DNA; RNA; lipids; carbohydrates; and macromolecules and small molecules, such as ligands, hormone, cytokine, or growth factor; a drug or a drug candidates or a lead compound; a natural product; a dye; a synthetic molecule; a toxicant; a metal; and an ion. These events can be assessed in real time and used to report on the activity of pathways of interest.

A variety of assays have been constructed for measurements of macromolecular complexes. Enzyme-fragment complementation assays are based either on activity of wild-type beta-galactosidase or on the phenomenon of alpha- or omega-complementation. Beta-gal is a multimeric enzyme which forms tetramers and octomeric complexes of up to 1 million Daltons. beta-gal subunits undergo self-oligomerization which leads to activity. This naturally-occurring phenomenon has been used to develop a variety of in vitro, homogeneous assays that are the subject of over 30 patents. Alpha- or omega-complementation of beta-gal, which was first reported in 1965, has been utilized to develop assays for the detection of antibody-antigen, drug-protein, protein-protein, and other bio-molecular interactions. The background activity due to self-oligomerization has been overcome in part by the development of low-affinity, mutant subunits with a diminished or negligible ability to complement naturally, enabling various assays including for example the detection of ligand-dependent activation of the EGF receptor in live cells.

Protein-fragment complementation assays (PCA) represent a particularly useful method for quantifying of the amount and subcellular locations of macromolecular complexes within a cell, in particular for protein-protein, protein-RNA and protein-DNA complexes. With PCA, proteins are expressed as fusions to engineered polypeptide fragments, where the polypeptide fragments themselves (a) are not fluorescent or luminescent moieties; (b) are not naturally-occurring; and (c) are generated by fragmentation of a reporter. Michnick et al. (U.S. Pat. No. 6,270,964) taught that any reporter protein of interest can be used for PCA, including any of the reporters described above. The ability to choose among a wide range of reporter fragments enables the construction of fluorescent, luminescent, phosphorescent, or otherwise detectable signals; and the choice of high-content or high-throughput assay formats. Thus, reporters suitable for PCA include, but are not limited to, any of a number of enzymes and fluorescent, luminescent, or phosphorescent proteins. Small monomeric proteins are preferred for PCA, including monomeric enzymes and monomeric fluorescent proteins, resulting in small (−150 amino acid) fragments. Most preferably, PCAs for the present invention are constructed using a fluorescent protein such as a YFP or Venus variant of GFP; a luciferase, such as Gaussia, renilla or firefly luciferase; a beta-lactamase or beta-glucuronidase; or a dihydrofolate reductase. Since any reporter protein can be fragmented using the principles established by Michnick et al., assays can be tailored to the particular demands of the cell type, target, signaling process, and instrumentation of choice. Protein-protein, protein-RNA and protein-DNA complexes can all be probed using PCA. As we have shown previously and in the present invention, the fragments engineered for PCA are not individually fluorescent or luminescent. This feature of PCA distinguishes it from other inventions that involve tagging proteins with fluorescent molecules or luminophores, such as U.S. Pat. No. 6,518,021 (Thastrup et al.) in which proteins are tagged with GFP or other luminophores. A PCA fragment is not a luminophore and does not enable monitoring of the redistribution of an individual protein. In contrast, what is measured with PCA is the formation of a complex between two proteins. Finally, PCAs can be used in conjunction with a variety of existing, automated systems for drug discovery, including existing high-content instrumentation and software such as that described in U.S. Pat. No. 5,989,835.

Whether a state is an individual molecule or a complex between molecules, it may have a preference for a particular subcellular compartment in a cell. In addition, treatment of a cell with a compound may lead to transportation of the state(s) from one compartment to another, or to an increase or decrease in the amount of a particular state within a compartment as a result of synthesis or degradation. Any of these transitions may alter the subcellular distribution of the state(s). Subcellular compartments vary by cell type, in particular, whether the cell is from a eukaryote or a prokaryote. For mammalian (eukaryotic) cells, various subcellular compartments include the cytosol; nucleus; membrane (plasma membrane and nuclear membrane); mitochondria; Golgi; lysosome; endosome; and endoplasmic reticulum. The subcellular compartment of a state can be assessed using ‘high-content’ imaging systems that provide information on the subcellular distribution of a fluorescence signal. Since transportation is a transition, measurement of a change in subcellular distribution of a state provides a measurement of the occurrence of a transition.

In addition to macromolecules, states that may be used in pharmacological profiling include small molecules. As used herein, the term ‘small molecule’ includes chemical compounds; biologic compounds; synthetic molecules; drugs; toxicants; lead compounds; natural products; nucleotides or polynucleotides; peptides; ligands; metabolites; second messengers; dyes; ubiquitin or a ubiquitin-like molecule; small interfering RNAs; probes; fluorophores; and quantum dots. Chemical and biologic compounds include small molecules that are substrates or products of reactions. Second messengers are small molecules that transmit information and influence the behavior and activity of other molecules; these include cyclic nucleotides (cAMP), inositol phosphates (IP3), calcium, and other molecules and ions that are released and/or secreted in response to cell stimuli.

The amounts of these can be measured with a wide variety of cell-based assays. Virtually any of these molecules can be derivatized with a fluorophore such that its uptake, transportation, and subcellular location within cells can be tracked. Also, molecular engineering of GFP has enabled the generation of active sensors capable of monitoring complex processes, such as intracellular second messenger dynamics and enzyme activation. The generation of GFP mutants with distinct excitation and emission spectra, as well as the molecular cloning of new fluorescent proteins from coelenterate marine organisms, has provided several fluorophores that can serve as donor/acceptor pairs for fluorescence resonance energy transfer (FRET). FRET relies on a nonradiative, distance-dependent transfer of energy from a donor fluorophore to an acceptor fluorophore. For FRET to occur, the donor-acceptor distance must be between 2 and 6 nm, the 2 fluorophores must be appropriately oriented in space, and there must be a substantial overlap (>30%) between the donor's emission spectrum and the acceptor's excitation spectrum. In FRET, the donor is excited by incident light, and, if an acceptor is in close proximity, the excited State energy from the donor can be transferred. This leads to a reduction in the donor's fluorescence intensity and excited lifetime and an increase in the acceptor's emission intensity. At present, the best pair for FRET consists of the cyan and yellow mutants, CFP and YFP. CFP is much brighter and more photostable then BFP. The first YFP mutants showed a marked sensitivity to H⁺ and Cl⁻ ions. These properties, although successfully exploited for directly measuring intracellular pH and Cl⁻ concentration, represent a source of artifacts in some FRET applications. Therefore, YFP has been additionally engineered to generate a new variant (citrine) that overcomes these problems and furthermore shows greater photostability. Another mutant of YFP is Venus, a very bright and fast-maturing variant. Much effort has been placed on the search for more red-shifted fluorescent proteins (RFPs) to be used as FRET acceptors in combination with a GFP donor. RFPs would provide greater tissue penetration and minimize tissue autofluorescence background; however, additional improvement of the existing proteins will be necessary for their useful application in FRET experiments. The major limitation of the original dsRed is that it forms tetramers and therefore can tetramerize any cellular protein to which it is fused. This can lead to large aggregation of the fusions or, if the cell protein is resistant to tetramerization, to lack of fluorescence (our unpublished observation, 2004). By a combination of site-directed and random mutagenesis, a monomeric variant of this RFP has been generated (mRFP-1) in which most of the problems of dsRed have been overcome. However, mRFP-1 performance as a FRET acceptor remains hampered by the very long tail of its excitation spectrum on the short wavelength side, leading to direct excitation of the acceptor when exciting the donor.

The first sensors based on dynamic FRET to be developed were probes for measuring Ca²⁺-CaM or free Ca²⁺ fluctuations. In the latter case, the general design of the sensor consists in the tandem fusion of CFP, CaM, the CaM-binding domain from smooth muscle myosin light chain kinase (M13), and YFP. After an increase of Ca²⁺ concentration, the CaM component binds Ca²⁺ and preferentially wraps around the fused M13 peptide. This conformational change results in a decrease of the distance between the 2 GFP mutants and, therefore, an increase in FRET. These Ca²⁺ sensors, named cameleons, have been subsequently modified and targeted to specific subcellular compartments and have been used to monitor, for example, Ca²⁺ dynamics that occur locally at the secretory vesicle surface, in caveolae, or in the nucleus, a result difficult to obtain with conventional Ca indicators such as Fura-2.

Probes based on dynamic FRET have been developed also for other soluble intracellular second messengers, such as cAMP and cyclic GMP (cGMP). A sensor for cAMP has been generated by genetically fusing the catalytic (C) subunit of PKA to YFP and the regulatory (R) subunit of PKA to CFP. When cAMP is low, the GFP-tagged PKA forms a heterotetramer in which CFP and YFP are close enough for FRET to occur. When cAMP levels rise, YFP-C dissociates from CFP-R and FRET disappears. By using such a sensor, it is possible to demonstrate that cAMP generated via β-adrenergic receptor stimulation does not behave as a freely diffusible second messenger but is compartmentalized.

Transitions and their Measurement in Intact Cells

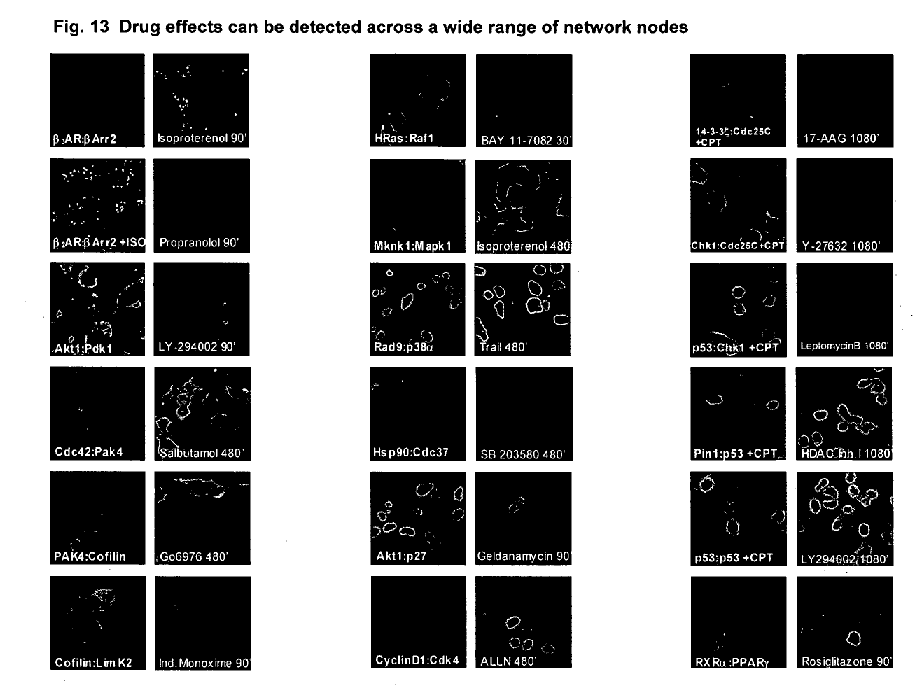

Transitions (FIG. 4) that can be used in conjunction with this invention may include chemical modification; replication; synthesis; degradation; transcription; translation; alternative splicing; transportation; non-covalent modification; cleavage; addition or removal; allosteric change; structural change; redox change; solubility change; association; dissociation; interaction; binding; and multimerization. As transitions, the terms ‘addition’ and ‘removal’ include such processes of chemical modification and/or non-covalent modification; including phosphorylation/dephosphorylation; methylation/demethylation; fatty acylation/deacylation; ubiquitination or SUMOylation; epitope addition or loss; glycosylation/deglycosylation; removal or addition of a heme; nitrosylation/denitrosylation; oxidation/reduction; acetylation/deacetylation; myristylation/demyristylation; prenylation (such as farnesylation)/deprenylation; removal or addition of an amino acid or nucleotide; and binding or loss of another molecule. Examples of drug effects on all of these processes are shown in FIGS. 13-23.

Interactions of molecules that often reflect pathway modulation in living cells. It should be noted that the product of an interaction of two molecules is a complex between the molecules that interact; the complex is a new state that is often associated with a particular subcellular compartment and which can translocate in response to pathway modulators. Thus, a nearly universal way of probing for pathway activity involves detecting and quantifying the amount and/or the subcellular location of a particular complex within a cell following exposure of the cell to a compound of pharmacological interest. Importantly, the effects of a drug on a pathway can be probed by quantifying either the process of interaction (the transition) or the product of an interaction (the complex).

Current thinking regarding macromolecular complexes has been dominated by the use of yeast two-hybrid methods and mass spectroscopy. Although such methods are capable of identifying proteins that bind to each other, they do not allow dynamic studies of networks within cells of interest, such as human cells which are the targets of drug discovery and which contain the cellular machinery relevant for human biology.

Local detection of protein-protein interactions with nanometer resolution is one of the applications of FRET-based biosensors. GFP-based FRET indicators follow two basic designs: unimolecular indicators, in which two protein-interacting domains are sandwiched between CFP and YFP, and bimolecular indicators, in which the fluorophores are fused to two independent domains whose interaction depends on ligand binding or a conformational change of one of the domains. In general, unimolecular sensors may be preferable because a single, unimolecular probe is less likely to interact with bystanding partners. Such interaction may interfere with endogenous reactions and thus affect cell physiology and reduce the probe sensitivity. Unimolecular constructs have the additional advantage of containing equimolar amounts of donor and acceptor fluorophores, therefore allowing maximal exploitation of the dynamic range of the FRET changes and facilitating quantitation. However, several bimolecular FRET-based probes have been generated and used successfully.

FRET imaging has been used to study the association in a macromolecular complex of the multiscaffolding A-kinase anchoring protein AKAP-79, protein kinase A (PKA), and the protein phosphatase calcineurin (CaN) (Reference). Such a multiprotein signaling complex is localized to excitatory neuronal synapses, where it is recruited to glutamate receptors by interaction with membrane-associated guanylate kinase scaffold proteins. This mechanism is thought to play an important role in the modulation of synaptic plasticity. The effects of chemical compounds (drugs, toxicants) on the assembly of multiprotein signaling complexes containing receptors, protein kinases, protein phosphatases, and their substrates would provide compartmentalized readouts of inhibitors of these and similar signal transduction pathways.

In some applications, either the donor or the acceptor fluorophores have been linked to lipids. In this way, FRET measurements have been used to detect either protein interactions with phospholipid bilayers or protein interactions with the plasma membrane.

Measurements of Enzyme Activity in Cells

U.S. Pat. No. 9,469,154 describes methods for the construction of fluorescent protein indicators of protein activity. These methods can be applied to pharmacological profiling according to the present invention. Tsien and Baird created dynamic indicators by inserting various sensor polypeptides into GFP, YFP or CFP. The sensor polypeptide can be designed to measure a variety of parameters related to protein activity, binding, modification, or second messenger release. For example, the sensor polypeptide can be a moiety that undergoes a conformational change upon interaction with a molecule, oxidation-reduction, or changes in electrical or chemical potential. The sensor polypeptide can be a domain of an endogenous protein such as kinases, receptors, ligand-gated channels, voltage-gated channels, protease substrates, enzymes, antigens or antibodies. A change in conformation of the sensor polypeptide in response to stimulus or environment results in a change in fluorescence of the fluorescence indicator. In an in vivo assay, cells transfected with a vector encoding the chimeric sensor protein can be used to assay for the presence of a drug that affects the parameter detected by the particular sensor. These sensors can be constructed for a wide variety of signaling proteins and pathways. Arrays of such sensors can be used for pharmacological profiling according to the present invention or can be combined with other methods specified herein to provide comprehensive pathway coverage.

The activity of kinases can be determined by constructing a fluorescent indicator that responds to increased phosphorylation by an increase or decrease in signal. For example, Nagai et al. (Nature Biotechnology 18: 313-316; 2000) constructed a fluorescent indicator for visualizing cAMP-induced phosphorylation in living cells. The indicator is composed of two green fluorescent protein (GFP) variants joined by the kinase-inducible domain of the transcription factor cyclic adenosine monophosphate (cAMP)-responsive element binding protein (CREB). Phosphorylation of the kinase-inducible domain by the cAMP-dependent protein kinase A (PKA) decreased the fluorescence resonance energy transfer (FRET) between the GFPs. By transfecting cells with an expression vector encoding this indicator protein they were able to visualize activation dynamics of PKA in living cells.

Sato et al. (Nature Biotechnology 20:287-294; 2002) developed genetically encoded fluorescent indicators named ‘phocuses’. Two different color mutants of GFP were joined by a tandem fusion domain composed of a substrate domain for the protein kinase of interest, a flexible linker sequence and a phosphorylation recognition domain that binds with the phosphorylated substrate domain. Intramolecular interaction of the phosphorylated substrate domain and the adjacent phosphorylation recognition domain within a phocus was dependent upon the phosphorylation of the substrate domain by protein kinase, which influenced the efficiency of FRET between the GFPs within a phocus. Similar biosensors have been constructed to examine the activity of intact biologically active proteins (for a review see F. Gaits and K. Hahn 2003: www.stke.orq).

Cardone et al. (US 20030170850) constructed a variety of assays for kinase activity have in live cells. With the approach, a protein which is either a signaling enzyme itself—or its substrate—is tagged in such a way that the signal generated by the tag increases, decreases or is redistributed in response to an agent that regulates a kinase of interest. A label—such as GFP—is associated with the ‘signaling substrate’; alternative labels are enzymatic reporters such as beta-galactosidase, luciferase, alkaline phosphatase, chloramphenicol acetyl transferase, and beta-lactamase, which are capable of producing signals by generating detectable enzymatic products. When a cell is exposed to a compound of interest, changes in the intensity and/or subcellular location of the label are indicative of whether the compound modulates the kinase activity that affects the chosen signaling substrate. Since a detectable (fluorescent or luminescent) reporter is associated with the signaling substrate, the assay can be used to detect the effects of compounds that modulate the activity of the kinase of interest in situ. Examples are provided by Cardone et al. for assays of kinases that regulate discrete proteins in the ubiquitin/proteasome pathway. These assay approaches can be used in conjunction with the present invention by multiplexing these and other assays representing diverse network nodes (pathways) to create an assay panel capable of reporting the activity of a compound on multiple pathways.

Reactive fluorescent dyes can be used in a variety of assays suitable for the present invention; methods for their preparation and use are readily available in the literature and from commercial providers (HiLyte Biosciences, Inc.) Reactive fluorescent dyes are used to modify amino acids, peptides, proteins, antibodies, nucleic acids, and other biological molecules; in particular, amine-reactive dyes have been used to prepare bioconjugates for immunochemistry, fluorescence in situ hybridization, receptor binding and other biological applications. Drugs, ligands and natural toxins can be fluorescently labeled in this manner and can be used to assay cell surface receptors and—if the molecule is membrane permeant—to study dynamic changes in intracellular proteins that lead to changes in binding properties. These dyes can also be adapted to FRET and to immunofluorescence assays for protein quantification and localization (Hung S C et al. 1997; Optimization of spectroscopic and electrophoretic properties of energy transfer primers; Anal Biochem 252: 78-88; Buranda T. et al., 2001, Detection of epitope-tagged proteins in flow cytometry: FRET-based assays on beads with femtomole resolution, Anal Biochem 298: 151-162).

DNA topoisomerases have been assayed with fluorescent probes. Topoisomerases are targeted by a variety of antimicrobial and antineoplastic drugs. In addition to their therapeutic value, these drugs provide tools that can be used to probe the pathways leading to activation/inactivation of DNA topoisomerases. Eukaryotic Type II topoisomerases are blocked by epipodophyllotoxins, acridines and quinolones; topoisomerase I is susceptible to camptothecin. We showed above that camptothecin can be used to stimulate DNA damage response pathways, and that drugs that inhibit the response pathway can be identified by measuring the interactions of proteins in the pathway. Probing for activity of the enzymes in the pathway is an alternative to measuring interactions or post-translational modifications and also provides an example of how enzymatic activity can be used in conjunction with the invention.

The coumarin drugs, novobiocin and coumermycin, are classical inhibitors of DNA gyrase but also inhibit the ATP-dependent eukaryotic type 11 topoisomerases at higher drug concentrations. The coumarin drugs have intrinsic fluorescence; as they bind to topoisomerase, the absorbance and fluorescence of the drugs change as a consequence of interaction with protein (Sekiguchi et al., 1995, mechanism of inhibition of Vaccinia DNA topoisomerase by novobiocin and coumermycin, JBC 271 (4): 2313-2322).

Molenaar (2003) used fluorescent probes which specifically bind to a signaling molecule; using a fluorescent microscope to examine where a fluorescent molecule was at different times, the movement of the structures containing these molecules could be followed. For example, Molenaar followed the tips of chromosomes (telomeres) in three dimensions over the course of time.

The mobility of populations of molecules can also be visualised using FRAP (Fluorescence Recovery After Photobleaching). The fluorescent molecules in a small part of the cell are destroyed when a laser is focused on them. However, although it no longer fluoresces, the biomolecule to which the fluorescent molecule is attached remains intact. The rate at which the fluorescent molecules from the surroundings move into this dark area says something about the mobility of, for example, a certain type of RNA or protein. This mobility in turns provides further information about the functioning of the molecules. One feature of GFP variants, photobleaching, has recently been combined with an older technique known as fluorescence recovery after photobleaching (FRAP) to study protein kinetics in vivo. During photobleaching, fluorochromes get destroyed irreversibly by repeated excitation with an intensive light source. When the photobleaching is applied to a restricted area or structure, recovery of fluorescence will be the result of active or passive diffusion from fluorescent molecules from unbleached surrounding areas. Fluorescence loss in photobleaching (FLIP) is a variant of FRAP where an area is bleached, and loss of fluorescence in surrounding areas is observed. FLIP can be used to study the dynamics of different pools of a protein or can show how a protein diffuses, or is transported through a cell or cellular structure. These photobleaching fluorescent imaging techniques, have been applied to proteins of the MAPK pathway (Van Drogen & Peter, 2004, Revealing protein dynamics by photobleaching techniques, in: Methods Mol Biol 284: 287-306).

Enzyme activities can be measured with synthetic, nonfluorescent enzyme substrates. Flow cytoenzymology is a branch of flow cytometry in which fluorogenic substrates measure enzyme activity within cells. Many different fluorescent tags have been conjugated to substrates and used to measure intracellular enzyme activity, including rhodamine 110, fluorescein, 7-amino-4-methyl coumarin, 4-methoxy-2-napthylamine, and 7-amino-4-trifluoro-methyl coumarin. Currently-available substrates consist of two leaving groups conjugated to a dye molecule. The conjugation of the leaving groups to the dye quenches the dye's fluorescence. When the bonds between the leaving groups and dye are cleaved by the enzyme, the fluorescence is released. Synthetic substrates consist of two leaving group sequences conjugated to a fluorescent moiety, either fluorescein via ester bonds or rhodamine-110 via amide bonds. The choice of leaving group sequence for each reagent was based on reported substrate specificity of the target enzyme. The leaving groups include peptides for proteolytic enzymes, sugar moieties for glycosidases, or acyl groups for esterases. Substrates cross the cell membrane by passive diffusion across the cell membrane or either active or passive transport through channels in the cell membrane or may be introduced by chemical or electroporation techniques or by microinjection. Substrates then bind with high affinity to the active site of the enzyme, then the bond between the dye and the leaving group is cleaved and the enzyme releases the products. For example, aminopeptidases are a family of enzymes that cleave N-terminal amino acids from peptide chains. Di-(Glycyl)-Rho110 is a substrate for aminopeptidases containing a single amino acid leaving group. In addition to the activity of individual proteolytic enzymes, complicated cellular processes can also be measured with fluorogenic substrates. These techniques can be used to measure complex cellular processes with fluorogenic substrates for the purpose of pharmacological profiling.

Post-Translational Modifications as Measurable States

In a canonical signaling pathway, binding of agonists to membrane receptors induces a cascade of intracellular events mediated by interactions of other signaling molecules. These events cause a coordinated cascade of intracellular events that ultimately reaches the nucleus and influences the behavior of the living cell. Often, post-translational modifications of particular proteins or other macromolecules occur dynamically in response to an agonist, an antagonist or an inhibitor of a pathway. Many modifications are well characterized, and researchers now associate them with specific agonists and biological outcomes.

Frequently, such signaling cascades involve cycles of post-translational modifications of proteins, such as phosphorylation and dephosphorylation by kinases and phosphatases, respectively. These events are carried out by distinct protein kinases, which phosphorylate other proteins on serine, threonine or tyrosine residues. In turn, protein phosphatases are responsible for dephosphorylating other proteins. From a network perspective such events involve transitions that start with physical interactions of proteins, such as interactions of kinases with their substrates; and interactions of so-called ‘second messengers’ such as calcium, inositol triphosphate and cyclic AMP with their targets.

Measurements of the amount and/or location of two or more phospho-proteins in the absence and presence of the pathway agonist can be used in pharmacological profiling, where the phospho-proteins serve as sentinels of pathway activity. For example, a drug acting upstream of a sentinel would block or inhibit the phosphorylation of the sentinel in response to a cellular stimulus (see FIG. 2). Thus, the phosphorylation status of the phosphoprotein in the absence or presence of a chemical compound can reveal whether or not the test compound acts on that pathway, thereby providing information on drug selectivity.

States involving a variety of post-translational modifications may be used with this invention. Such post-translational modifications include methylation, nitrosoylation, acetylation, farnesylation, glycosylation, myristylation, ubiquitination, sumoylation, and other modifications. Ubiquitin ligases act upon their substrates to effect ubiquitination; myristoyl transferases act upon their substrates to effect myristoylation; proteases act upon their substrates to effect proteolytic cleavage; etc. Either the transition itself can be measured (the activation or inhibition of the enzyme carrying out the modification) or the new state produced by the transition can be measured (the post-translationally-modified molecule). Such transitions can often be measured in intact cells, for example by binding of a fluorescent probe (ligand, substrate, metabolite, second messenger, peptide, dye or other reagent); by conversion of a substrate to a fluorescent product; or by a change in protein conformation as measured with an optical indicator.

Modification-state-specific antibodies allow for the detection of the net changes in the post-translational modifications that result from activation and inhibition of signal transduction pathwaysm and are particularly useful for the present invention since the methods for their use are well known to those skilled in the art. For example, antibodies can be used to quantify the phosphorylation status of proteins. Such antibodies have become standard reagents in research laboratories, and are used in conjunction with a number of in vitro methods that include Western blotting, immunoprecipitation, ELISA (enzyme-linked immunoabsorbent assays), and multiplexed bead assays. Modification-state-specific antibodies can, in principle, be generated for any macromolecule that undergoes a post-translational modification in the cell. Such alterations may be detected using antibodies in conjunction with immunofluorescence, as described herein; however, the method is not limited to the use of antibodies.

Alternative (non-antibody) probes of target or pathway activity can be used, so long as they (a) bind differentially upon a change in a macromolecule in a cell, such that they reflect a change in pathway activity, cell signaling, or cell state related to the effect of a drug; (b) can be washed out of the cell in the unbound state, so that bound probe can be detected over the unbound probe background; and (c) can be detected either directly or indirectly, e.g. with a fluorescent or luminescent method. A variety of organic molecules, peptides, ligands, natural products, nucleosides and other probes can be detected directly, for example by labeling with a fluorescent or luminescent dye or a quantum dot; or can be detected indirectly, for example, by immunofluorescence with the aid of an antibody that recognizes the probe when it is bound to its target. Such probes could include ligands, native or non-native substrates, competitive binding molecules, peptides, nucleosides, and a variety of other probes that bind differentially to their targets based on post-translational modification states of the targets. It will be appreciated by one skilled in the art that some methods and reporters will be better suited to different situations. Particular reagents, fixing and staining methods may be more or less optimal for different cell types and for different pathways or targets.

Modifications of other proteins provides information on drugs that affect DNA damage and apoptosis. For example phosphorylation of histone H2A.X occurs in response to agents that cause DNA double-strand breaks, including ionizing radiation or agents such as staurosporine or etoposide. Drugs that block the pathways leading to DNA damage cause a decrease in phosphorylation of histone H2A.X. Therefore, assays for histone H2A.X can be used in pharmacological profiling to identify and compare agents that block or induce the pathway leading to H2A.X phosphorylation. Phosphorylation is detected by immunofluorescence using anti-phospho-H1stone H2A.X antibody (Ser139) such as that provided by UpState Biotechnology.

Some macromolecules are not modified post-translationally, or, are modified constitutively—that is, their modifications do not change in response to external stimuli, environmental conditions, or other perturbants. By ‘respond’ we mean that a particular protein undergoes a change in modification status and/or subcellular distribution in response to a perturbation. Other post-translational modifications do respond and are induced by binding of an agonist, hormone or growth factor to a receptor which induces a signaling cascade or by a small molecule that activates an intracellular protein or enzyme. Other modifications can be inhibited, for example by binding of an antagonist or an antibody to a receptor thereby blocking a signaling cascade; by an siRNA, which silences a gene coding for a protein that is critical for a pathway; or by a drug that inhibits a particular protein within a pathway. These examples and the methods provided herein are meant to illustrate the breadth of the invention and are not limiting for the practice of the invention.

### EXAMPLE 1

In the first example, modification-state-specific antibodies were used to probe pathways within human cells. We created panels of quantitative, fluorescence assays for different states in live cells, where each state was a phosphoprotein, and tested the activities of known agents against the assay panels. We used fluorescence microscopy in combination with image analysis, such that the sub-cellular localization of each state could be assessed, enabling automated, “high-content” analyses. Specifically we assessed changes in the phosphorylation status of the pathway ‘sentinels’ by constructing high-content, immunofluorescence assays using phospho-specific antibodies targeted to the downstream proteins in the pathways of interest. Flow cytometry and fluorescence spectroscopy can also be used for this purpose, in cases where spatial resolution of the signal is not required. We demonstrate that the pattern of responses or “pharmacological profiles” detected by changes in intensity and/or compartment of the sentinel pair is related to the mechanism of action, specificity, and off-pathway effects of the drugs being tested; and that differences between drugs can readily be detected using this approach.

To demonstrate the general strategy and its application we studied multiple pathways that have been well-characterized in human cells. For the proof of principle we used three canonical signal transduction pathways: the cyclic AMP-dependent pathway; the ERK mitogen-activated protein kinase (MAPK) pathway; and the p38/MAPKAPK2 pathway. Each pathway has many other steps that have been documented in the biochemical literature; the diagram shows only a select few of the many proteins that participate in each pathway.

The beta-adrenergic receptor has been well characterized as a result of its pharmacological importance. This G-protein-coupled receptor (GPCR) is coupled to adenylyl cyclase via the small GTP-binding protein, Gs Binding of isoproterenol or other beta-adrenergic agonists to this receptor leads to activation of adenylate cyclase. When adenylyl cyclase is activated, it catalyses the conversion of ATP to cyclic AMP, which leads to an increase in intracellular levels of cyclic AMP. Cyclic AMP (cAMP) is a second messenger that activates the cyclic AMP-dependent protein kinase known as protein kinase A (PKA). Levels of cAMP are controlled through the regulation of the production of cAMP by adenylate cyclase, and the destruction of cAMP by phosphodiesterases. Adenylate cyclase can also be activated directly by agents such as forskolin, a diterpene that is widely used in studies aimed at dissecting intracellular signalling pathways. One of the best characterized substrates for PKA is the transcription factor CREB which is phosphorylated on serine133 (S133) in response to adrenergic agonists or other activators of PKA. Phosphorylation of CREB has been shown to increase its transcriptional activity for its target genes (Montminny et al).

ERK/MAPKs are key relay points in the transmission of growth factor-generated signals. This canonical growth factor receptor-stimulated pathway is initiated by a cell surface receptor, such as the epidermal growth factor (EGF) receptor tyrosine kinase. Activated EGF receptors bind to adaptor proteins and guanine nucleotide exchange factors, such as the protein SOS. SOS, in turn, activates small GTPases such as Ras, which then lead to phosphorylation and activation of a cascade of kinases including B-Raf and ERKs. By measuring the activity of a distal step in the pathway, such as phosphorylation of ERKs, the activity of upstream steps can be inferred. PD98059, a a relatively selective kinase inhibitor of the protein kinase known as MEK (MKK1/2), blocks events downstream of its target including the transcription factors ERK (shown in FIG. 4) and ELK. Given (a) a sufficiently specific anti-phospho-ERK antibody; (b) a cell type that is responsive to EGF; and (c) a sufficient quantity of PD98058; and (d) an immunofluorescence method that is capable of detecting phospho-ERK in intact cells, it should be possible to determine the effects of PD98059 on the amount and/or location of phospho-ERK in living cells.

The p38 serine/threonine protein kinase is the most well-characterized member of the MAP kinase family. It is activated in response to inflammatory cytokines, endotoxins, and osmotic stress. It shares about 50% homology with the ERKs. The upstream steps in activation of the cascade are not well defined. However, downstream activation of p38 occurs following its phosphorylation (at the TGY motif) by MKK3, a dual specificity kinase. Following its activation, p38 phosphorylates MAPKAPK2, which in turn phosphorylates and activates heat-shock proteins inclulding HSP27. SB203580 [4-(4-fluorophenyl)-2-(4-methylsulfinylphenyl)-5-(4-pyridyl)1H-imidazole] is a very specific inhibitor of p38 mitogen-activated protein kinase (MAPK) and is widely used as a tool to probe p38 MAPK function in vitro and in vivo.

We assessed the effects of the above-mentioned compounds on the three pathways and used the results to construct pharmacological profiles for the agents. Human cells (HEK293) were treated with drugs and the phosphorylation status of the three downstream proteins was assessed in the absence or presence of epidermal growth factor (EGF). Cells were then fixed and probed with antisera generated against the phosphorylated forms of CREB (S133), ERK1/2 (phospho T*EY*), or phospho Hsp27 (S78/S82). The ERK1/2 antibodies specifically recognize the MAPK/ERK1 and MAPK/ERK2 protein kinases only when they are phosphorylated on Threonine 202 and Tyrosine 204 in the activation loop. Phosphorylation of these amino acids has been shown to be necessary and sufficient for kinase activation, and therefore is a surrogate marker for activation of the kinases [Robbins et al.]. Changes in the level and sub-cellular localization of a phosphorylated protein following treatment with a drug would indicate a functional connection between the drug and the pathway of interest.

Details of the methods used are as follows. HEK293T cells were seeded in black-walled, poly-lysine coated 96-well plates (Greiner) at a density of 30,000/well. After 24 hours, cells in duplicate wells were treated with combinations of different drugs and stimulus as follows: (a) 20 micromolar PD98059, micromolar SB203580, or vehicle alone for 90 minutes; and (b) as for (a), but with 100 ng/ml hEGF added to the cells during the last 5 min of drug treatment. The drugs were purchased from Calbiochem and hEGF was from Roche. Four sets of cells treated as described were prepared. The cells were rinsed once with PBS and fixed with 4% formaldehyde for 10 min. The cells were subsequently permeabilized with 0.25% Triton X-100 in PBS and incubated with 3% BSA for 30 min to block non-specific antibody binding. Each of the 4 sets of identically treated cells were then incubated with rabbit antibodies against phosphorylated CREB (Ser133), Hsp27 (Ser82), or pERK (T202/Y204) (Cell Signaling Technology, Inc.). Control wells were incubated with bovine serum albumin (BSA) in PBS. The cells were rinsed with PBS and incubated with Alexa488 conjugated goat anti-rabbit secondary antibody (Molecular Probes). Cell nuclei were stained with Hoechst33342 (Molecular Probes).

Images were acquired using a Discovery-1 High Content Imaging System (Molecular Devices). Background fluorescence due to nonspecific binding by the secondary antibody was established with the use of cells that were incubated with BSA/PBS and without primary antibodies. Raw images in 16-bit grayscale TIFF format were analyzed using ImageJ API/library (http://rsb.info.nih.gov/ij/, NIH, MD). First, images from the fluorescence channels (Hoechst and Alexa 488) were normalized using the ImageJ built-in rolling-ball algorithm [S. R. Sternberg, Biomedical image processing. Computer, 16(1), January 1983]. Next a threshold was established to separate the foreground from background. An iterative algorithm based on Particle Analyzer from lmagej is applied to the thresholded Hoechst channel image (HI) to obtain the total cell count. The nuclear region of a cell (nuclear mask) is also derived from the thresholded HI. The positive particle mask is generated from the thresholded Alexa 488 image (YI). To calculate the global background (gBG), a histogram was obtained from the un-thresholded Alexa signaland the pixel intensity of the lowest intensity peak was identified as gBG. The Hoechst mask and Alexa mask are overlapped to define the correlated sub-regions of the cell. All means were corrected for the corresponding gBG. For each set of experiments (assay+drug treatment+treatment time), fluorescent particles from eight images were pooled. For each parameter, an outlier filter was applied to filter out those particles falling outside the range (mean±3SD) of the group. Finally the sample mean or control mean for each parameter was obtained from each filtered group. Results for drug treatments were normalized to the control for each experiment.

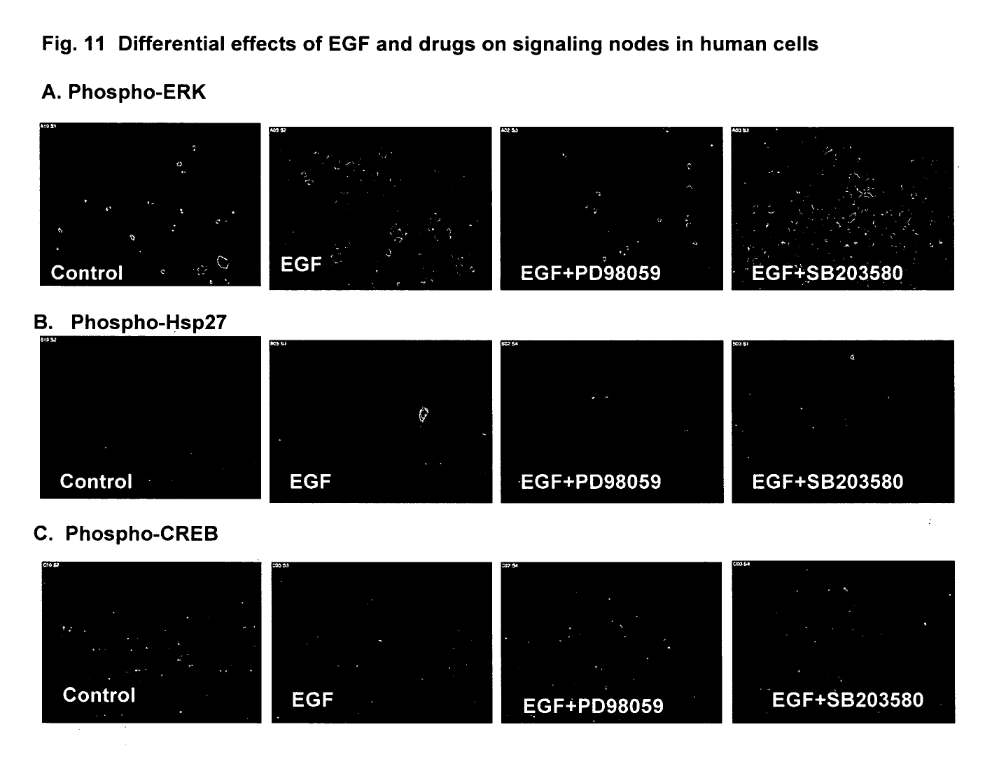

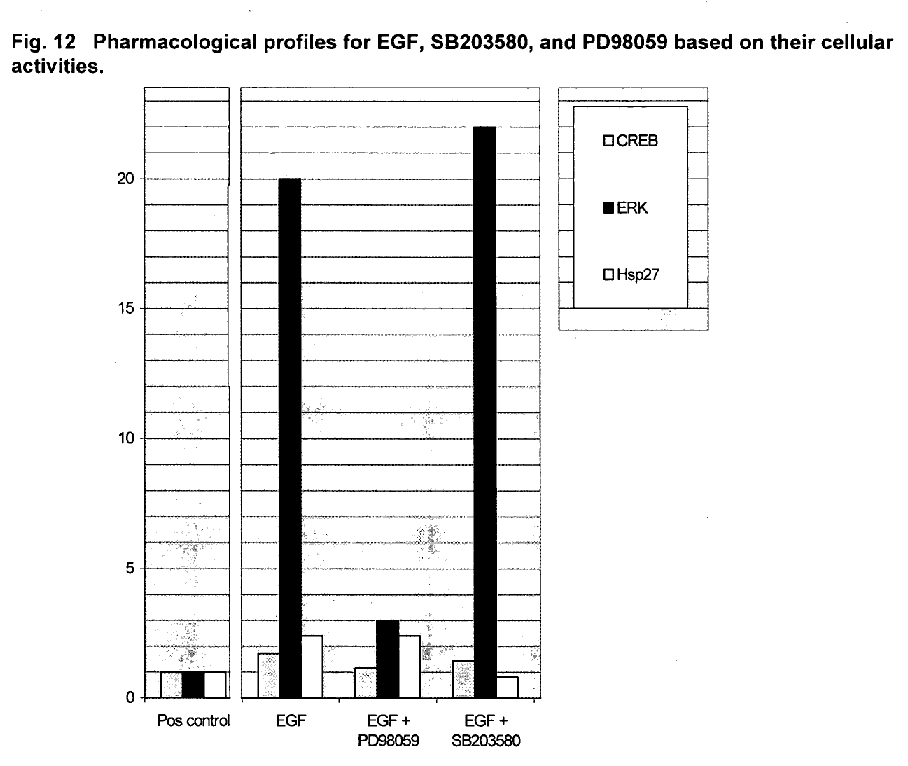

Results of the profiling are shown in FIGS. 11 and 12. The negative control wells (lower left) showed little or no signal with secondary antibody alone, demonstrating that the detection of phospho-CREB was accomplished with the phospho-specific antibody. In the presence of CREB phospho-specific antibody there was a clear fluorescence signal (control, upper left) that was localized predominantly at in a membrane/perinuclear pattern. EGF induced the formation of phospho-CREB, an effect that is consistent with cross-talk between the EGF-dependent and cyclic AMP-dependent pathways. The effect of EGF was reduced by PD98059, suggesting either that the PD compound has an off-pathway effect on the CREB pathway, or that the cross-talk between the EGF and CREB pathways occurs at a level below MEK (the target of PD98059). These results indicate that both direct and indirect effects of agonists and drugs on pathways can be assessed by assays of events downstream of the point of action of the agonist or drug, substantiating the premise that the connectivity of cellular networks can be exploited for use in identifying the spectrum of drug activities. The results also demonstrate the ability of the methodology to differentiate between agents that activate or inhibit pathways and those that have no effects on those pathways.

Differential activities of drugs on their expected targets/pathways were also observed. For example, EGF strongly stimulated the MAP kinase pathway, as expected, resulting in highly induced levels of ERKIMAP kinase phosphorylation (FIGS. 11-12). The compound PD98059, a known inhibitor of the kinase MEK, significantly blocked the phosphorylation of ERK in response to EGF, as expected. On the other hand, treatment of these cells with the p38-specific inhibitor SB203580 has no effect on EGF-stimulated ERK phosphorylation since SB203580 selectively acts on a pathway that is not connected to ERK. The results demonstrate the ability of the methodology to pinpoint on-pathway effects of drugs and to assess drug selectivity against pathways in human cells. This strategy also reveals cross-talk between pathways. EGF induced the p38 pathway as assessed by increased phospho-Hsp27 in EGF-treated cells and this response was blocked by the p38 inhibitor SB203580, demonstrating cross-talk between the EGF and p38 pathways at a level upstream of the site of action of SB203580. In contrast the MEK inhibitor PD98059 had no effect on EGF-induced Hsp27 phosphorylation, showing that PD98059 was selective for the MEK/ERK pathway.

The pharmacological profiles depicted in FIG. 12 demonstrate the similarities and differences between the agents. These pharmacological profiles can be used as fingerprints for drugs with certain mechanisms of action and selectivity. The fingerprints can be used to identify novel compounds with desired cellular effects and to eliminate compounds with undesired cellular effects. For example, using these methods, novel agents can be identified with cellular effects similar to EGF or to one of the kinase inhibitors. Establishing profiles for agents with known toxic or adverse effects will allow for attrition of novel compounds with similar (toxic or adverse) profiles.

### EXAMPLE 2

Here we demonstrate that both predicted and novel effects of known drugs and inhibitors can be deduced using the present invention (FIGS. 13-19). To represent a diversity of human cellular pathways, we created cell-based assays for 49 different states, where each state was a dynamic protein-protein complex representing one of the following processes: cell cycle control, DNA damage response, apoptosis, GPCR signalling, molecular chaperone interactions, cytoskeletal regulation, proteasomal degradation, mitogenesis, inflammation, and nuclear hormone receptor activation. The assays engineered for this study were protein-fragment complementation assays (PCAs) based on an intensely fluorescent mutant of YFP. We chose this reporter because the intense levels of autofluorescence allow the detection of complexes between full-length proteins expressed at low levels in human cells and the reconstituted YFP matures rapidly (9 minutes) allowing for detection of early effects on protein complexes. HEK293 cells were transiently co-transfected with a pair of PCA vectors, treated with vehicle or drug, and stimulated with agonists where indicated. The assays were categorized according to the sub-cellular localization of the fluorescent signal, for which we designed three different automated image analysis algorithms to measure changes in signal intensity across each sample population (8 images per sample; at least 1,600 cells per sample). Background-subtracted raw data were processed to determine drug-induced activity relative to pooled mean fluorescence of vehicle controls.

We first determined that the 49 assays responded appropriately to known pathway stimulators or inhibitors. The selected complexes are all documented to participate in their respective pathways, and are modulated by known transitions, including post-translational modifications, protein degradation or stabilization or protein translocation. Each drug caused unique patterns of activity, detected as increases or decreases in signal intensity or as a shift in the localization of the signal at a particular time of treatment compared to vehicle controls (FIG. 13). We detected unique temporal and spatial response patterns that were consistent with known mechanisms. For example, the beta-2 adrenergic receptor (beta2AR) is known to interact with beta-arrestin (beta-ARR2) following ligand-induced phosphorylation of the receptor by G protein-coupled receptor (GPCR) kinases (GRKs). We utilized the beta-Arr2:beta2AR PCA to evaluate the effects of drugs on GPCR signalling. The beta2AR agonist, isoproterenol (ISO), rapidly induced complex formation in a punctate cytoplasmic pattern, consistent with the binding of arrestin to the receptor and subsequent internalization via clathrin coated pits (FIG. 13). The structurally related beta-2AR agonists, salbutamol and clenbuterol, had similar effects. At later time points of drug treatments no further increases in the fluorescence signal were observed (FIG. 13B). This is consistent with the receptor down-regulation associated with prolonged exposure to agonists. In addition, the ISO-induced interaction of beta-Arr2:beta2AR, and the internalization of the receptor:arrestin complex was prevented by pretreatment with the inverse agonist, propranolol, as would be predicted (FIG. 13). An important feature of the approach is illustrated by these results for the beta-ARR2:beta-2AR assay, which was analyzed either in the absence or presence of isoproterenol. The example demonstrates that antagonists and inhibitors can be identified, by pretreating with the test or reference compound of interest and then stimulating a pathway with an agonist. Similarly, the first 5 assays in column 3 were stimulated with 500 nM camptothecin (CPT) for 960 minutes prior to the termination of the drug treatments. CPT induces DNA damage response pathways in human cells. We were able to detect changes in the distribution as well as intensity of protein complexes following treatment with drugs that target these pathways.

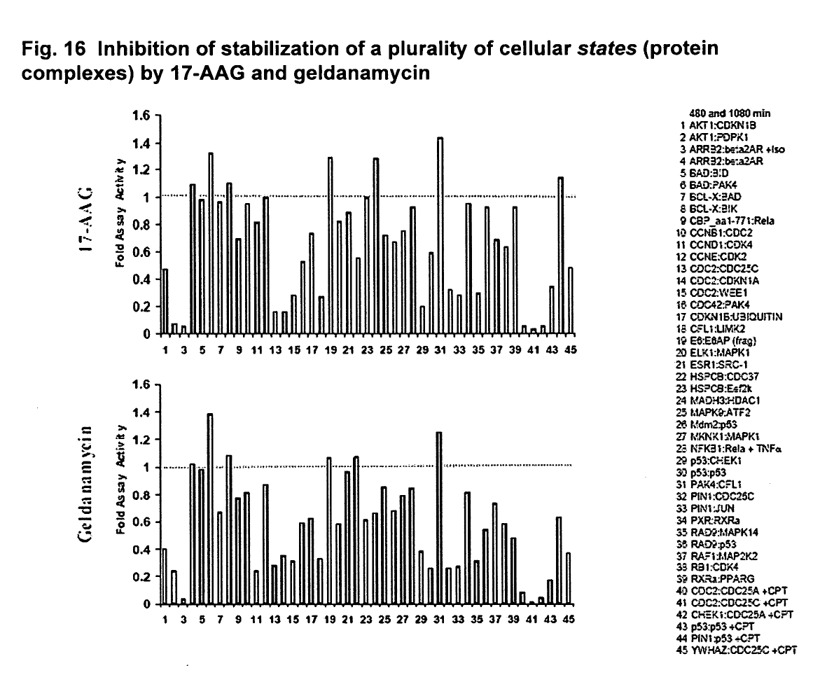

Specific states (protein complexes) reflect different functional aspects of pathway activity. Within a cell, the same state may have distinct dynamic roles in different cellular compartments. Consistent with this concept, we observed that assays reporting on cellular processes involving a common protein but localized in different cellular compartments had strikingly different responses to drugs. For example, we monitored drug activity on growth factor-mediated signal transduction pathways involving protein kinase B (Akt) by assessing the complex between Akt1 and phosphatidylinositol-3-dependent kinase (Pdk1). We also monitored complexes containing Akt1 and the cyclin-dependent kinase inhibitor, p27 (CDKN1B; kip1), which reports on cell cycle regulatory processes. In proliferating cells, phosphatidylinositol-3-kinase (PI3K) activation recruits Pdk and Akt kinases to the cell membrane. In vehicle treated cells, Akt1:Pdk1 complexes were predominantly localized at the cell membrane whereas Akt:p27 complexes were concentrated in the nucleus (FIG. 13). The distinct localizations of Akt:PDK1 and Akt:p27 illustrate that each assay reports on the biology of a specific complex rather than cellular pools of an individual protein; a feature crucial to detecting unique effects of drugs on different cellular transitions. In this example, the two distinct processes involving Akt were regulated differently. Wortmannin and LY294002, known inhibitors of PI3Ks, caused a rapid re-localization of Akt:Pdk1 from the membrane to the cytoplasm due to transportation of the complex, and a decrease in total Akt:Pdk1 complexes but induced nuclear Akt:p27 complexes. Conversely, Akt:p27 but not Akt:Pdk1 complexes were strongly inhibited by the kinase inhibitor BAY 11-7082, suggesting inhibition of enzyme activity by that compound. Finally, both Akt:Pdk1 and Akt:p27 complexes were negatively regulated 90 and 480 minutes after treatment with heat shock protein (HSP) inhibitors (Geldanamycin and 17-AAG) and by the non-specific kinase inhibitor indirubin-3′-monoxime (FIG. 13 and FIG. 16).

Additional examples, shown in FIG. 13, illustrate actions of drugs on diverse target classes and cellular processes. First, GTPase interactions with effector proteins are recognized as key molecular switches; a prototypical GTPase:kinase effector complex (HRas:Raf1) was inhibited by non-selective kinase inhibitors including BAY 11-7082. Second, LIM kinase 2 (Limk2) inactivates the actin depolymerizing factor cofilin, and thus represents kinase driven signalling processes controlling cytoskeletal morphology. These complexes were almost completely eliminated by the kinase inhibitor indirubin-3′-monoxime. Third, the cyclinD1:Cdk4 pair is an example of a cell cycle signalling node. Eight hours of treatment with the proteasome inhibitor, ALLN, resulted in accumulation of this complex in the nucleus, consistent with inhibition of the degradation of cyclin D1 by the 26S proteasome. Finally, we detected an increase in complexes of the ligand-activated transcription factors, RXR-alpha:PPAR-gamma following treatment with the known synthetic PPAR-gamma ligand, rosiglitazone (FIG. 13). In sum, drug effects on a wide range of cellular states and transitions could be detected with this approach. In addition, we observed distinct temporal patterns of compound activity, underscoring the importance of probing pathways at multiple early time points following drug addition (FIG. 13B).

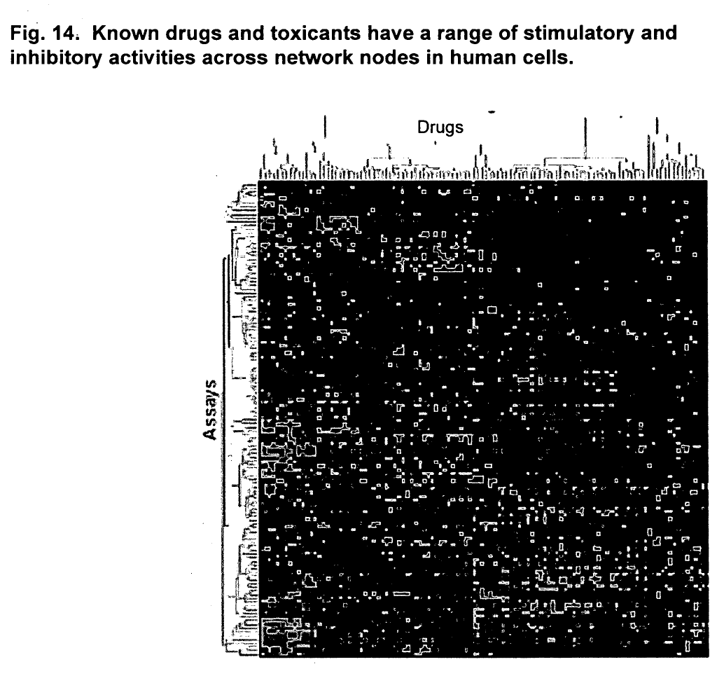

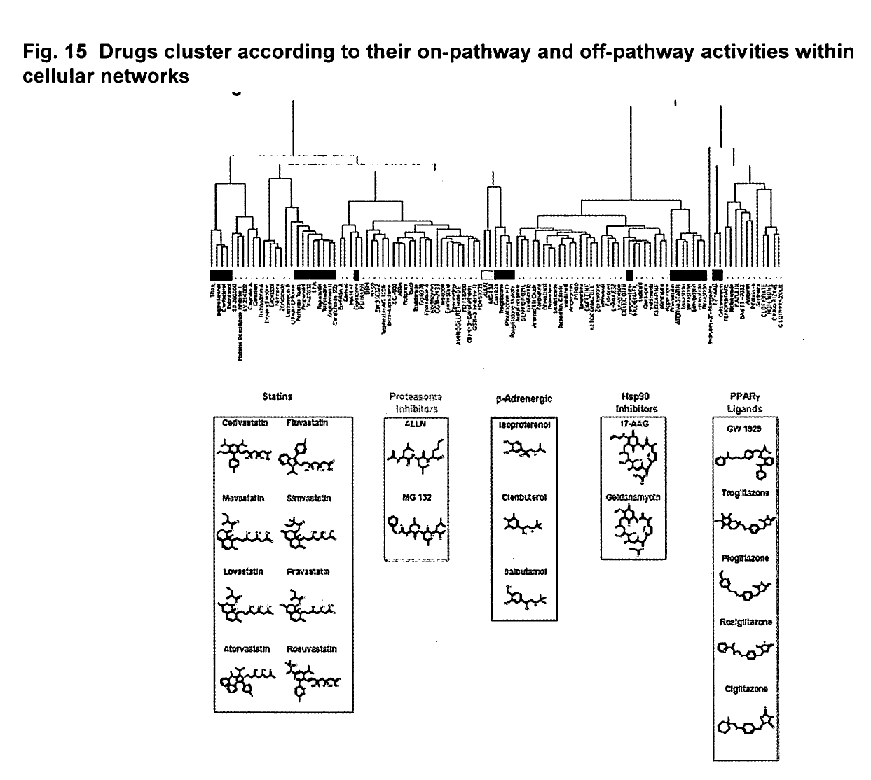

The examples presented above illustrate that effects of drugs on specific cellular processes were revealed by the spatial and temporal dynamics of protein complexes. In the expanded study, 107 drugs (Table 7) were tested against 49 assays (Table 6) that report on ten cellular processes targeting 6 therapeutic areas (cancer, inflammation, cardiovascular disease, diabetes, neurological disorders, infectious disease) or that function as general modulators of cellular mechanisms (e.g. protein transport). The drugs were chosen to represent the maximal diversity of mechanism and target, but were not intentionally chosen to act on the pathways being probed. Quantitative data for all drug/assay results are displayed in a color-coded matrix (FIG. 14). We calculated Euclidean distance metrics for each data point and applied hierarchical clustering algorithms to generate dendrograms; the matrix shown in FIG. 14 is clustered by drug (columns) and by assay and time point (rows). An expanded view of the drug cluster dendrogram is shown in FIG. 15.

Chemically related drugs were identified by their common assay activities. Remarkably, the drugs clustered primarily to known structure-target classes, in spite of the fact that the assays were not intentionally selected to report on pathways upon which the drugs act. In fact, many of the drugs are known to target proteins and pathway transitions that are not directly represented in the assay set. The results suggest that shared drug effects on cellular processes were revealed by the assessment of protein complex dynamics within the highly ramified cellular signalling networks. Several groups of structurally related drugs, with reportedly similar or identical cellular targets, generated functional clusters (FIG. 15). These included the proteasome inhibitors ALLN and MG-132, the HSP90 inhibitors geldanamycin and its semi-synthetic analog 17-(allylamino)-17-demethoxygeldanamycin (17-AAG), the beta-adrenergic GPCR agonists, isoproterenol, clenbuterol, and salbutamol, and the PPAR-gamma agonists, pioglitazone, rosiglitazone and troglitazone (FIG. 15).

Extensive similarities between the chemically related compounds geldanamycin and 17-AAG are evident in the matrix and the underlying data (FIGS. 15, 16). Some of the common activities represented assays involving HSP client proteins, such as those containing the Akt1 protein kinase (FIG. 13 and FIG. 16, bars #1&2), whereas other responses (e.g. modulation of DNA damage response assays and the beta-adrenergic GPCR assay) may reflect HSP90 activity on upstream components (FIG. 16).

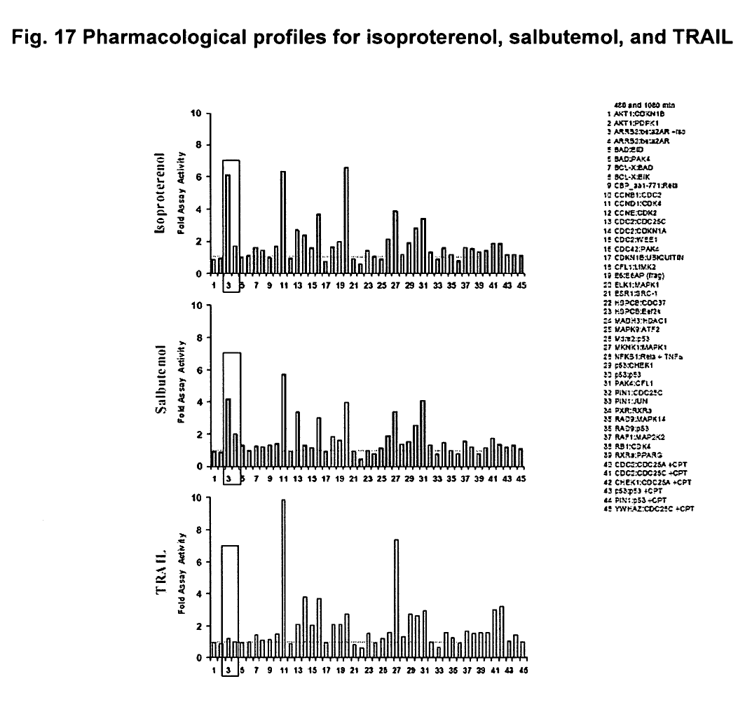

Chemically and functionally distinct drugs had similar assay activity. We also observed agents that have been thought to act on completely different biological processes, but which had similar activities in human cells. For example, the clustering of the beta-adrenergic receptQr agonists, isoproterenol, clenbuterol, and salbutamol, was predictable due to their stimulation of adrenergic receptor assays (FIGS. 15 and 17, bars #3 & 4); however, the protein cytokine TRAIL (a TNF-alpha-family ligand¹⁴) also clustered tightly with these agonists (FIGS. 15 and 17). Assay profiles representing TRAIL activity were strikingly similar to those for isoproterenol, clenbuterol, and salbutamol, with the notable exception that TRAIL had no activity on adrenergic GPCR assays (FIG. 4B, bar #s 3 & 4). Detailed examination of the biochemical activity of TRAIL and adrenergic agonists revealed surprising connections. Multiple assays involving mitogen-activated protein kinases (MAPKs) were strongly induced by adrenergic agonists and TRAIL, including ELK: MAPK1 (bar 20, FIG. 17), and MAPK1:MKNK1 (bar 27, FIG. 4B). These results are consistent with previous reports of adrenergic agonist- and TRAIL-induced MAPK activity. MAPK activity is associated with cyclin activation and these compounds also strongly induced cyclinD1:Cdk4 complexes following 8 hours of treatment (bar 11, FIG. 17).

Chemically distinct but functionally related drugs were identified by their common assay activity. For example, the PPAR-gamma agonists, pioglitazone, rosiglitazone and troglitazone clustered with the structurally distinct PPAR-gamma agonist, GW1929 (FIG. 15). Clustering of these compounds was driven in part by their similar activity on PPAR-gamma-related assays (PPAR-gamma:RXR-alpha and PPAR-gamma:SRC-1, FIG. 17 and not shown). However, these compounds also consistently and unexpectedly regulated other targets and pathways such as Rad9:MAPK14(p38) and cyclinD1:Cdk4 (data not shown).

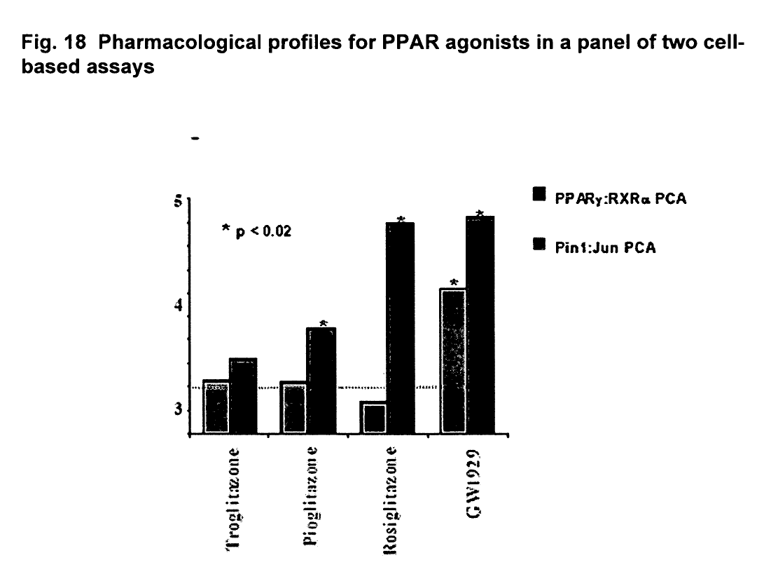

Off-pathway activity was detected among functionally related drugs. Similarities in activity between diverse compounds that share a common target are to be expected. However, we also observed differences between targeted molecules of similar potency, suggesting off-pathway activity. Such was the case for the PPAR□ agonist GW1929. We observed strong stimulation of the Pin1/Jun PCA with this compound, but not with the thiazolidinediones rosiglitazone, pioglitazone and troglitazone (FIG. 18). As all four compounds are potent PPAR-gamma agonists, the unique activity of GW1929 indicates that the observed off-target regulation of the Jun pathway was stimulated by some unique feature of the GW1929 chemical structure. These data generate test-able hypotheses of drug mechanisms that may be therapeutically important; for example, heightened activity of c-Jun activity indicates induction of pro-inflammatory pathways. It would be highly desirable to rapidly identify such potential off-pathway effects during the process of drug discovery. In addition, understanding the specific biochemical nature of the off-target activity would enable refinement of a chemical structure to address desirable or undesirable functional attributes of a molecule.

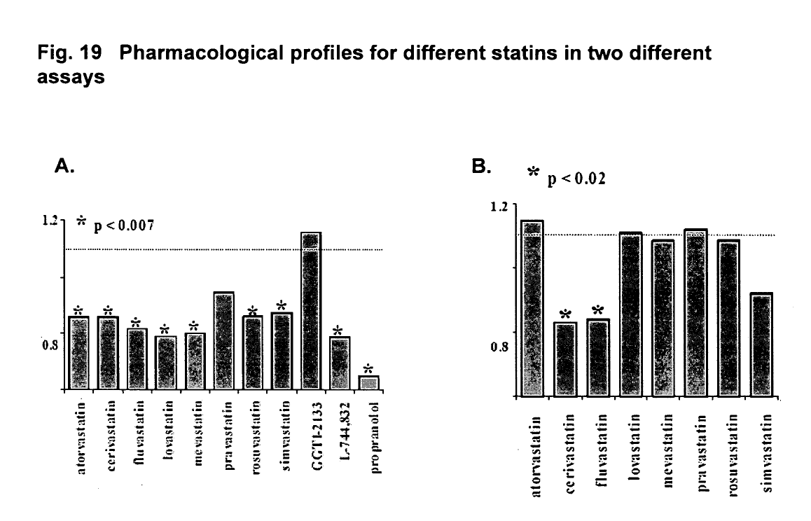

We identified secondary effects of statins that may be important for their therapeutic activity. The HMG Co-A reductase inhibitors (“statins”) are an important class of drugs that are widely employed for the reduction of serum cholesterol. HMG-Co-A reductase is the rate-limiting enzyme leading to cholesterol biosynthesis. We observed tight clustering of most statins despite the fact that no enzymes in the cholesterol biosynthetic pathway were directly represented in our assay panel. We did, however, observe assays in which all the statins that were tested had activity, such as the adrenergic receptor assay beta-Arr2:beta-2AR). This activity likely resulted from an effect downstream of statins, as inhibition of HMG-Co-A reductase also blocks the production of farnesyl pyrophosphate and geranyl-geranyl pyrophosphate, which are required for isoprenylation of specific proteins. In this case, the interaction of arrestins with GPCRs is induced by G-protein coupled receptor kinases (GRKs), some of which are directly regulated by isoprenylation. Interestingly, the beta-adrenergic receptor kinases (GRK2 and GRK3), which have been shown to recruit beta-arrestin to the receptor, were previously suggested to interact with geranyl-geranylated subunits. Our data showed a similar degree of inhibition with the farnesyl transferase inhibitor, L744832, but not the geranyl-geranyl transferase inhibitor, GGTI-2133, suggesting that farnesylation is responsible for beta-arrestin interactions with adrenergic receptors (FIG. 19A). The important role of these receptors in vascular biology suggests that the effects of statins in this assay may have relevance to their therapeutic activity in some settings. This example of pathway dissection demonstrates how assays define drug mechanisms, and how results with signature drugs (such as geranyl-geranyl or farnesyl transferase inhibitors) likewise provide a mechanistic framework for understanding assay activity.

We identified unique functional attributes of structurally related drugs. In addition to identifying similarities between compounds, we observed many examples of divergent activity of structurally related compounds on particular pathways. Among the statins, for example, rosuvastatin (marketed as Crestor™) was most distinct, demonstrating activity opposite from the other statins on cell cycle-related assays including CDKN1A(p21):Cdc2. Rosuvastatin also had little activity on the assay reporting on pro-inflammatory c-Jun (Pin1:Jun; FIG. 19B), whereas some statins did have profound effects on this assay. Cerivastatin (marketed as Baycol™) and fluvastatin (marketed as Lescol™) demonstrated pronounced inhibitory activity (FIG. 19B), consistent with previous observations of statin activity on the NF-kappa-B pathway, where cerivastatin was the most potent inhibitor. The effect of some, but not all statins on specific assays suggests off-pathway activity was responsible. Recent work suggests that pleiotropic effects of statins, including anti-inflammatory activity, are important contributors to their clinical activity. Rapid identification of biochemical differences in drugs of this class could expedite structure/function studies.

Taken together, the results presented here illustrate how the analysis of protein complexes, as reporters of individual cellular processes, reveal predicted as well as novel and potentially useful or dangerous drug effects. This approach does not always address the exact mechanism underlying the observed effects, but does generate testable hypotheses concerning potential on-pathway and off-pathway effects of drugs. The results have significance not only for understanding biology, but for understanding the complexity of drug activity in the context of living cells.

Some of the observed assay dynamics were responses to secondary or functional cellular activities, such as apoptosis or cell cycle progression, particularly at the longer (8-hour or 16-hour) time points. In this regard the approach is similar to other cell-based analyses, including gene expression analysis. However, a feature of the approach is the ability to capture both short-term and longer-term effects at multiple points within a pathway. In general, the shorter time points report on immediate effects of the compounds. In this study we recorded changes as early as 30 minutes following treatment; a time point likely to be meaningless for gene expression analysis.

The HEK 293 cell used in this study may not contain all the components of the biochemical pathways of interest, and may not be relevant to study drugs targeting a unique cellular process specific to a differentiated cellular lineage. However, the underlying assay strategy has been shown to be applicable to a broad range of mammalian as well as plant and bacterial cell lines. In addition, we have observed previously that unanticipated drug effects observed in a model cell line can predict interesting, pharmacologically and mechanistically important phenotypes in cells of different lineages, even if the functional phenotype is not observed in the model cell line used. These “hidden phenotypes” can point to unforeseen and potentially useful applications of drugs and to the formulation of novel hypotheses about drug actions.

The favored paradigm of modern drug discovery is the target-based, high-throughput screening (HTS) approach. The ability to discover large numbers of drug candidates from HTS has far out-stripped our ability to identify which compounds will be both efficacious and safe at later stages of the discovery process, or to predict other potential therapeutic applications. A rapid exclusion of compounds with potential deleterious effects (the so-called “fail-fast” strategy) is equally important. The approach we have described provides an efficient means to flag compounds for exclusion from further studies, or to redirect development to other therapeutic areas. Thus, we may both clarify our understanding of drug action in human cells and enhance the productivity of therapeutic discovery.

TABLE 6
List of 49 cell-based assays, their gene components and stimulation conditions

| | PCA Description | Stimulation | Genbank | Reporter 01 | Genbank | Reporter 02 |
|---|---|---|---|---|---|---|
| 1 | 14-3-3zeta:Cdc25C | 500 nM CPT; 16 hrs | BC003623 | C | NM_00179 | C |
| 2 | Akt1:p27 | | NM_00516 | N | NM_00406 | C |
| 3 | Akt1:Pdk1 | | NM_00516 | N | NM_00261 | C |
| 4 | Bad:Bid | | NM_00432 | N | NM_00119 | C |
| 5 | βArr2:β2AR | | NM_00431 | N | NM_00002 | C |
| 6 | Bad:Pak4 | | NM_00432 | C | NM_00588 | C |
| 7 | bArr2:b2AR + ISO | 2 μM ISO; 30 min | NM_00431 | N | NM_00002 | C |
| 8 | Bcl-xL:Bad | | NM_13857 | C | NM_00432 | N |
| 9 | Bcl-xL:Bik | | NM_13857 | N | NM_00111 | N |
| 10 | Cdc2:Cdc25A + CPT | 500 nM CPT; 16 hrs | NM_00178 | N | NM_00178 | C |
| 11 | Cdc2:Cdc25C | | NM_00178 | N | NM_00179 | C |
| 12 | Cdc2:Cdc25C + CPT | 500 nM CPT; 16 hrs | NM_00178 | N | NM_00179 | C |
| 13 | Cdc2:p21 | | NM_00178 | N | NM_00038 | N |
| 14 | Cdc2:Wee1 | | NM_00178 | N | NM_00951 | N |
| 15 | Cdc42:Pak4 | | NM_00179 | N | NM_00588 | C |
| 16 | Chk1:Cdc25A + CPT | 500 nM CPT; 16 hrs | NM_00127 | N | NM_00178 | C |
| 17 | Cofilin:Limk2 | | NM_00550 | C | NM_00556 | N |
| 18 | CyclinB:Cdc2 | | NM_03196 | N | NM_00178 | C |
| 19 | CyclinD:Cdk4 | | NM_05305 | N | NM_00179 | C |
| 20 | CyclinE:Cdk2 | | NM_00123 | N | NM_00171 | N |
| 21 | E6:E6AP | | AJ388069 | N | NM_13083 | C |
| 22 | Elk1:Mapk1 | | NM_00522 | C | NM_00274 | C |
| 23 | ESR1:SRC-1 | | NM_00012 | N | U40396 | N |
| 24 | H-Ras:Raf | | NM_00534 | N | NM_00288 | C |
| 25 | Hsp90:Cdc37 | | NM_00735 | C | NM_00706 | N |
| 26 | Hsp90:Eef2k | | NM_00735 | C | NM_00790 | N |
| 27 | IκB:p65 | 10 ng:ml TNF; 20 | NM_02052 | C | NM_00904 | N |
| 28 | MAPK9:ATF2 | | L31951 | N | NM_00188 | N |
| 29 | Mdm2:p53 | | BC0010793 | N | NM_00054 | C |
| 30 | Mknk1:Mapk1 | | NM_00368 | C | NM_00274 | C |
| 31 | ntCBP:p65 | | S66385 | N | NM_00904 | N |
| 32 | p27:UbiquitinC | | NM_00406 | N | NM_02100 | N |
| 33 | p50:p65 | 50 ng:ml TNF; 20 | NM_00391 | N | NM_00904 | N |
| 34 | p53:Chk1 | | NM_00054 | C | NM_00127 | N |
| 35 | p53:p53 | | NM_00054 | C | NM_00054 | C |
| 36 | p53:p53 + CPT | 500 nM CPT; 16 hrs | NM_00054 | C | NM_00054 | C |
| 37 | p70S6K:MAP3K8 | | NM_00316 | N | NM_00520 | N |
| 38 | PAK4:Cofilin | | NM_00588 | C | NM_00550 | C |
| 39 | Pin1:Cdc25C | | NM_00622 | C | NM_00179 | C |
| 40 | Pin1:Jun | | NM_00622 | C | NM_00222 | C |
| 41 | Pin1:p53 | | NM_00622 | C | NM_00054 | C |
| 42 | PXR:RXRa | | BC017304 | C | NM-002957 | C |
| 43 | Rad9:p38a | | NM_00458 | C | NM_00131 | N |
| 44 | Rad9:p53 | | NM_00458 | C | NM_00054 | N |
| 45 | Raf1:Map2k2 | | NM_00288 | C | D14592 | N |
| 46 | Rb1:Cdk4 | | BC040540 | C | NM_00179 | C |
| 47 | RXRa:PPARg | | NM-002957 | C | NM_13871 | C |
| 48 | Smad3:HDAC | | NM_00590 | N | NM_00496 | C |
| 49 | SRC-1:PPARγ | | U40396 | N | NM_13871 | C |

TABLE 6A
Additional cell-based assays engineered and optimized for
target and drug profiling and drug discovery

| | Assay_name |
|---|---|
| 50 | 4E-BP2/eIF4E |
| 51 | AKL3L/Guk1 |
| 52 | AKL3L/IPP2 |
| 53 | AMPK/HMGCR |
| 54 | annexin I/FPRL1 |
| 55 | ARF1/gamma-COP |
| 56 | ARNT/AhR |
| 57 | Asf1/Tlk1 |
| 58 | Axin/Cdk2 |
| 59 | β-catenin/LEF-1 |
| 60 | β-TrCP/β-catenin |
| 61 | CaM/Calcineurin A |
| 62 | CaM/DAPK2 |
| 63 | CaM/EGFR |
| 64 | CBP/β-catenin |
| 65 | CCR5/PKCα |
| 66 | Cdc37/Akt1 |
| 67 | Cdc6/Caspase 3 |
| 68 | Cdc6/Cdk2 |
| 69 | Cdk2/CyclinE |
| 70 | Chk1/Cdc25A |
| 71 | Chk1/Cdc25C |
| 72 | c-Jun/Fos |
| 73 | c-Jun/Jnk |
| 74 | c-Jun/CBP |
| 75 | c-Src/Grk2 |
| 76 | c-Src/PTPα |
| 77 | DGKα/PKCα |
| 78 | E2F1/DP-1 |
| 79 | E6/p53 |
| 80 | Elk1/Sumo-1 |
| 81 | ERα/SRC-1 |
| 82 | ETRA/βARR2 |
| 83 | G α (o)/Fz4 |
| 84 | Gβ4/Grk2 |
| 85 | GLUT4/Sumo-1 |
| 86 | Grk2/Gα(o) |
| 87 | Grk2/Gγ2 |
| 88 | GRK2/PKCα |
| 89 | GSK3β/PP2A |
| 90 | HDAC1/Smad3 |
| 91 | HDAC6/α-tubulin |
| 92 | Hsp90β/Akt1 |
| 93 | Hsp90β/Wee1 |
| 94 | ICAD/CAD |
| 95 | Stat5a/Stat5b |
| 96 | STK15/GUK1 |
| 97 | Syntaxin:Synaptobrevin2 |
| 98 | VPAC2/βARR2 |
| 99 | IPP2/RIPK2 |
| 100 | Ku70/Ku80 |
| 101 | LXRβ/RXRα |
| 102 | Mcm4/Cdk2 |
| 103 | Mdm2/NPM |
| 104 | Mdm2/p53 |
| 105 | Mdm2/PIAS1 |
| 106 | Mnk1/eIF4E |
| 107 | Mnk1/Erk2 |
| 108 | Mnk1/p38α |
| 109 | MST4/Caspase 3 |
| 110 | Myc/Max |
| 111 | Orc1/Orc2 |
| 112 | p21/CyclinB |
| 113 | p38α/Mkk3 |
| 114 | p50/p65 |
| 115 | p70S6K/Akt1 |
| 116 | p70S6K/Mkkk8 |
| 117 | p70S6K/RIPK2 |
| 118 | PAK6/Rac1 |
| 119 | Parvin-β/ILK |
| 120 | PCNAIPol δ (p50) |
| 121 | PCNA/Xrcc1 |
| 122 | PFKFB1/IPP2 |
| 123 | Pin1/β-catenin |
| 124 | Pin1/Cdc25C |
| 125 | Pin1/c-Jun |
| 126 | Pin1/p53 |
| 127 | PKCα/p47(phox) |
| 128 | PP1cβ/HDAC1 |
| 129 | PP1-r1a/Mkkk8 |
| 130 | PP1-r1a/Pdk1 |
| 131 | PTPα/Grb2 |
| 132 | PXR/RXRa |
| 133 | Rad17/Rad1 |
| 134 | Raf1/14-3-3ε |
| 135 | Raf1/Cdc37 |
| 136 | RalBP1/RalB |
| 137 | Rb1/Cyclophilin A |
| 138 | RGS2:Fz4 |
| 139 | RXRα/NURR1 |
| 140 | RXRα/THRα |
| 141 | Smad1/Smurf1 |
| 142 | SRC-1/FXR |
| 143 | SRC-1/TFCOUP2 |
| 144 | SRF/Elk1 |
| 145 | SSTR2/βARR2 |
| 146 | Vav/Cdc42 |
| 147 | Xrcc1/Ape1 |
| 148 | 14-3-3sigma/Bad |
| 149 | Akt1/GSK3β |
| 150 | AT1R/βARR2 |
| 151 | ATF1/Rps6ka4 |
| 152 | Bax/PML |
| 153 | β-catenin/GSK3β |
| 154 | β-Pix/PLC-γ |
| 155 | C5aR/βARR2 |
| 156 | CBP/CREB1 |
| 157 | CBP/Fos |
| 158 | CXCR3/βARR2 |
| 159 | DGK-α/c-Src |
| 160 | E2F1/Chk2 |
| 161 | EDG2/βARR2 |
| 162 | eIF4A/eIF4E |
| 163 | EP2/βARR2 |
| 164 | Fak1/SOCS-3 |
| 165 | Fz4/Grk2 |
| 166 | Gα15/Pthr1 |
| 167 | Gβ1/AT1R |
| 168 | GUK1/ACP1 |
| 169 | IKKβ/IKKγ |
| 170 | Insig-1/SCAP |
| 171 | ITGβ1/ITGα5 |
| 172 | LXA(4)R/βARR2 |
| 173 | Mdm2/PML |
| 174 | Mek1/Erk2 |
| 175 | Mevalonate Kinase/Mevalonate Kinase |
| 176 | NHE1/CHP |
| 177 | NLK/c-Myb |
| 178 | NOSTRIN/eNos |
| 179 | p53/Chk2 |
| 180 | PAR-1/βARR2 |
| 181 | PCNA/Ape1 |
| 182 | Phkg1/CaM |
| 183 | PKCα/ACE |
| 184 | PKCα/SphK1 |
| 185 | PPARα/RXRα |
| 186 | PPARδ/RXRα |
| 187 | Pthr/βARR2 |
| 188 | Raf1/Mek1 |
| 189 | Raf-1/PP2A |
| 190 | RARβ/RXRα |
| 191 | Rb/Cdk4 |
| 192 | RGS2/G-α-i2 |
| 193 | Smad3/Akt1 |
| 194 | Smad3/Cdk2 |
| 195 | SRF/Sumo-1 |
| 196 | Stat1/Stat1 |

TABLE 7
List of 107 drugs and their screening doses for Example 2 (FIGS. 13-19)

| DRUG | Concentration | |
|---|---|---|
| (S)-(+)-Camptothecin | 500 | nM |
| 17-AAG | 5 | μM |
| Acetyl ceramide | 10 | μM |
| ALLN | 25 | μM |
| Aminoglutethimide | 30 | μM |
| Angiogenin | 100 | ng:ml |
| Angiotensin II | 300 | nM |
| Apigenin | 50 | μM |
| Arsenic(III) Oxide | 5 | μM |
| Atorvastatin | 30 | μM |
| ATRA | 5 | μM |
| BAY 11-7082 | 10 | μM |
| β-Lapachone | 2 | μM |
| Bicalutamide | 500 | nM |
| Brefeldin A | 50 | mg:ml |
| Caffeine | 50 | μM |
| Calyculin A | 2 | nM |
| Celecoxib | 10 | μM |
| Cerivistatin | 30 | μM |
| Ciglitazone | 15 | μM |
| Cilostazol | 2 | μM |
| Cinnarizine | 15 | μM |
| Ciprofibrate | 30 | μM |
| Clenbuterol | 2 | μM |
| Clofibrate | 30 | μM |
| Clotrimazole | 10 | μM |
| Clozapine | 2 | μM |
| DBH | 5 | μM |
| Dexamethasone | 500 | nM |
| Epothilone A | 100 | nM |
| Estrogen | 500 | nM |
| Exemestane | 1.50 | μM |
| Fenofibrate | 30 | μM |
| Fluvastatin | 30 | μM |
| Fulvestrant | 500 | nM |
| Geldanamycin | 30 | μM |
| Genistein | 12.5 | μM |
| Gemfibrozil | 12.5 | μM |
| GGTI-2133 | 5 | μM |
| Gleevec | 10 | μM |
| Gö 61076 | 100 | nM |
| GSK-3 Inh. II | 1 | μM |
| GW1929 | 3 | μM |
| H-89 | 2 | μM |
| HA14-1 | 2 | μM |
| Indirubin-3′- | 10 | μM |
| Isoproterenol | 2 | μM |
| Ketoconazole | 30 | μM |
| L-744,832 | 10 | μM |
| Leptomycin B | 10 | ng:ml |
| Letrozole | 1.50 | μM |
| Levamlsole | 10 | μM |
| Lithium Chloride | 1000 | μM |
| Lovastatin | 30 | μM |
| LPA | 5 | μM |
| LY 294002 | 25 | μM |
| Mevastatin | 30 | μM |
| MG 132 | 1 | μM |
| Milrinone | 200 | nM |
| MS-275 | 10 | μM |
| Niclosamide | 1 | μM |
| Olanzapine | 2 | μM |
| Paroxetine | 10 | μM |
| Patulin | 10 | μM |
| PD 158780 | 1 | μM |
| PD 107059 | 20 | μM |
| PD153035 | 200 | nM |
| Pertussis Toxin | 100 | ng:ml |
| Pifithrin-a | 50 | μM |
| Pioglitazone | 15 | μM |
| Pravastatin | 30 | microM |
| Propanolol | 2 | μM |
| PTPBS | 500 | nM |
| Quetiapine | 2 | μM |
| Raloxifene | 500 | nM |
| Rapamycin | 250 | nM |
| Risperidone | 2 | μM |
| Rofecoxib | 10 | μM |
| Rolipram | 25 | μM |
| Roscovitine | 5 | μM |
| Rosiglitazone | 15 | μM |
| Rosuvastatin | 30 | μM |
| Rotenone | 300 | nM |
| Salbutamol | 2 | μM |
| Sarafotoxin S6b | 100 | nM |
| SB 203580 | 25 | μM |
| SC-560 | 250 | nM |
| Sertraline | 10 | μM |
| Sildenafil | 1 | μM |
| Simvastatin | 30 | μM |
| Tadalafil | 1 | μM |
| Tamoxifen | 500 | nM |
| Taxol | 2.5 | μM |
| Terfenadine | 10 | μM |
| Thalidomide | 250 | μM |
| Toremifene | 500 | nM |
| TRAIL | 50 | ng:ml |
| Trichostatin A | 5 | μM |
| Troglitazone | 15 | μM |
| Tyrphostin AG | 5 | μM |
| Valdecoxib | 10 | μM |
| Vardenafil | 1 | μM |
| Wortmannin | 500 | nM |
| Y-27632 | 25 | μM |
| Ziprasidone | 2 | μM |
| ZM 336372 | 5 | μM |
| Zonisamide | 5 | μM |

Detailed Methods for Example 2

Reporter fragments for PCA: were generated by oligonucleotide synthesis (Blue Heron Biotechnology, Bothell, Wash.). Synthetic fragments coding for polypeptide fragments YFP[1]and YFP[2] (corresponding to amino acids 1-158 and 159-239 of YFP) were generated. PCR mutagenesis then was used to generate the mutant fragments IFP[1] and IFP[2]. The IFP[1] fragment corresponds to YFP[1]-(F46L, F64L, M153T) and the IFP[2] fragment corresponds to YFP[2]-(V163A, S175G). These mutations have been shown to increase the fluorescence intensity of the intact YFP protein. YFP and IFO fusion constructs were generated as previously described. Transient transfections: HEK293 cells were maintained in MEM alpha medium (Invitrogen) supplemented with 10% FBS (Gemini Bio-Products), 1% penicillin, and 1% streptomycin, and grown in a 37° C. humidified incubator equilibrated to 5% CO2. Twenty-four hours prior to transfections cells were seeded into 96 well ploy-D-Lysine coated plates (Greiner) using a Multidrop 384 peristaltic pump system (Thermo Electron Corp., Waltham, Mass.) at a density of 7,500 cells per well. Up to 10 ng of the complementary YFP or IFP-fragment fusion vectors were co-transfected using Fugene 6 (Roche) according to the manufacturer's protocol. The PCA pairs screened in this study, and corresponding gene and reporter fragment information, are listed in Table 1. Twenty-four or 48 hours after transfection, cells were screened against a panel of drugs as described below. For p50:p65, beta-Arr2:beta2AR and Akt1:pDK1, stable cell lines were generated as described in References. Drug study: The 49 PCAs (gene names, GenBank references, and stimulation conditions are listed in Table 6) were screened in duplicate against a panel of 107 drugs (names and doses are listed in Table 7). Drug concentrations were chosen based on published cellular IC50's and were further refined to ensure lack of toxicity in HEK293 cells, based on lactate dehydrogenase (LDH) toxicity analyses. All liquid handling steps were performed using the Biomek FX (Beckman Instruments, Fullerton, Calif.). Cells expressing the PCA pairs were incubated in cell culture medium containing drugs for 30 min., 90 min., and 8 hours, or in some cases for 18 hours. For some assays cells were treated with agonists immediately prior to the termination of the assay (refer to Table 1 for stimulants). Following drug treatments cells were simultaneously stained with 33 micrograms/ml Hoechst 33342 (Molecular Probes) and 15 micrograms/ml TexasRed-conjugated Wheat Germ Agglutinin (WGA; Molecular Probes), and fixed with 2% formaldehyde (Ted Pella) for 10 minutes. Cells were subsequently rinsed with HBSS (Invitrogen) and maintained in the same buffer during image acquisition. YFP, Hoechst, and Texas Red fluorescence signals were acquired using the Discovery-1 automated fluorescence imager (Molecular Devices, Inc.) equipped with a robotic arm (CRS Catalyst Express; Thermo Electron Corp., Waltham, Mass.). The following filter sets were used to obtain images of 4 non-overlapping populations of cells per well: excitation filter 480+/−40 nm, emission filter 535+/−50 nm (YFP); excitation filter 360+/−40 nm, emission filter 465+/−30 nm (Hoechst); excitation filter 560+/−50 nm, emission filter 650+/−40 nm (Texas Red). All treatment conditions were run in duplicate yielding a total of 8 images for each wavelength and treatment condition. Fluorescence image analysis: Raw images in 16-bit grayscale TIFF format were analyzed using ImageJ API/library (http://rsb.info.nih.gov/ij, NIH, MD). First, images from all 3 fluorescence channels (Hoechst, YFP, and Texas Red) were normalized using the ImageJ built-in rolling-ball algorithm.²⁹ Next a threshold was established to separate the foreground from background. An iterative algorithm based on Particle Analyzer from ImageJ was applied to the thresholded Hoechst channel image (HI) to obtain the total nuclear area. The nuclear region of a cell (nuclear mask) was also derived from the thresholded HI. A WGA mask was generated similarly from the thresholded Texas Red image (TRI), and a positive particle mask was generated based on the thresholded YFP images (YI). Images were analyzed by 1 of 3 algorithms depending on the nature of the fluorescent signal: 1) The positive particle mask was used to sample positive YFP pixels, and mean fluorescence intensities were calculated; 2) The nuclear region mask was used to sample pixels within the nuclear region. Nuclear pixels above a manually established gating value were sampled to determine mean nuclear fluorescence and total nuclear fluorescence (standardized to Hoechst area); 3) All pixels above the above a manually established gating value were sampled to determine mean total fluorescence and total fluorescence standardized to Hoechst area. All values were corrected for background and An outlier filter was applied to filter out scan data that fell outside the range (mean±3SD) of the group. A mean was obtained from each filtered group. Data Analysis and Clustering: In the clustering matrix (FIG. 2) each row represents a combination of a unique assay, a drug treatment time and time point. Each column represents a unique drug. Each data point was formed by taking the log of the ratio of the sample to the control. Every row and column carries equal weight. The Ward hierarchical clustering algorithm¹³ and Euclidean distance metrics were used for clustering the drugs over the matrix. The hierarchical clustering was performed using the open-source statistics software package R (http://www.r-project.org/). For display purposes the data were colour coded to illustrate relative differences within an assay. For each row, the dynamic range of the values (reported as log Ratio sample/control) was separated into 9 levels. An increase relative to the control value was displayed as green and a decrease was displayed as red. Each colour was further divided into four levels: level 1 (>75%), level 2 (>=50% and <75%), level 3 (>=25% and <50%), level 4 (>0 and <25%). Level one was displayed as the brightest hue and level 2 as the darker. Level 3 and 4 were shaded in black.

### EXAMPLE 3

Small interfering RNA (siRNA) represents an exciting new chemical class of compounds for human therapeutics. The first human clinical trials of an siRNA compound are now in progress. The technology of RNA interference (RNAi) also represents a breakthrough in efforts to identify, validate and link genes to specific cellular processes and to identify optimal targets for the development of human therapeutics. In addition to studying the functional consequences of gene targeting in living cells, the ultimate goal of such studies is to understand the biochemical connection between the target that is silenced and the effect that is observed. RNAi strategies rely on the property of double-stranded RNA (dsRNA) to activate the endogenous cellular process of highly specific RNA degradation, and are generally employed to link specific genes to their functional roles within the cellular signaling network and to identify proteins of potential therapeutic or diagnostic relevance. However, utilization of phenotypic or gene expression analyses in concert with RNAi only allows inferences regarding the connections between targeted genes and their biochemical roles. A direct and systematic analysis of the effects of gene silencing on signaling pathways has yet to be described.

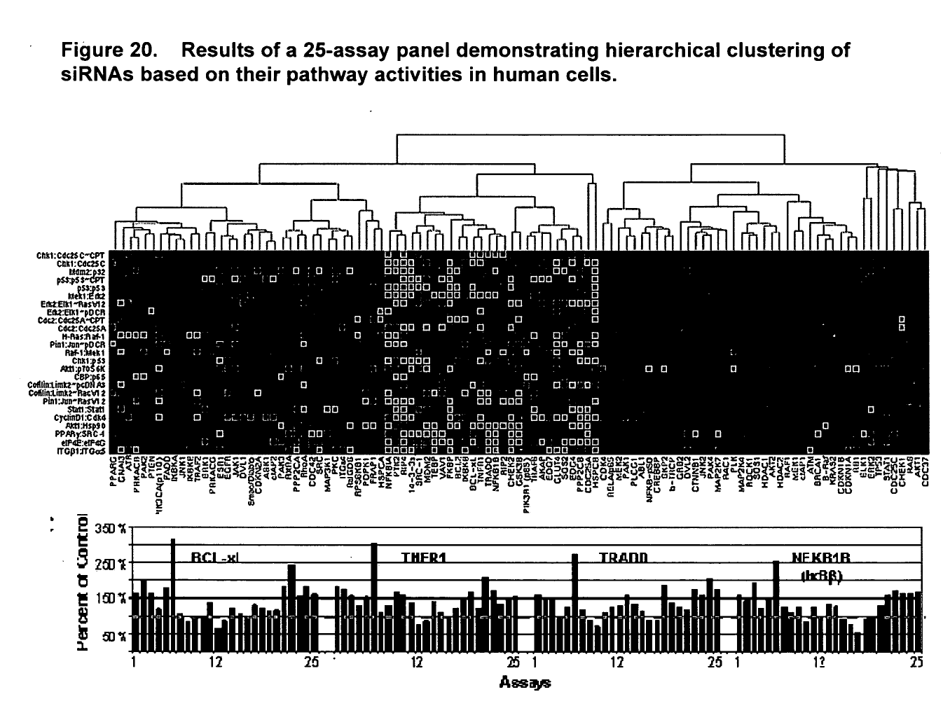

We applied network biology to monitor siRNA-induced transitions which report on information flow through signal transduction pathways. The effects of silencing 107 different targets in key signaling pathways and processes in living human cells were assessed (FIGS. 20-24). A diverse set of fluorescence-based protein-fragment complementation assays (PCAs) was used to report on the activity of the PI3K/Akt-, RAS/MAPK- and NF-kappa-B-mediated pathways; and pathways underlying DNA damage response, cell cycle, apoptotic regulators and nuclear hormone receptor signaling. The results support our network biology strategy for identifying drug mechanisms of action.

TABLE 8
Panel of 107 siRNAs used for pharmacological profiling

| siRNA | | | | Gene | Dharmacon |
|---|---|---|---|---|---|
| No. | siRNA Name | Protein Target | Pathway/Classification | Accession | Product Number |
| 1 | PTEN | PTEN | PI3K/AKT | NM_000314 | M-003023-00-05 |
| 2 | PIK3CA | p110a PI3K | PI3K/AKT | NM_006218 | M-003018-00-05 |
| 3 | PIK3R1 | p85a PI3K | PI3K/AKT | NM_181523 | M-003020-00-05 |
| 4 | PDPK1 | Pdk1 | PI3K/AKT | NM_002613 | M-003558-00-05 |
| 5 | AKT1 | Akt1 | PI3K/AKT | NM_005163 | M-003000-00-05 |
| 6 | AKT2 | Akt2 | PI3K/AKT | NM_001626 | M-003001-00-05 |
| 7 | GSK3B | Gsk3b | PI3K/AKT | NM_002093 | M-003010-00-05 |
| 8 | RPS6KB1 | p70S6K | PI3K/AKT | NM_003161 | M-003616-00-05 |
| 9 | FRAP1 | FRAP/TOR | PI3K/AKT | NM_004958 | M-003008-01-05 |
| 10 | FKBP | FK506-BP (12 kD) | PI3K/AKT | NM_054014 | M-005183-00-05 |
| 11 | HSPCA | Hsp90a | Hsp90/co-chaperones | NM_005348 | M-005186-00-05 |
| 12 | HSPCB | Hsp90b | Hsp90/co-chaperones | NM_007355 | M-005187-00-05 |
| 13 | CDC37 | Cdc37 | Hsp90/co-chaperones | NM_007065 | M-003231-00-05 |
| 14 | TEBP | p23 | Hsp90/co-chaperones | NM_006601 | M-005192-00-05 |
| 15 | cIAP1 | cIAP1 | Apoptosis | NM_001166 | M-004390-00-05 |
| 16 | cIAP2 | cIAP2 | Apoptosis | NM_001165 | M-004099-00-05 |
| 17 | Smac/Diablo | Smac/Diablo | Apoptosis | NM_019887 | M-004447-00-05 |
| 18 | BCL2 | BCL2 | Apoptosis | NM_000633 | M-003307-00-05 |
| 19 | BCL-xL | BCL-xL | Apoptosis | NM_138578 | M-003458-00-05 |
| 20 | TNFR1 | TNF-R | NFkB signaling | NM_001065 | M-005197-00-05 |
| 21 | RIP2 | RIP2 | NFkB signaling | NM_003821 | M-005370-00-05 |
| 22 | RIP4 | RIP4 | NFkB signaling | NM_020639 | M-005308-00-05 |
| 23 | TRADD | TRADD | NFkB signaling | NM_003789 | M-004452-00-05 |
| 24 | FADD | FADD | NFkB signaling | NM_003824 | M-003800-00-05 |
| 25 | TRAF2 | TRAF2 | NFkB signaling | NM_021138 | M-005198-00-05 |
| 26 | TRAF6 | TRAF6 | NFkB signaling | NM_004620 | M-004712-00-05 |
| 27 | IKBKA | IKKa | NFkB signaling | NM_001278 | M-003473-00-05 |
| 28 | IKBKB | IKKb | NFkB signaling | XM_032491 | M-004120-00-05 |
| 29 | IKBKE | IKKe | NFkB signaling | NM_014002 | M-003723-00-05 |
| 30 | NFKBIA | IkBa | NFkB signaling | NM_020529 | M-004765-00-05 |
| 31 | NFKB1B | IkBb | NFkB signaling | NM_002503 | M-004764-00-05 |
| 32 | RELA/p65 | NFkB-p65 | NFkB signaling | NM_021975 | M-003533-00-05 |
| 33 | NFKB-p50 | NFkB-p50 | NFkB signaling | NM_003998 | M-003520-00-05 |
| 34 | CREBBP | CBP | NFkB signaling | NM_004380 | M-003477-00-05 |
| 35 | HDAC1 | HDAC1 | Nuclear Hormone Receptor | NM_004964 | M-003494-00-05 |
| 36 | HDAC2 | HDAC2 | Nuclear Hormone Receptor | NM_001527 | M-003495-00-05 |
| 37 | SRC-1 | SRC-1 | Nuclear Hormone Receptor | U90661.1 | M-005196-00-05 |
| 38 | ESR1 | ERa | Nuclear Hormone Receptor | NM_000125 | M-003489-00-05 |
| 39 | PPARG | PPARg | Nuclear Hormone Receptor | NM_138712 | M-003436-00-05 |
| 40 | RXRA | RXRa | Nuclear Hormone Receptor | NM_002957 | M-003443-00-05 |
| 41 | SKP2 | Skp2 | Cell cycle/damage response | NM_005983 | M-003541-00-05 |
| 42 | b-TRCP | β-TRCP | Cell cycle/damage response | NM_033637 | M-003463-00-05 |
| 43 | MDM2 | Hdm2 | Cell cycle/damage response | NM_002392 | M-003279-00-05 |
| 44 | TP53 | p53 | Cell cycle/damage response | NM_000546; | M-003329-00-05 |
| | | | | M14695 | |
| 45 | ATM | ATM | Cell cycle/damage response | NM_000051 | M-003201-00-05 |
| 46 | ATR | ATR | Cell cycle/damage response | NM_001184 | M-003202-01-05 |
| 47 | ABL1 | c-ABL | Cell cycle/damage response | NM_007313 | M-003100-01-05 |
| 48 | BRCA1 | Brca1 | Cell cycle/damage response | NM_007295 | M-003461-00-05 |
| 49 | CHEK1 | Chk1 | Cell cycle/damage response | NM_001274 | M-003255-01-05 |
| 50 | CHEK2 | Chk2 | Cell cycle/damage response | NM_007194 | M-003256-00-05 |
| 51 | CDC25A | Cdc25A | Cell cycle/damage response | NM_001789 | M-003226-00-05 |
| 52 | CDC25C | Cdc25C | Cell cycle/damage response | NM_001790 | M-003228-00-05 |
| 53 | PLK | Plk | Cell cycle/damage response | NM_005030 | M-003290-00-05 |
| 54 | CDK4 | Cdk4 | Cell cycle/damage response | NM_000075 | M-003238-00-05 |
| 55 | RB1 | Rb | Cell cycle/damage response | NM_000321 | M-003296-00-05 |
| 56 | CDKN1A | Cip/p21 | Cell cycle/damage response | NM_078467; | M-003471-00-05 |
| | | | | NM_000389 | |
| 57 | CDKN1B | Kip/p27 | Cell cycle/damage response | NM_004064 | M-003472-00-05 |
| 58 | CDKN2A | INK4/p16 | Cell cycle/damage response | NM_000077 | M-005191-00-05 |
| 59 | 14-3-3s | 14-3-3s | Cell cycle/damage response | NM_006142 | M-005180-00-05 |
| 60 | STAT1 | Stat1 | Ras/MAPK | NM_007315 | M-003543-00-05 |
| 61 | JAK1 | Jak1 | Ras/MAPK | NM_002227 | M-003145-01-05 |
| 62 | EGFR | EGFR | Ras/MAPK | NM_005228 | M-003114-01-05 |
| 63 | SRC | c-Src | Ras/MAPK | NM_005417 | M-003175-01-05 |
| 64 | GRB2 | Grb2 | Ras/MAPK | NM_002086 | M-004112-00-05 |
| 65 | SOS1 | Sos1 | Ras/MAPK | NM_005633 | M-005194-00-05 |
| 66 | SOS2 | Sos2 | Ras/MAPK | XM_043720 | M-005195-00-05 |
| 67 | PLCG1 | PLC-g | Ras/MAPK | NM_002660 | M-003559-00-05 |
| 68 | RaIGDS | RaIGDS | Ras/MAPK | NM_006266 | M-005193-00-05 |
| 69 | RAS | H-Ras | Ras/MAPK | NM_005343 | M-004142-00-05 |
| 70 | KRAS2 | K-Ras | Ras/MAPK | NM_004985 | M-005069-00-05 |
| 71 | RAF1 | c-Raf | Ras/MAPK | NM_002880 | M-003601-00-05 |
| 72 | B-Raf | B-Raf | Ras/MAPK | NM_004333 | M-003460-00-05 |
| 73 | MEK1 | Mek1 | Ras/MAPK | NM_002755 | M-003571-00-05 |
| 74 | MEK2 | Mek2 | Ras/MAPK | NM_030662 | M-003573-00-05 |
| 75 | ERK2 | Erk2 | Ras/MAPK | M84489 | M-003555-02-05 |
| 76 | ERK1 | Erk1 | Ras/MAPK | AK091009 | M-003592-00-05 |
| 77 | ELK1 | Elk1 | Ras/MAPK | NM_005229 | M-003885-00-05 |
| 78 | VAV1 | Vav1 | Rho family | NM_005428 | M-003935-00-05 |
| 79 | CDC42 | Cdc42 | Rho family | NM_001791 | M-005057-00-05 |
| 80 | RAC1 | Rac1 | Rho family | NM_018890 | M-003560-00-05 |
| 81 | PAK1 | Pak1 | Rho family | NM_002576 | M-003521-00-05 |
| 82 | PAK2 | Pak2 | Rho family | NM_002577 | M-003597-00-05 |
| 83 | PAK3 | Pak3 | Rho family | AF068864 | M-003614-00-05 |
| 84 | PAK4 | Pak4 | Rho family | NM_005884 | M-003615-00-05 |
| 85 | RhoA | RhoA | Rho family | NM_001664 | M-004549-00-05 |
| 86 | ROCK1 | p160-ROCK | Rho family | NM_005406 | M-003536-00-05 |
| 87 | MAP3K1 | MEKK1 | JNK/SAPK signaling | XM_042066 | M-003575-00-05 |
| 88 | MAP2K7 | MKK7/JNKK2 | JNK/SAPK signaling | NM_005043 | M-004016-00-05 |
| 89 | ASK1 | MEKK5 | JNK/SAPK signaling | E14699 | M-004539-00-05 |
| 90 | MAP2K4 | MKK4/JNKK1 | JNK/SAPK signaling | NM_003010 | M-003574-00-05 |
| 91 | JNK2 | JNK2 | JNK/SAPK signaling | L31951 | M-003766-00-05 |
| 92 | JNK1 | JNK1 | JNK/SAPK signaling | L26318 | M-003765-00-05 |
| 93 | ITGa4 | ITGa4 | Ras/MAPK | L12002 | M-005189-00-05 |
| 94 | PTK2 | FAK | Ras/MAPK | NM_005607 | M-003164-01-05 |
| 95 | CTNNB1 | β-catenin | Wnt pathway | NM_001904 | M-003482-00-05 |
| 96 | DVL1 | Dsh1 | Wnt pathway | U46461 | M-004068-00-05 |
| 97 | DVL2 | Dsh2 | Wnt pathway | NM_004422 | M-004069-00-05 |
| 98 | EDG4 | Edg-4/LPA2 | GPCR/G-protein | AF233092 | M-004602-00-05 |
| 99 | EDG7 | Edg-7/LP-A3 | GPCR/G-protein | NM_012152 | M-004895-00-05 |
| 100 | GNAI3 | Gai-3 | GPCR/G-protein | NM_006496 | M-005184-00-05 |
| 101 | GLUT4 | GLUT4 | PKA/PKC signaling | NM_001042 | M-005185-00-05 |
| 102 | PPP2CB | PP2CB | Phosphatase | NM_004156 | M-003599-00-05 |
| 103 | PPP2CA | PP2CA | Phosphatase | NM_002715 | M-003598-00-05 |
| 104 | PKC | PKCa | PKA/PKC signaling | NM_002737 | M-003523-00-05 |
| 105 | PRKACG | PKA C-g | PKA/PKC signaling | NM_002732 | M-004651-00-05 |
| 106 | PRKACB | PKA C-b | PKA/PKC signaling | NM_002731 | M-004650-00-05 |
| 107 | AKAP | AKAP1/PRKA1 | PKA/PKC signaling | NM_003488 | M-005181-00-05 |

The results of silencing these individual genes in living cells were assessed with a panel of 25 assays designed to report different hierarchical levels within known signaling pathways. The panel of 25 well-validated protein-protein interactions was selected to report on key nodes within the hierarchy of the diverse signaling pathways chosen for this study.

TABLE 9
Assay panel used for siRNA profiling

| | | | | Reporter 1 | | Reporter 2 |
|---|---|---|---|---|---|---|
| | | | Gene 1 | Fusion | Gene 2 | Fusion |
| Assay # | PCA Pair | Stimulus, conc (time) | Accession | Orientation | Accession | Orientation |
| 1 | Chk1/Cdc25C | CPT^{†}, 500 nM (18 hr) | NM_001274 | N | NM_001790 | C |
| 2 | Chk1/Cdc25C | | NM_001274 | N | NM_001790 | C |
| 3 | Mdm2/p53 | | NM_002392 | N | NM_000546 | C |
| 4 | p53/p53 | CPT^{†}, 500 nM (18 hr) | NM_000546 | C | NM_000546 | C |
| 5 | p53/p53 | | NM_000546 | C | NM_000546 | C |
| 6 | Mek1/Erk2 | | Z16415 | C | NM_002745 | C |
| 7 | Erk2/Elk1 | pDCR.RasV12^{‡}, 1 ng | NM_002745 | C | NM_005229 | C |
| 8 | Erk2/Elk1 | pDCR, 1 ng | NM_002745 | C | NM_005229 | C |
| 9 | Cdc2/Cdc25A | CPT^{†}, 500 nM (18 hr) | NM_001789 | N | NM_001786 | C |
| 10 | Cdc2/Cdc25A | | NM_001789 | N | NM_001786 | C |
| 11 | H-Ras/Raf-1 | | NM_005343 | N | NM_002880 | C |
| 12 | Pin1/Jun | pDCR, 1 ng | NM_006221 | C | NM_002228 | C |
| 13 | Raf-1/Mek1 | | NM_002880 | C | Z16415 | C |
| 14 | Chk1/p53 | | NM_001274 | N | NM_000546 | C |
| 15 | Akt1/p70S6K | | NM_005163 | C | NM_003161 | N |
| 16 | CBP/Rela (p65) | TNFα, 50 ng/ml (30 min) | AY079443 | C | NM_009045 | C |
| | | | (nt 1 . . . 2313) | | | |
| 17 | Cofilin/Limk2 | pcDNA3, 10 ng | NM_005507 | C | BC_013051 | N |
| 18 | Cofilin/Limk2 | pcDNA3.RacV12^{#}, 10 ng | NM_005507 | C | BC_013051 | N |
| 19 | Pin1/Jun | pDCR.RasV12^{‡}, 1 ng | NM_006221 | C | NM_002228 | C |
| 20 | Stat1/Stat1 | | NM_007315 | C | NM_007315 | N |
| 21 | CyclinD/Cdk4 | | NM_053056 | N | NM_001791 | C |
| 22 | Akt1/Hsp90β | | NM_005163 | C | NM_007355 | C |
| 23 | PPARγ/SRC-1 | Rosi^{¥}, 15 μM (1.5 hr) | NM_138712 | C | U40396 | N |
| | | | | | (nt 624 . . . 1256) | |
| 24 | eIF4E/eIF4G | | NM_001968 | C | NM_198244 | C |
| 25 | ITGβ1/ITGα5 | | NM_002211 | C | NM_002205 | C |

For each assay, HEK293 cells were transiently co-transfected with a pair of PCA vectors and siRNA, then stimulated with agonists as appropriate. The assays were categorized according to the subcellular localization of the fluorescent signal, and changes in signal intensity across each sample population (12 images per sample; ˜2,400 cells per sample) were quantified using one of three automated image analysis algorithms (see Methods). The effect of each siRNA pool on the fluorescence intensity of each assay was compared to the pooled mean fluorescence of two control (non-specific) siRNAs. Twenty-six of these siRNA pools directly targeted one of the components of a PCA, serving as a control for siRNA efficacy. The remaining 81 siRNA pools, however, targeted only endogenous proteins, allowing analyses of the effects of endogenous protein knockdown on pathway activity.

siRNA Clustering and Pathway Analysis

Combining the quantitative results from all 107 siRNA pools and the panel of 25 cell-based assays generated unique “fingerprints” or activity profiles, and unsupervised hierarchical clustering identified relationships between these profiles. A matrix of assay results and dendogram of unsupervised hierarchical clustering of siRNAs based on their activity on all 25 assays is shown in FIG. 20. Each column in the matrix corresponds to an individual siRNA pool (as shown at the bottom of the matrix), each row is a single assay (PCA/stimulus), listed on the left side of the figure. Each data point within the matrix is color coded to illustrate relative differences within an assay. For each row, the dynamic range of the values (reported as log ratio of sample/control) is separated into 9 levels. An increase relative to the control value is displayed as green and a decrease is displayed as red. Each color is further divided into four levels: level 1 (>75%), level 2 (>=50% and <75%), level 3 (>=25% and <50%), level 4 (>0 and <25%). Level one is displayed as the brightest hue and level 2 as the darker. Levels 3 and 4 are shaded in black. The dendogram at the top of the matrix was created with the Ward clustering algorithm utilizing Euclidean distance metrics. The height on the y-axis (distance between clusters) is not drawn to scale.

Examination of the clusters reveals both expected (on-pathway) and unexpected (off-pathway or novel) effects of siRNAs. For example, we observed a cluster of siRNAs targeting TNF-alpha and NF-kappa-B pathway components including TNFR, RIP2, TRADD, and NFKB1B (1-kappa-B-alpha). FIG. 20 also shows quantitative profiles of four siRNAs (Bcl-xL, TRADD, TNFR1, and NFKB1B. Each bar represents the fluorescence intensity for a given assay normalized to the appropriate control (percent of control). Measurements that differed significantly from the control represent the mean of triplicate measurements from three independent experiments. Silencing of the TNFR and TNF-receptor-proximal pathway members (TNFR1, TRADD, and Rip2) resulted in increases of both MAPK interactions and DNA damage-response interactions (FIG. 20). Notably, the PPAR-gamma:SRC-1 complex was also increased by siRNAs in this cluster, consistent with previous reports of negative regulation of PPAR-gamma activity by TNF-alpha through the NF-kappa-B pathway. TNF receptors are known to initiate both apoptotic and anti-apoptotic responses. Interestingly, siRNAs targeting IKBKB (IKK-alpha), and the anti-apoptotic Bcl2 and Bcl-xL also had similar effects on these assays, and thus clustered with this group.

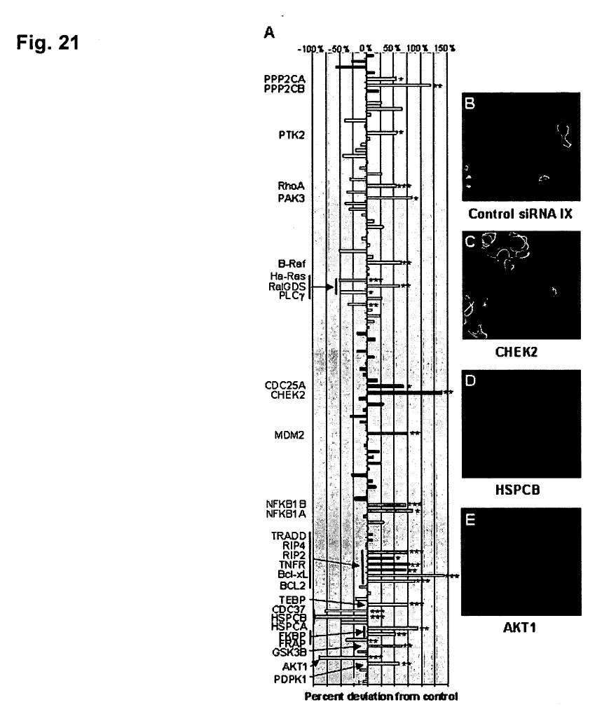

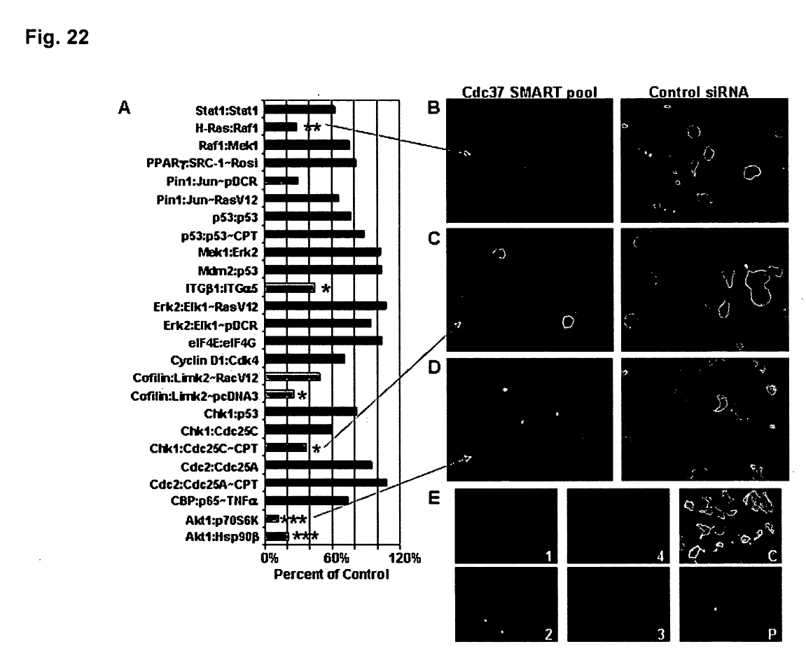

Clustering by Euclidean distance metrics indicates similarities in siRNA effects within and across pathways, an approach whose power increases as the number of assays and targets (e.g. siRNAs) increases. However, evaluating the effects on a pathway of silencing a single protein, or the ability to identify unexpected outcomes of target knockdown require more detailed, quantitative analysis. For instance, an unexpected link between a siRNA target and a specific pathway may be revealed by a single significant change in only one assay. Detailed quantitiatve analysis provides a means to identify potential therapeutic targets and also reveals whether inhibition of a specific protein has predictable or more pleiotropic effects on pathways. These details can be captured by examining a broad spectrum of siRNAs against a single signaling node (FIG. 21), and conversely, by examining the effect of a single siRNA against a broad spectrum of cellular assays (FIG. 22).

Assessing the Activity of Specific Signaling Nodes

An example of silencing a single signal transduction node is shown with the Akt1:Hsp90-beta assay (FIG. 21). Akt1 (PKB) plays a central role in the regulation of glucose transport and metabolism, cell growth, protein synthesis and apoptotic signals. Hsp90 is a molecular chaperone that plays an essential role in many biological processes by associating with a wide variety of proteins, including many protein kinases. Complexes of Akt with Hsp90 and the co-chaperone Cdc37 are thought to maintain Akt in a catalytically active state by preventing PP2A-dependent dephosphorylation and subsequent proteasome-mediated degradation. FIG. 21 shows the effects of 107 targeted siRNA pools on the Akt1:Hsp90-beta assay. In FIG. 21 (A), the fluorescence intensity (BulkSum) for each siRNA treatment from two-three independent transfections was normalized to the pooled mean from two non-specific siRNAs. Data are expressed as the percent deviation from the control. Inhibition relative to the control is displayed to the left of the y-axis, while stimulation is displayed to the right. Statistically significant measurements (ANOVA) are indicated with asterisks as follows: *, p≦0.05; **, p≦0.005; ***, p≦0.0005. The siRNAs associated with highly significant effects (p≦0.005) are indicated on the left side of the figure. The siRNAs were grouped by common pathway or function (FIG. 21B-E). Representative images of the effects of four siRNA SMART pools on the Akt1:Hsp90-beta assay are shown: (B) control siRNA IX, (C) siCHEK2, (D) siHSPCB (Hsp90-beta) and (E) siAKT1. All images were acquired with a 40× objective for the same exposure time. Hoechst (blue) and YFP (green) images were overlaid using Metamorph software (Molecular Devices).

siRNA-mediated knockdown of several functionally diverse targets also unexpectedly increased the number of Akt:Hsp90-beta complexes (FIG. 21). For example, siRNA-mediated knockdown of the DNA damage checkpoint proteins Chk2 and Cdc25A significantly increased the number of Akt:Hsp90-beta complexes (FIG. 21 A, C). siRNA-mediated silencing of several Akt substrates, including GSK3-beta, FRAP and Mdm2, all increased the number of Akt:Hsp90 complexes, implicating these proteins in negative feedback regulation of Akt. Consistent with the role of PP2A in promoting turnover of Akt, knockdown of either PP2A subunit also increased the Akt:Hsp90-beta complexes (p<0.05 and p<0.005, respectively). Similarly, silencing of RhoA, which negatively regulates PI3K/Akt signaling in endothelial cells, resulted in a significant increase in Akt:Hsp90-beta complexes (p<0.0001; FIG. 1A). Thus, by probing cellular signaling pathways in this fashion, we observed both expected (on-pathway) and unexpected (off-pathway) effects of the targets.

Defining Target Profiles

We also studied the effects of individual siRNAs against the entire panel of 25 assays. A number of kinases in the assay set were known to interact physically or genetically with the molecular chaperones Cdc37 and/or Hsp90, including Akt, Raf, Cdk4, and Chk1, prompting evaluation of the effect of siRNA targeting Cdc37 on the entire panel. FIG. 22 shows the effects of silencing Cdc37 on 25 assays. As shown in FIG. 22A-D, co-transfection of siCdc37 significantly decreased a number of protein complexes, including H-Ras:Raf-1, Akt1:p70S6K, Akt1:Hsp90-beta and Chk1:Cdc25C (p≦0.0001). Results for assays inhibited by ≧50% are depicted in magenta. Statistically significant results are indicated with asterisks (*, p≦0.05; **, p≦0.005 and ***, p≦0.0005). FIG. 22 B-D shows representative images for the effects of siCdc37 on three assays relative to a control siRNA: (B) H-Ras:Raf, (C) Chk1:Cdc25C (+CPT), and (D) Akt1:p70S6K. Images were acquired with a 20× objective on the Discovery-1 automated image analysis platform. FIG. 22E shows representative images for the effects of the Cdc37 SMART pool components on the Akt1:p70S6K assay, where (C) represents treatment with the siRNA control, (P) shows the effect of the SMART pool, and (1-4) indicate the four component Cdc37 siRNAs. Interestingly, treatment with pharmacological inhibitors of HSPs (geldanamycin and 17-allylamino geldanamycin) also resulted in similar decreases in the protein-protein complexes (Example 2 of this invention). Since Cdc37 acts as the direct interface between Hsp90 and its kinase substrates, our data support the central role of Cdc37 in stabilizing/activating these protein complexes.

To confirm the utility of the siRNA pooling strategy, all four components of the Cdc37 SMART pool were tested against all assays inhibited by the original SMART pool. As shown in FIG. 22E, all four siRNAs contributed similarly to the effect on the Akt1:p70S6K assay and similar results were obtained for the other seven assays highlighted in FIG. 22A (data not shown).

Optimizing Pathway Targeting with PCA and siRNA

Our siRNA collection was selected to target proteins whose activities impinge upon a number of pathways and at different levels of hierarchy. It would be interesting to compare the results of silencing a general modulator or integrator of diverse signaling cascades versus a direct effector of one of the affected pathways, as exemplified by H-Ras and its direct downstream effector, Raf-1. Ras-family small GTPases are central regulators of diverse cellular processes, including cell proliferation, cell motility and oncogenic transformation. The transforming potential of Ras is mediated in part through activation of the PI3K and Raf/MAPK cascades. Ras also stimulates the JNK/stress-activated pathway, which ultimately results in activation of the transcriptional potential of nuclear proteins such as c-Jun. Our assay panel included proteins representing key interactions in the PI3K and Raf/MAPK and JNK cascades. We therefore evaluated how silencing of H-Ras affected these assays.

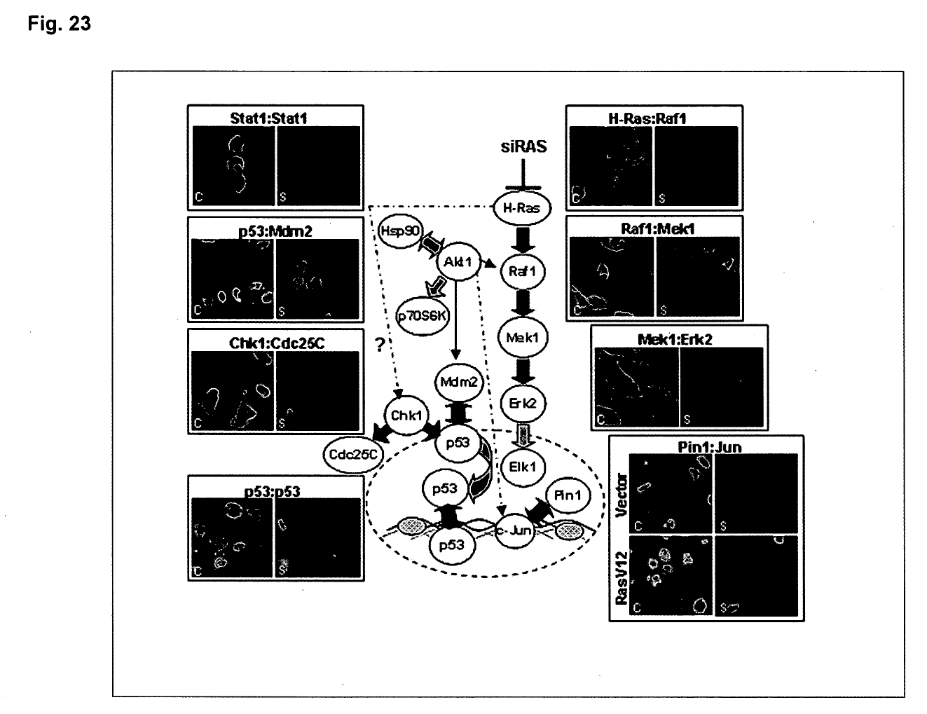

As shown in FIG. 23, treatment of cells with H-Ras siRNA resulted in ≧50% decrease (p≦0.001) of the H-Ras:Raf-1, Raf-1:Mek and Mek1:Erk2 complexes within the Raf/Mek cascade. Furthermore, interaction of c-Jun with the prolyl isomerase Pin1 (an indicator of phosphorylation of c-Jun) was also significantly reduced (>80%, p≦0.0001), both in the presence and absence of stimulation with a constitutively active H-Ras mutant (H-Ras(V12)). Additionally, components of the damage response pathway, specifically Chk1:Cdc25C, Chk1:p53, Mdm2:p53 and p53:p53 were reduced by at least 65% (p≦0.0001), and the cell cycle complex CyclinD:Cdk4 was reduced by 75% (p≦0.0001).

Despite the close physical and functional association of the proto-oncogene proteins H-Ras and Raf-1, the activity profiles generated by their cognate siRNAs were surprisingly distinct. In contrast to the broad activity of the H-Ras siRNA, the siRNA pool targeting the Ras effector Raf-1 significantly reduced (p≦0.0001) only two complexes: H-Ras:Raf-1 and Raf-1:Mek1 (Figure S1 and data not shown). The Raf-1 siRNA activity profile was more typical of the majority of siRNAs in the panel, which inhibited their target proteins and radiated their effects to one or two additional signaling nodes. Both Ras and Raf have been the subject of extensive drug discovery efforts, and inhibitors of both proteins are currently undergoing clinical analysis as oncology therapeutics. The significant difference between the profiles for these two targets reinforces an important point: not all proteins in a pathway are of equivalent value as therapeutic targets, and a generally applicable strategy for differentiating targets within a pathway is desirable.

Illuminating Novel Pathways and Targets

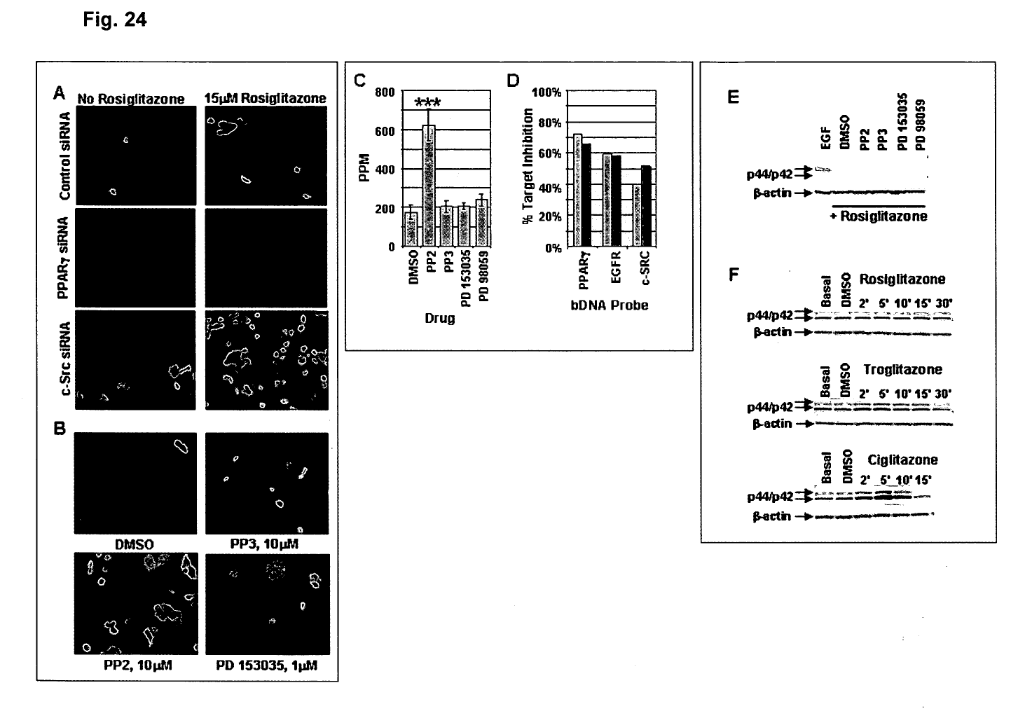

A primary goal of RNAi studies is the discovery of novel consequences of target inhibition that may be therapeutically relevant. We observed several examples of unexpected cellular activities of siRNAs targeting previously well-characterized signaling entities. A striking example of this was the induction of PPAR-gamma signaling complexes by silencing of the non-receptor tyrosine kinase c-src. FIG. 24(A) shows that co-transfection of c-src siRNA increases the PPAR-gamma:SRC-1 signal, both in the presence (right) and absence (left) of stimulation with 15 micromolar rosiglitazone for 90 minutes. In the presence of rosiglitazone, siRNA-mediated knockdown of c-src resulted in a more than 8-fold increase in nuclear receptor PPAR-gamma complex with the transcriptional co-activator SRC-1 (PPAR-gamma:SRC-1) compared to control siRNA (FIG. 24A). We confirmed a biochemical role for c-Src in the process with the c-Src selective kinase inhibitor PP2 which produced an effect comparable to c-src siRNA, increasing the PPAR-gamma:SRC-1 complex 5-fold (p<0.0001). An analog of PP2 which is inactive on c-Src (PP3) did not mimic the effects of either the c-src siRNA or of the active PP2 molecule. These results are illustrated in FIG. 24 (B) wherein HEK293 cells transiently transfected with the PPAR-gamma:SRC-1 PCA were serum-starved for 16 hours then treated with 10 micromolar PP2, 10 micromolar PP3, 1 micromolar PD 1153035, 10 micromolar PD 98059 or vehicle for 6.5 hours prior to stimulation with rosiglitazone for 1.5 hours. Representative images of drug effects are shown. In FIG. 24(C), data plotted for each drug treatment represent the mean (PPM) and standard error from 4 replicate wells in a minimum of two independent experiments. The effect of PP2 was highly significant (p<0.0001) relative to the DMSO control.

A selective inhibitor of the EGF receptor (PD 153035) as well as the MEK1 inhibitor PD 98059 had no appreciable effects on PPAR-gamma (FIG. 24B, C). As shown in FIG. 24D, Significant reductions in endogenous mRNA levels were observed for the PPAR-gamma, EGFR and c-src siRNA pools relative to pooled controls confirming the activity of these reagents. For FIG. 24 (D) HEK293 cells were transfected with the indicated siRNAs (40 nM) or two control siRNAs in the presence (maroon) or absence (blue) of the PPAR-gamma:SRC-1 PCA. Quantitation of inhibition of each target mRNA (PPAR-gamma, EGFR, and c-Src) was performed with bDNA probes (GenoSpectra) designed for each target gene. Percent inhibition was normalized to the effects of the pooled negative control siRNAs.

Previous studies suggested a central role for the EGFR and MAPK cascade in repressing PPAR-gamma activity in dividing cells. However, EGFR- and MAPK-targeted siRNAs in our panel had no significant effect on the assay. FIG. 24 (E) shows a western blot of phosphorylation status of p44/42 MAPK/ERK in HEK293 cells stimulated with EGF (Lane 1) or rosiglitazone (Lanes 2-6), in combination with PP2, PP3, PD 153035 or PD 98059. HEK293 cells were serum-starved overnight then pre-treated with DMSO, 10 micromolar PP2 or PP3, 1 micromolar PD 153035 or 20 micromolar PD 98059 for 1 hour prior to stimulation with rosiglitazone for 5 minutes. Cells stimulated with EGF (100 ng/ml for 5 minutes) served as a positive control. In FIG. 24 (F) Hep3B cells were serum starved overnight, then treated with PPAR-gamma agonists rosiglitazone, troglitazone and ciglitazone (50 micromolar each) for the indicated times. The phosphorylation status of p44/42 MAPK/ERK was compared to that of unstimulated (basal) or vehicle-treated (DMSO) cell extracts. All blots were normalized by re-probing with antibody to alpha-actin. Among the compounds we tested, only ciglitazone had a significant effect on ERK phosphorylation in Hep3B cells (FIG. 24F). Further, PPAR-gamma agonists did not elicit c-Src activation in 293 or Hep3B cells (data not shown).

Our results suggest that c-Src plays a significant and more direct role in modulating the activity of PPAR-gamma than previously suspected. Specifically, our data suggest that c-Src negatively regulates PPAR-gamma transcriptional complexes, and modulation of this effect does not occur via the EGFR/MAP kinase pathways, nor does it involve c-Src or EGFR/ERK activation by nuclear receptor agonists. These data are the first to directly demonstrate c-Src-mediated regulation of PPAR-gamma and to describe a general strategy for direct analysis of nuclear receptor signaling in living cells. Currently there is intense interest in targeting PPAR-gamma activity as a strategy for treating metabolic and proliferative disorders. Therefore, the identification of a link between c-Src and PPAR-gamma may provide additional drug-able targets for therapeutic intervention.

In the example presented in Example 3 we used live cell, pathway-based analyses to generate profiles of siRNA activity as a function of gene silencing. Each siRNA pool generated a unique profile of activity across the assay set confirming the utility of this network biology approach. These profiles illuminate similarities between targets involved in signal transduction. Many of these associations would be expected, and validate the predictive power of this approach. siRNAs that target the expression of proteins with distinct biochemical roles, but involved in the same pathway or biological process (such as TNFR1 and TRADD) regulate signaling pathways in similar ways based on their ‘on-pathway’ activities. Finally, an important feature of the approach is the ability to identify unexpected connections in previously well-characterized signaling pathways, as shown with our example of c-Src modulation of PPAR-gamma activity. Comparisons of RNAi- and drug-mediated effects on cellular networks are particularly valuable for defining drug and drug target mechanism of action. Numerous drugs routinely used as human therapeutics act, at least in part, by unknown mechanisms or have hidden phenotypes. By comparing the profiles of these drugs with large panels of siRNAs, an understanding of the proteins and pathways contributing to drug activity can be determined. Conversely, profiles of drugs with a particular therapeutic activity can be compared with RNAi profiles, leading to identification of novel therapeutic targets.

Detailed Methods for Example 3

107 siRNA SMART pools designed to target human genes (Table 8) and two ‘GC-match’ non-specific siRNAs (Dharmacon, Boulder, Colo.) were resuspended per the manufacturer's recommendations. PCA fusion-reporter constructs were produced as described for Example 2 above. Transfections were performed in HEK293 cells with 100 ng of nucleic acid per well (up to 50 ng of each fusion construct, and the appropriate siRNA SMART pool at 40 nM final concentration) with Lipofectamine 2000 (Invitrogen). For each screen, transfections were aliquoted in triplicate such that each assay, containing a single PCA pair, spanned four 96-well plates. Each 96-well plate contained five internal controls: mock (no PCA), no siRNA, non-specific siRNA controls IX and XI (47% and 36% GC content, respectively), and a PCA-specific control (to confirm degree of stimulation for assays treated with agonists). cDNAs were of human origin unless otherwise noted in Table 1. Optimal siRNA concentration was determined by evaluating the effects of siGFP (Dharmacon) and the non-specific siRNA controls on four different PCAs (data not shown). Images were acquired and analyzed as for Example 2.

Scope of the Present Invention

It will be understood by one skilled in the art that the present invention is not limited to the exact pathway, assay sentinel, assay protocol, detection method, or to particular instrumentation or software. The present invention teaches that cell-based fluorescence or luminescence assay panels can be used for pharmacological profiling of drugs, biologic agents, natural products, and other compounds of interest.

There is virtually no limit on the types, numbers, or types of the states and transitions that can be used in conjunction with this invention. There are likely to be thousands of such parameters that could provide information relevant for pharmacological profiling. These will be either constitutive or dynamic; and either redundant or non-redundant. Dynamic (responsive), non-redundant assays will be the most useful for pharmacological profiling as they will respond to pathway perturbations. Fortunately, one can determine empirically whether a specific state or transition is useful in profiling, by simply constructing an assay for the modification and testing it for responsiveness against a range of drugs, gene annotation reagents—such as siRNA—or other compounds. A non-redundant assay is one that provides distinct information, beyond the information provided by any other assay. As the pathways regulating cellular function are gradually elucidated it will eventually be possible to construct a completely predictive assay panel based on the methods provided herein. It will be possible to determine whether the panel is predictive by comparing the profiles of well-characterized agents that cause particular adverse effects in animals or in man, with the profiles of agents that do not cause the same effects. Such a panel would enable testing of any compound to determine its spectrum of activities and to determine any off-pathway activities suggestive of adverse consequences. The advantage of the approach is that it can be performed in high throughput such that thousands of lead compounds can be tested, prior to clinical studies, allowing early attrition of compounds with undesirable profiles.

Being genetically encoded, measurements of states and transitions can potentially be made in transgenic animals or in tissue xenografts, offering the possibility to perform imaging of signal transduction in live, whole organisms. Multiphoton excitation microscopy allows imaging in thick tissues, and a 2-photon, miniaturized microscope for imaging the brain of freely moving rats has been reported. A luficerase PCA has been used for this purpose in mice. Therefore, pharmacological profiling according to the present invention can be performed in whole animals and other model organisms.

The following patents including all those mentioned and cited in their specification, published patent applications as well as all their foreign counterparts and all cited references therein are incorporated in their entirety by reference herein as if those references were denoted in the text:

| | |
|---|---|
| 6,270,964 | Michnick, et al. |
| 6,294,330 | Michnick, et al. |
| 6,428,951 | Michnick, et al. |
| 5,989,835 | Dunlay, et al. |
| 6,518,021 | Thastrup, et al. |
| 5,583,217 | Quante, et al. |
| 5,516,902 | Quante, et al. |
| 5,514,561 | Quante, et al. |
| 5,338,843 | Quante, et al. |

### PUBLICATIONS

Pelletier, J. N., Remy, I. and Michnick, S. W. (1998). Protein-Fragment Complementation Assays: a General Strategy for the in vivo Detection of Protein-Protein Interactions. Journal of Biomolecular Techniques, 10: 32-39.
Remy, I. and Michnick, S. W. (1999). Clonal Selection and In Vivo Quantitation of Protein Interactions with Protein Fragment Complementation Assays. Proc Natl Acad Sci USA, 96: 5394-5399.
Remy, I., Pelletier, J. N., Galarneau, A. and Michnick, S. W. (2002). Protein Interactions and Library Screening with Protein Fragment Complementation Strategies. Protein-protein interactions: A molecular cloning manual. E. A. Golemis, editor. Cold Spring Harbor Laboratory Press. Chapter 25, 449-475.
Remy, I., Wilson, I. A. and Michnick, S. W. (1999). Erythropoietin receptor activation by a ligand-induced conformation change. Science, 283: 990-993.
Galarneau, A., Primeau, M., Trudeau, L.-E. and Michnick, S. W. (2002). A Protein fragment Complementation Assay based on TEM1 β-lactamase for detection of protein-protein interactions. Nat Biotechnol, 20: 619-622.
Michnick, S. W., Remy, I., C.-Valois, F. X., Vallee-Belisle, A., Galarneau, A. and Pelletier, J. N. (2000) Detection of Protein-Protein Interactions by Protein Fragment Complementation Strategies, Parts A and B (John N. Abelson, Scott D Emr and Jeremy Thorner, editors) A Volume of Methods in Enzymology. 328, 208-230.
Remy, I. and Michnick, S. W. (2001). Visualization of Biochemical Networks in Living Cells. Proc Natl Acad Sci USA, 98: 7678-7683.
Xia Y., et al. ANALYZING CELLULAR BIOCHEMISTRY IN TERMS OF MOLECULAR NETWORKS, in: Annual Review of Biochemistry Vol. 73: 1051-1087 (2004)
Current Protocols in Immunology, ed J. E. Coligan et al., J. Wiley & sons, New York 1991, ISBN 0-471-52276-7.
Brand, L. and Johnson, M. L., Eds., Fluorescence Spectroscopy (Methods in Enzymology, Volume 278), Academic Press (1997).
Cantor, C. R. and Schimmel, P. R., Biophysical Chemistry Part 2, W.H. Freeman (1980) pp. 433-465.
Dewey, T. G., Ed., Biophysical and Biochemical Aspects of Fluorescence Spectroscopy, Plenum Publishing (1991).
Guilbault, G. G., Ed., Practical Fluorescence, Second Edition, Marcel Dekker (1990).
Lakowicz, J. R., Ed., Topics in Fluorescence Spectroscopy: Techniques (Volume 1, 1991); Principles (Volume 2, 1991); Biochemical Applications (Volume 3, 1992); Probe Design and Chemical Sensing (Volume 4, 1994); Nonlinear and Two-Photon Induced Fluorescence (Volume 5, 1997); Protein Fluorescence (Volume 6, 2000); DNA Technology (Volume 7, 2003); Plenum Publishing.
Lakowicz, J. R., Principles of Fluorescence Spectroscopy, Second Edition, Plenum Publishing (1999).
Mathies, R. A., Peck, K. and Stryer, L., “Optimization of high-sensitivity fluorescence detection,” Anal Chem 62, 1786-1791 (1990).
Agbaria, R. A., Oldham, P. B., McCarroll, M. E., McGown, L. B. and Warner, I. M., “Molecular fluorescence, phosphorescence, and chemiluminescence spectrometry,” Anal Chem 74, 3952-3962 (2002).
Royer, C. A., “Approaches to teaching fluorescence spectroscopy,” Biophys J 68, 1191-1195 (1995).
Sharma, A. and Schulman, S. G., Introduction to Fluorescence Spectroscopy, John Wiley and Sons (1999).
Valeur, B., Molecular Fluorescence: Principles and Applications, John Wiley and Sons (2002).
Berlman, I. B., Handbook of Fluorescence Spectra of Aromatic Molecules, Second Edition, Academic Press (1971).
Czarnik, A. W., Ed., Fluorescent Chemosensors for Ion and Molecule Recognition (ACS Symposium Series 538), American Chemical Society (1993).
Drexhage, K. H., “Structure and properties of laser dyes” in Dye Lasers, Third Edition, F. P. Schäfer, Ed., Springer-Verlag, (1990) pp. 155-200.
Giuliano, K. A., Post, P. L., Hahn, K. M. and Taylor, D. L., “Fluorescent protein biosensors: measurement of molecular dynamics in living cells,” Annu Rev Biophys Biomol Struct 24, 405-434 (1995).
Green, F. J., The Sigma-Aldrich Handbook of Stains, Dyes and Indicators, Aldrich Chemical Company (1990).
Griffiths, J., Colour and Constitution of Organic Molecules, Academic Press (1976).
Haugland, R.P., “Antibody conjugates for cell biology” in Current Protocols in Cell Biology, J. S. Bonifacino, M. Dasso, J. Lippincott-Schwartz, J. B. Harford and K. M. Yamada, Eds., John Wiley and Sons (2000) pp. 16.5.1-16.5.22.
Haugland, R. P., “Spectra of fluorescent dyes used in flow cytometry,” Meth Cell Biol 42, 641-663 (1994).
Hermanson, G. T., Bioconjugate Techniques, Academic Press (1996).
Johnson, I. D., Ryan, D. and Haugland, R. P., “Comparing fluorescent organic dyes for biomolecular labeling” in Methods in Nonradioactive Detection, G. C. Howard, Ed., Appleton and Lange (1993) pp. 47-68.
Johnson, I. D., “Fluorescent probes for living cells,” Histochem J 30, 123-140 (1998).
Kasten, F. H., “Introduction to fluorescent probes: properties, history and applications” in Fluorescent and Luminescent Probes for Biological Activity, W. T. Mason, Ed., Academic Press (1993) pp. 12-33.
Krasovitskii, B. M. and Bolotin, B. M., Organic Luminescent Materials, VCH Publishers (1988).
Lakowicz, J. R., Ed., Topics in Fluorescence Spectroscopy: Probe Design and Chemical Sensing (Volume 4), Plenum Publishing (1994).
Mason, W. T., Ed., Fluorescent and Luminescent Probes for Biological Activity, Second Edition, Academic Press (1999). Available from Molecular Probes (F14944, Section 23.6).
Marriott, G., Ed., Caged Compounds (Methods in Enzymology, Volume 291), Academic Press (1998).
Tsien, R. Y., “The green fluorescent protein,” Annu Rev Biochem 67, 509-544 (1998).
Waggoner, A. S., “Fluorescent probes for cytometry” in Flow Cytometry and Sorting, Second Edition, M. R. Melamed, T. Lindmo and M. L. Mendelsohn, Eds., Wiley-Liss (1990) pp. 209-225.
Wells, S, and Johnson, I., “Fluorescent labels for confocal microscopy” in Three-Dimensional Confocal Microscopy: Volume Investigation of Biological Systems, J. K. Stevens, L. R. Mills and J. E. Trogadis, Eds., Academic Press (1994) pp. 101-129.
Allan, V., Ed., Protein Localization by Fluorescence Microscopy: A Practical Approach, Oxford University Press (1999).
Andreeff, M. and Pinkel, D., Eds., Introduction to Fluorescence In Situ Hybridization: Principles and Clinical Applications, John Wiley and Sons (1999).
Conn, P. M., Ed., Confocal Microscopy (Methods in Enzymology, Volume 307), Academic Press (1999).
Denk, W. and Svoboda, K., “Photon upmanship: why multiphoton imaging is more than a gimmick,” Neuron 18, 351-357 (1997).
Diaspro, A., Ed., Confocal and Two-Photon Microscopy: Foundations, Applications and Advances, John Wiley and Sons (2001).
Herman, B., Fluorescence Microscopy, Second Edition, BIOS Scientific Publishers (1998).
Inoué, S, and Spring, K. R., Video Microscopy, Second Edition, Plenum Publishing (1997).
Matsumoto, B., Ed., Cell Biological Applications of Confocal Microscopy, Second Edition (Methods in Cell Biology, Volume 70), Academic Press (2003).
Michalet, X., Kapanidis, A. N., Laurence, T., Pinaud, F., Doose, S., Pflughoefft, M. and Weiss S., “The power and prospects of fluorescence microscopies and spectroscopies,” Annu Rev Biophys Biomolec Struct 32, 161-182 (2003).
Murphy, D. B., Fundamentals of Light Microscopy and Electronic Imaging, John Wiley and Sons (2001).
Pawley, J. B., Ed., Handbook of Biological Confocal Microscopy, Second Edition, Plenum Publishing (1995).
Paddock, S., Ed., Confocal Microscopy (Methods in Molecular Biology, Volume 122), Humana Press (1998).
Periasamy, A., Ed., Methods in Cellular Imaging, Oxford University Press (2001).
Rizzuto, R., and Fasolato, C., Eds., Imaging Living Cells, Springer-Verlag (1999).
Sheppard, C. J. R. and Shotton, D. M., Confocal Laser Scanning Microscopy, BIOS Scientific Publishers (1997).
Stevens, J. K., Mills, L. R. and Trogadis, J. E., Eds., Three-Dimensional Confocal Microscopy: Volume Investigation of Biological Systems, Academic Press (1994).
Taylor, D. L. and Wang, Y. L., Eds., Fluorescence Microscopy of Living Cells in Culture, Parts A and B (Methods in Cell Biology, Volumes 29 and 30), Academic Press (1989).
Toomre, D. and Manstein, D. J., “Lighting up the cell surface with evanescent wave microscopy,” Trends Cell Biol 11, 298-303 (2001).
Tsien, R. Y., “Imagining imaging's future,” Nat Rev Mol Cell Biol 4, SS16-SS21 (2003).
Wang, X. F. and Herman, B., Eds., Fluorescence Imaging Spectroscopy and Microscopy, John Wiley and Sons (1996).
Yuste, R., Lanni, F. and Konnerth, A., Imaging Neurons: A Laboratory Manual, Cold Spring Harbor Laboratory Press (2000).
Darzynkiewicz, Z., Crissman, H. A. and Robinson, J. P., Eds., Cytometry, Third Edition Parts A and B (Methods in Cell Biology, Volumes 63 and 64), Academic Press (2001).
Davey, H. M. and Kell, D. B., “Flow cytometry and cell sorting of heterogeneous microbial populations: the importance of single-cell analyses,” Microbiological Rev 60, 641-696 (1996).
Gilman-Sachs, A., “Flow cytometry,” Anal Chem 66, 700A-707A (1994).
Givan, A. L., Flow Cytometry: First Principles, Second Edition, John Wiley and Sons (2001).
Herzenberg, L. A., Parks, D., Sahaf, B., Perez, O., Roederer, M. and Herzenberg, L. A., “The history and future of the fluorescence activated cell sorter and flow cytometry: a view from Stanford,” Clin Chem 48, 1819-1827(2002).
Jaroszeski, M. J. and Heller, R., Eds., Flow Cytometry Protocols (Methods in Molecular Biology, Volume 91), Humana Press (1997).
Lloyd, D., Ed., Flow Cytometry in Microbiology, Springer-Verlag (1993).
Melamed, M. R., Lindmo, T. and Mendelsohn, M. L., Eds., Flow Cytometry and Sorting, Second Edition, Wiley-Liss (1990).
Ormerod, M. G., Ed., Flow Cytometry: A Practical Approach, Third Edition, Oxford University Press (2000).
Robinson, J. P., Ed., Current Protocols in Cytometry, John Wiley and Sons (1997).
Shapiro, H. M., “Optical measurement in cytometry: light scattering, extinction, absorption and fluorescence,” Meth Cell Biol 63, 107-129 (2001).
Shapiro, H. M., Practical Flow Cytometry, Fourth Edition, Wiley-Liss (2003).
Weaver, J. L., “Introduction to flow cytometry,” Methods 21, 199-201 (2000). This journal issue also contains 10 review articles on various flow cytometry applications.
Goldberg, M. C., Ed., Luminescence Applications in Biological, Chemical, Environmental and Hydrological Sciences (ACS Symposium Series 383), American Chemical Society (1989).
Gore, M., Ed., Spectrophotometry and Spectrofluorimetry: A Practical Approach, Second Edition, Oxford University Press (2000).
Hemmilä, I. A., Applications of Fluorescence in Immunoassays, John Wiley and Sons (1991).
Patton, W. F., “A thousand points of light: the application of fluorescence detection technologies to two-dimensional gel electrophoresis and proteomics,” Electrophoresis 21, 1123-1144 (2000).
Jackson, A. L. et al. Expression profiling reveals off-target gene regulation by RNAi. Nat Biotechnol 21, 635-7 (2003).
Hannon, G. J. RNA interference. Nature 418, 244-51 (2002).
Paddison, P. J. & Hannon, G. J. RNA interference: the new somatic cell genetics? Cancer Cell 2, 17-23 (2002).
Remy, I. & Michnick, S. W. Mapping biochemical networks with protein-fragment complementation assays. Methods Mol Biol 261, 411-26 (2004).
Nagai, T. et al. A variant of yellow fluorescent protein with fast and efficient maturation for cell biological applications. Nat Biotechnol 20, 87-90 (2002).
Yu, H. et al. Measuring drug action in the cellular context using protein-fragment complementation assays. Assay Drug Dev Technol 1, 811-22 (2003).

While the many forms of the invention herein disclosed constitute presently preferred embodiments, many others are possible and further details of the preferred embodiments and other possible embodiments are not to be construed as limitations. It is understood that the terms used herein are merely descriptive rather than limiting and that various changes and many equivalents may be made without departing from the spirit or scope of the claimed invention.

What is claimed is:

1. A composition comprising a panel of assays, wherein each assay of said panel is performed in a cell or cells, and wherein each assay comprises a measurement of one or more molecular parameters.

2. A composition according to claim 1 wherein said molecular parameters are selected from the group comprising (a) states of molecules; and (b) transitions of molecules.

3. A composition according to claim 2 wherein any of said transitions of molecules are selected from the group comprising: (a) chemical modification; (b) replication; (c) synthesis; (d) degradation; (e) transcription; (f) translation; (g) alternative splicing; (h) transportation; (i) non-covalent modification; (j) cleavage; (k) addition or removal; (l) allosteric change; (m) structural change; (n) redox change; (O) solubility change; (p) association; (q) dissociation; (r) interaction; (s) binding; and (t) multimerization.

4. A composition according to claim 2 wherein any of said states of molecules are selected from the group comprising (a) macromolecules; (b) small molecules; (c) complexes; (d) products of any transitions of any of (a)-(c); (e) quantities of any of (a)-(d); and (f) subcellular compartments of any of (a)-(f).

5. A composition according to claim 3, with reference to item (k) of claim 3, wherein said addition and removal are selected from the group comprising (a) phosphorylation/dephosphorylation; (b) methylation/demethylation; (c) fatty acylation/deacylation; (d) ubiquitination or SUMOylation; (e) epitope addition or loss; (f) glycosylation/deglycosylation; (i) removal or addition of a heme; (j) nitrosylation/denitrosylation; (k) oxidation/reduction; (l) acetylation/deacetylation; (m) myristylation/de-myristylation; (i) prenylation/deprenylation; (j) removal or addition of an amino acid or nucleotide; and (k) binding or loss of another molecule.

6. A composition according to claim 4, wherein any of said transitions of item (d) of claim 4 are selected from the group comprising (a) chemical modification; (b) replication; (c) synthesis; (d) degradation; (e) transcription; (f) translation; (g) alternative splicing; (h) transportation; (i) non-covalent modification; (j) cleavage; (k) addition or removal; (l) allosteric change; (m) structural change; (n) redox change; (o) solubility change; (p) association; (q) dissociation; (r) interaction; (s) binding; and (s) multimerization.

7. A method of analyzing a test chemical compound to identify an activity profile of said compound in a cell or cells, said method comprising the steps of: (A) constructing a panel of assays, wherein each assay is performed in a cell or cells, wherein each assay comprises a measurement of one or more molecular parameters; (B) contacting each of said cell(s) in said panel with a test chemical compound; (c) measuring the effects of said test chemical compound in said assays in said panel; and (d) using the results of step (c) to identify an activity profile for said chemical compound in said cells.

8. A method of determining a profile of activity of a test compound in a cell or cells, said method comprising the steps of:
(a) constructing a panel of assays, said panel comprising at least a first cell-based assay and a second cell-based assay, wherein each of said first cell-based assay and said second cell-based assay comprises a measurement of one or more molecular parameters;
(b) Contacting the first of two identical populations of cells from said first cell-based assay with a test chemical compound;
(c) Contacting the second of two identical populations of cells from said first cell-based assay with a vehicle or with no reagent;
(d) Comparing the results of step (b) and step (c) to determine the activity of said test chemical compound relative to the absence of said test chemical compound in said first cell-based assay;
(e) Contacting the first of two populations of identical cells from said second cell-based assay with said test chemical compound;
(f) Contacting the second of two populations of identical cells from said second cell-based assay with said vehicle or with no reagent;
(g) Comparing the results of steps e and f to determine the activity of said test chemical compound relative to the absence of said test chemical compound in said second cell-based assay; and
(h) Combining the results of step (c) and step (g) to establish an activity profile for said test chemical compound in said assay panel.

9. A method according to either of claims 7 or 8 wherein said molecular parameters are selected from the group comprising: (a) states; and (b) transitions.

10. A method for assessing the potential safety of a chemical compound, said method comprising (A) using the method of claim 13 to establish an activity profile of a test chemical compound in an assay panel; (B) using the method of claim 13 to establish an activity profile of a reference compound in said assay panel, said reference compound having established safety characteristics; (C) comparing said activity profile of said test chemical compound to said activity profile of said reference compound; (D) if said activity profile of said test chemical compound is substantially similar to said activity profile of said reference compound, determining that said chemical compound has potential safety characteristics substantially similar to those of said reference compound.

11. A method for assessing the potential toxic or adverse effects of a chemical compound, said method comprising (A) using the method of claim 13 to establish an activity profile of a test chemical compound in an assay panel; (B) using the method of claim 13 to establish an activity profile of a reference compound in said assay panel, said reference compound having established toxic or adverse characteristics; (C) comparing said activity profile of said test chemical compound to said activity profile of said reference compound; (D) if said activity profile of said test chemical compound is substantially similar to said activity profile of said reference compound, determining that said chemical compound has potential toxic or adverse characteristics substantially similar to those of said reference compound.

12. A method according to claims 1, 7 or 8 wherein said molecular parameter is selected from a molecule listed in Tables 6 or 6A.
